(19) United States
(12) Patent Application Publication
(10) Pub. No.: US 2025/0000884 A1
(43) Pub. Date: Jan. 2, 2025

(54) OLIGOSACCHARIDE COMPOSITIONS AND METHODS OF USE THEREOF FOR TREATING INFLAMMATORY LUNG DISEASES

(71) Applicant: DSM Nutritional Products, LLC, Parsippany, NJ (US)

(72) Inventor: James Raymond BROWN, Berwyn, PA (US)

(21) Appl. No.: 18/698,176

(22) PCT Filed: Oct. 3, 2022
(86) PCT No.: PCT/US2022/045490

§ 371 Date: Apr. 3, 2024

Related U.S. Application Data
(60) Provisional application No. 63/275,933, filed on Nov. 4, 2021.
(60) Provisional application No. 63/252,140, filed on Oct. 4, 2021.

(51) Int. Cl. A61K 31/702; A61K 35/747; A61P 31/16

(52) U.S. Cl. CPC A61K 31/702; A61K 35/747; A61P 31/16

(57) ABSTRACT

Aspects of the disclosure relate to methods of using oligosaccharide compositions as microbiome metabolic therapies for treating inflammatory lung diseases and disorders, such as chronic obstructive pulmonary disease (COPD) and asthma.

### RELATED APPLICATIONS

This application claims the benefit under 35 U.S.C. § 119 (e) of U.S. Provisional Application No. 63/252,140, filed Oct. 4, 2021; and U.S. Provisional Application No. 63/275,933, filed Nov. 4, 2021; the entire contents of each of which are incorporated herein by reference.

### FIELD OF THE INVENTION

The present disclosure relates to oligosaccharide compositions and uses thereof for treating inflammatory lung diseases (e.g., chronic obstructive pulmonary disease (COPD) and asthma).

### BACKGROUND OF INVENTION

Inflammatory lung and respiratory diseases which include chronic obstructive pulmonary disease (COPD), asthma, bronchiectasis and cystic fibrosis represent a major global healthcare burden. COPD is a lung disease characterized by chronic obstruction of lung airflow that interferes with normal breathing and has not been previously shown to be fully reversible. Furthermore, COPD is the third leading cause of death in the world with more than 3.17 million deaths in 2015 (5% of worldwide deaths in 2015). The Global Burden of Disease Study reports a prevalence of 251 million cases of COPD globally in 2016. The COPD burden is projected to increase in coming decades because of continued exposure to COPD risk factors (e.g., smoking, airborne particulates) and an aging global population. Current treatment options for inflammatory lung and respiratory diseases such as COPD include antibiotics, bronchodilators and corticosteroids. However, there is a great need for more effective and safer treatment options.

### SUMMARY OF INVENTION

Aspects of this disclosure relate to the recognition that a patient's gut microbiome can be modulated to modify the body's immune response and combat, e.g., inflammation of the lung. In particular, in some embodiments, microbiome metabolic therapies (MMTs) are provided that utilize oligosaccharide compositions to treat inflammatory lung diseases or disorders (e.g., chronic obstructive pulmonary disease (COPD) or asthma).

In some embodiments, a patient's gut microbiome is modulated to produce short-chain fatty acids (e.g., acetate, butyrate, and/or propionate) that act to reduce inflammation in the lungs and gut. Accordingly, in some embodiments, oligosaccharide compositions are utilized to stimulate production of such short-chain fatty acids by the gut microbiome that are more consistent and reproducible across patient samples than commercial fibers (see, e.g., FIGS. 1A-1B and FIG. 2).

In some embodiments, oligosaccharide compositions and related methods provided herein are useful for treating inflammatory lung diseases or disorders (e.g., chronic obstructive pulmonary disease (COPD), and asthma). In some embodiments, oligosaccharide compositions and related methods are useful for treating subjects with moderate, severe, or very severe COPD. In certain embodiments, such treatments minimize the need for hospitalization or extended hospitalized care. In certain embodiments, such treatments reduce the length of hospitalization stays. In certain embodiments, such treatments reduce the frequency, duration and/or severity of episodes of acute exacerbations of COPD (AECOPD). In certain embodiments, such treatments reduce the frequency, duration and/or severity of asthma attacks (including asthma attacks that result in hospitalization). In certain embodiments, such treatments minimize or prevent progression of the illness (e.g., progression from mild or moderate symptoms to severe symptoms, e.g., such that subject requires further intervention, e.g., a ventilator or respirator). In some embodiments, such treatments involve the administration of the oligosaccharide composition in combination with a standard-of-care treatment such as bronchodilators, antibiotics and corticosteroids. In some embodiments, standard-of-care treatments are self-administered. In some embodiments, standard-of-care treatments are home care treatments (e.g., administered at home, e.g., self-administered at home). In some embodiments, self-administered and/or home care treatments include bed rest, hydration, over-the-counter and or prescription medication. In some embodiments, such treatments involve the administration of the oligosaccharide composition following a standard-of-care treatment. In some embodiments, a standard-of-care treatment includes bronchodilators, antibiotics and corticosteroids. In some embodiments, a standard-of-care treatment is triple therapy (e.g., administration of long-acting muscarinic antagonists (LAMAs), long-acting inhaled β-agonists (LABAs) and inhaled corticosteroids (ICSs)). In some embodiments, a standard-of-care treatment is dual bronchodilation (LAMAs and LABAs). In some embodiments, a subject has been using a standard-of care-treatment (e.g., the same treatment) for at least 6, 9, 12, 15, 18, 24, or more months.

Short-chain fatty acids, in some embodiments, modulate the immune and inflammatory responses of a subject, for example in response to an inflammatory episode (e.g., an AECOPD episode). The methods described herein are, in some embodiments, based on the realization that the oligosaccharide compositions of the disclosure can provide consistent, reliable and beneficial health effects, e.g., by modulating short-chain fatty acid metabolism in the gastointestinal tract.

In further aspects, the disclosure relates to a recognition that modulating a patient's gut to produce short-chain fatty acids is useful for reducing the risk of pathogenic infections in subjects having an inflammatory lung disease or disorder. Accordingly, in some embodiments, oligosaccharide compositions of the disclosure are provided for preventing or attenuating pathogenic infections in subjects having an inflammatory lung disease or disorder. Pathogenic infections include, e.g., viral pathogens (e.g., respiratory viruses). In other embodiments, pathogenic infections include bacterial pathogens (e.g., including drug-resistant bacterial pathogens). In some embodiments, commensal microbe populations are modulated by the oligosaccharide compositions, e.g., to enhance protection from pathogen infection and pathogen colonization.

In some embodiments, a method of treating a subject having an inflammatory lung disease or disorder is provided. For example, the method, in some embodiments, comprises administering to the gastrointestinal tract of a subject an effective amount of an oligosaccharide composition, wherein the oligosaccharide composition has an average degree of polymerization of 5-20 and comprises a plurality of oligosaccharides selected from Formula (I), Formula (II), and Formula (III):

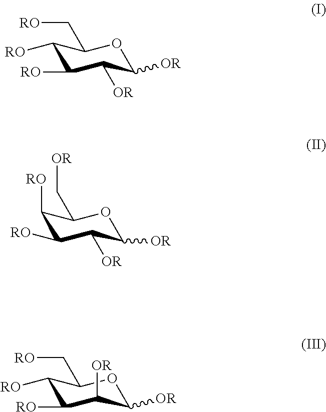

wherein each R independently is selected from hydrogen, and Formulae (Ia), (Ib), (Ic), (Id), (IIa), (IIb), (IIc), (IId), (IIIa), (IIIb), (IIIc), (IIId):

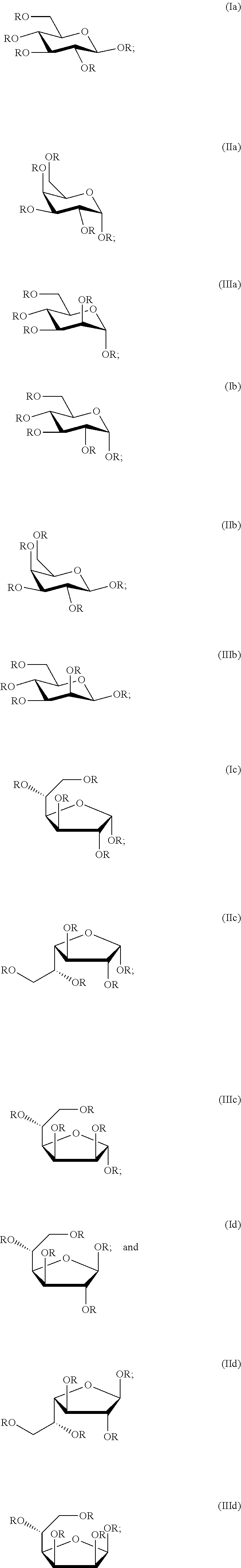

wherein each R independently is as defined above; thereby treating the subject.

In some embodiments, the disclosure is directed to a method of treating a subject having dysbiosis of the lung microbiome, the method comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I), Formula (II), and Formula (III):

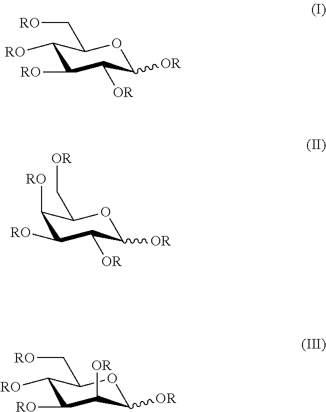

wherein each R independently is selected from hydrogen, and Formulae (Ia), (Ib), (Ic), (Id), (IIa), (IIb), (IIc), (IId), (IIIa), (IIIb), (IIIc), (IIId):

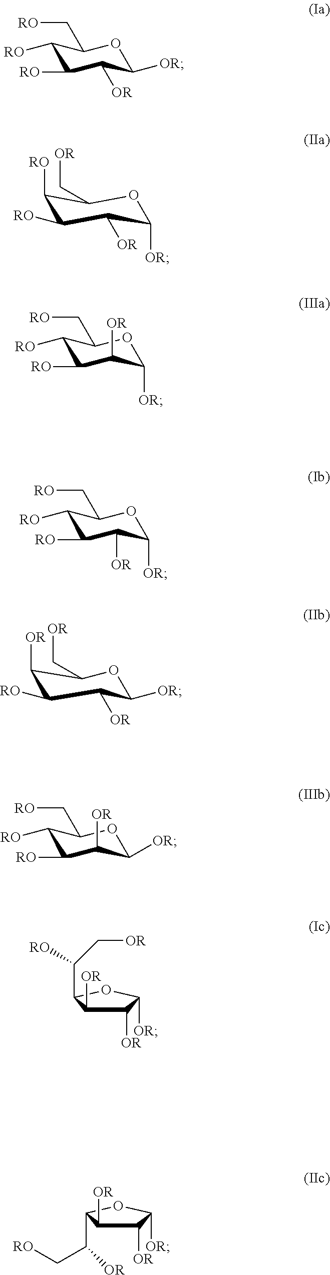

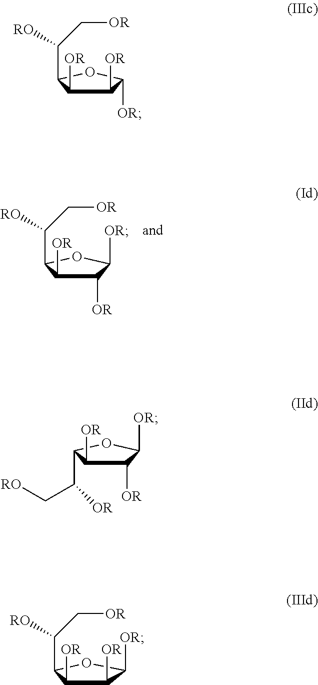

wherein each R independently is as defined above;
thereby attenuating an immune response in the subject.

Further methods of the disclosure comprise identifying a human subject having an inflammatory lung disease or disorder; and treating the subject with an oligosaccharide composition as described herein.

In some embodiments, the inflammatory lung disease or disorder is chronic. In some embodiments, the inflammatory lung disease or disorder is acute. In some embodiments, the inflammatory lung disease or disorder is chronic obstructive pulmonary disease (COPD), asthma, bronchiectasis, cystic fibrosis, pulmonary fibrosis, interstitial lung disease, tuberculosis, or allergy (e.g., an allergy causing respiratory problems, a skin allergy, a food allergy).

In some embodiments, the subject has dysbiosis of the lung microbiome. In some embodiments, the subject has dysbiosis of the gut microbiome.

In some embodiments, the inflammatory lung disease or disorder is chronic obstructive pulmonary disease (COPD). In some embodiments, the subject has moderate COPD (Global Initiative for Chronic Obstructive Lung Disease (GOLD) Stage II), severe COPD (GOLD Stage III), or very severe COPD (GOLD Stage IV). In some embodiments, the subject has a Forced Expiratory Volume in the first second of exhalation (FEV₁) of less than 80%. In some embodiments, the subject has had at least two acute exacerbations of COPD (AECOPD) events in the last 12 month. In some embodiments, the subject has been treated with triple therapy (long-acting muscarinic antagonists (LAMAs), long-acting inhaled β-agonists (LABAs) and inhaled corticosteroids (ICSs) or dual bronchodilation (LAMAs and LABAs) for the previous 12+ months. In some embodiments, the subject has moderate COPD (GOLD Stage II) or severe COPD (GOLD Stage III). In some embodiments, the subject has an FEV₁ of greater than 30% and less than 80% (e.g., FEV₁ of 30%-79%). In some embodiments, the subject has severe COPD (GOLD Stage III) or very severe COPD (GOLD Stage IV). In some embodiments, the subject has an FEV₁ of less than 49%. In some embodiments, the subject has very severe COPD (GOLD Stage IV). In some embodiments, the subject has an FEV₁ of equal to or less than 30%.

In some embodiments, the subject is more susceptible to episodes of acute exacerbations of COPD (AECOPD) relative to a healthy subject or a subject having GOLD Stage I COPD or GOLD Stage II COPD. In some embodiments, the subject experiences at least one, two, or more AECOPD episodes per year. In some embodiments, the AECOPD episodes have a duration of 5, 6, 7, 8, 9, 10, or more days. In some embodiments, the subject experiences at least one, two, or three AECOPD episodes per year that result in hospitalization of the subject. In some embodiments, the subject experiences fewer AECOPD episodes per year following administration of the oligosaccharide composition relative to a control subject (e.g., untreated subject) or a baseline measurement. In some embodiments, the subject experiences less severe and/or less frequent AECOPD episodes following administration of the oligosaccharide composition relative to AECOPD episodes of a control subject (e.g., untreated subject) or a baseline measurement. In some embodiments, following administration of the oligosaccharide composition, the subject experiences AECOPD episodes having a duration that is at least 10%, 20%, 30%, 40%, 50% or 75% shorter than their average AECOPD duration prior to the administration. In some embodiments, the subject experiences fewer AECOPD episodes that result in hospitalization of the subject per year (e.g., measured during a one-, two- or three-year period) following administration of the oligosaccharide composition relative to a control subject (e.g., untreated subject) or a baseline measurement.

In some embodiments, the subject has increased relative or absolute abundance of pathogenic bacteria in their lungs relative to a healthy subject. In some embodiments, administration of the oligosaccharide composition reduces the relative or absolute abundance of pathogenic bacteria in the lungs, sputum and/or gut of the subject relative to a control subject (e.g., untreated subject) or a baseline measurement. In some embodiments, the subject has increased levels of Moraxella catarrhalis, Haemophilus influenzae and/or Streptococcus pneumoniae in their lungs relative to a healthy subject. In some embodiments, administration of the oligosaccharide composition reduces the relative or absolute abundance of Moraxella catarrhalis, Haemophilus influenzae and/or Streptococcus pneumoniae in the lungs of the subject relative to a control subject (e.g., untreated subject) or a baseline measurement.

In some embodiments, the oligosaccharide composition promotes increased relative or absolute abundance of commensal bacteria in the lungs, sputum and/or gut of the subject. In some embodiments, the oligosaccharide composition promotes increased relative or absolute abundance of one or more of Lactobacillus, Parabacteroides and Bacteroides.

In some embodiments, the subject is more susceptible to respiratory viral infections than a healthy subject. In some embodiments, the respiratory viral infections are caused by one or more of influenza (FLU), human rhinovirus (HRV), respiratory syncytial virus (RSV) and coronaviruses (Family Coronaviridae).

In some embodiments, administration of the oligosaccharide composition reduces the number of inflammatory episodes (e.g., AECOPD episodes or asthma attacks), e.g., associated with bacterial infections. For example, bacterial infections associated with vancomycin resistant Enterococcus (VRE), carbapenem resistant Enterobacteriaceae (CRE), or non-resistant bacteria, including Mycobacterium tuberculosis, Streptococcus pneumoniae, Haemophilus influenzae, Moraxella catarrhalis, Mycoplasma, Staphylococcus aureus, Pseudomonas, or Acinetobacter baumannii.

In some embodiments, administration of the oligosaccharide composition reduces the number of inflammatory episodes (e.g., AECOPD episodes or asthma attacks), e.g., associated with viral infections, including, e.g., infections with respiratory viruses. In some embodiments, administration of the oligosaccharide composition reduces the number of inflammatory episodes (e.g., AECOPD episodes or asthma attacks), e.g., associated with viral infections, including, e.g., infections with influenza (FLU), human rhinovirus (HRV), respiratory syncytial virus (RSV) or coronaviruses (Family Coronaviridae).

In some embodiments, following administration of the oligosaccharide composition, the subject experiences fewer inflammatory episodes (e.g., AECOPD episodes or asthma attacks) relative to a control subject (e.g., untreated subject) or a baseline measurement. In some embodiments, following administration of the oligosaccharide composition, the subject experiences less severe inflammatory episodes (e.g., AECOPD episodes or asthma attacks) relative to a control subject (e.g., untreated subject) or a baseline measurement. In some embodiments, the severity of an inflammatory episode is determined by the length of a treatment course and/or the recovery time for the subject. In some embodiments, following administration of the oligosaccharide composition, the subject experiences fewer inflammatory episodes (e.g., AECOPD episodes or asthma attacks) that require hospitalization relative to a control subject (e.g., untreated subject) or a baseline measurement. In some embodiments, administration of the oligosaccharide composition to the subject results in fewer days of hospitalization per year resulting from inflammatory episodes (e.g., AECOPD episodes or asthma attacks) relative to a control subject (e.g., untreated subject) or a baseline measurement.

In some embodiments, following administration of the oligosaccharide composition, the subject experiences a shorter recovery period after an inflammatory episode (e.g., AECOPD episodes or asthma attacks) relative to a control subject (e.g., untreated subject) or a baseline measurement.

In some embodiments, the oligosaccharide composition reduces the levels of pro-inflammatory cytokines in the subject (e.g., in the lungs, sputum and/or gut of the subject). In some embodiments, the pro-inflammatory cytokines are selected from the group consisting of IL-8, IL-1, IL-10, IL-33, IL-6, IP-10, TSLP, neutrophil elastase, lactoferrin and eosinophil cationic protein (ECP).

In some embodiments, the oligosaccharide composition promotes increased levels of anti-inflammatory cytokines in the subject (e.g., in the lungs, sputum and/or gut of the subject). In some embodiments, the anti-inflammatory cytokines are selected from the group consisting of IL-4, IL-10 and IL-13.

In some embodiments, the oligosaccharide composition reduces the relative or absolute abundance of neutrophils and/or eosinophils in the subject (e.g., in the lungs and/or sputum of the subject). In some embodiments, the oligosaccharide composition reduces microvascular leakage. Microvascular leakage is associated with inflammation and is thought to play a critical role in asthma. Microvascular leakage produces plasma exudation and thickening of the bronchial mucosa which may underlie airway hyperresponsiveness. In some embodiments, microvascular leakage leads to increased vascular permeability and loss of proteins and small molecule across the vasculature. In some embodiments, the oligosaccharide reduces the levels of alpha-2-macroglobulin in sputum of the subject.

In some embodiments, the subject has at least one comorbidity condition. In some embodiments, the comorbidity condition is selected from the group consisting of diabetes mellitus, cardiovascular disease, hypertension, renal disease, liver disease, an immunocompromised condition, cancer, a neurologic disorder and stroke. In some embodiments, the subject is at least 45 years old. In some embodiments, the subject is overweight or obese. In some embodiments, the subject has a body mass index (BMI) of at least 25, at least 30, or at least 35.

In some embodiments, the subject is at risk of a bacterial infection, optionally wherein the bacterial infection is an infection of the gastrointestinal tract, lungs, bloodstream, central nervous system, lymphatic system, or soft tissues. In some embodiments, the bacterial infection is an infection caused by a vancomycin resistant Enterococcus (VRE), carbapenem resistant Enterobacteriaceae (CRE), or non-resistant bacteria, including Mycobacterium tuberculosis, Streptococcus pneumoniae, Haemophilus influenzae, Moraxella catarrhalis, Mycoplasma, Staphylococcus aureus, Pseudomonas, or Acinetobacter baumannii.

In some embodiments, following administration of the oligosaccharide composition, the subject experiences a reduction of the severity of one or more symptoms of the inflammatory lung disease or disorder relative to a control subject (e.g., untreated subject) or a baseline measurement. In some embodiments, one or more symptoms are selected from the group consisting of: persistent cough, coughs that include excess mucus, shortness of breath, wheezing or squeaking while breathing, chest tightness, frequent colds or bacterial infections, low energy, sudden weight loss, and swelling of extremities (e.g., ankles, feet, legs). In some embodiments, the subject experiences a reduction of the severity of 2, 3, 4, or 5 symptoms.

In some embodiments, the oligosaccharide composition promotes increased relative or absolute abundance of anti-inflammatory metabolites in the subject (e.g., in the lungs, sputum and/or gut of the subject) relative to a baseline measurement or control. In some embodiments, the anti-inflammatory metabolites are short-chain fatty acids (SCFAs), indoles, tryptophan and derivatives thereof, and/or small molecule modulators of Aryl Hydrocarbon Receptor (AHR) and/or IL-22. In some embodiments, the oligosaccharide composition promotes increased relative or absolute abundance of short-chain fatty acids in the subject (e.g., in the lungs, sputum and/or gut of the subject) relative to a baseline measurement or control. In some embodiments, the oligosaccharide composition promotes increased relative or absolute abundance of short-chain fatty acids by at least 2-fold (e.g., at least 3-fold, at least 4-fold, at least 5-fold) in an ex vivo fecal sample collected from the subject relative to a baseline measurement or control. In some embodiments, the short-chain fatty acids comprise propionate, butyrate and/or acetate. In some embodiments, the amount of propionate is increased by at least 1.2 fold (e.g., at least 1.2, 1.3, 1.4, 1.5, 1.75, 2, 2.25, 2.5, 2.75, 3, 3.25, 3.5, 3.75, 4, 4.25, 4.5, 4.75, or 5 fold) in an ex vivo fecal sample collected from the subject relative to a baseline measurement or control. In some embodiments, the relative amounts of acetate, propionate, and butyrate produced (e.g., in the gastrointestinal tract and/or an ex vivo fecal sample treated with the oligosaccharide composition) are about 40-70% acetate, about 30-50% propionate, and about 5-20% butyrate. In some embodiments, the relative increase of the amount of propionate in the ex vivo fecal sample collected from the subject is substantially the same as the relative increase of the amount of propionate in ex vivo fecal samples collected from other subjects receiving administration of the oligosaccharide composition (e.g., relative increase for the subject is within +50% of the relative increase for other subjects).

In some embodiments, the oligosaccharide composition is produced from a mixture comprising 30-60% dextrose monomers, 30-60% galactose monomers, and 5-15% mannose monomers. In some embodiments, the oligosaccharide composition is produced from a mixture comprising about 45% dextrose monomers, about 45% galactose monomers, and about 10% mannose monomers.

In some embodiments, the mixture further comprises an acid catalyst and is heated at a temperature in a range of 100° C. to 160° C.

In some embodiments, the oligosaccharide composition has an average degree of polymerization of 8-15.

In some embodiments, a method described herein comprises administering the oligosaccharide composition to the intestines (e.g., the large intestine). In some embodiments, the oligosaccharide composition is self-administered to the subject. In some embodiments, the oligosaccharide composition is formulated as a pharmaceutical composition for oral delivery In some embodiments, the oligosaccharide composition is orally administered to the subject. In some embodiments, the oligosaccharide composition is administered to the subject once per day or twice per day. In some embodiments, the oligosaccharide composition is administered in combination with a standard-of-care treatment.

In some embodiments, the oligosaccharide composition is administered in combination with a population of probiotic or commensal bacteria. In some embodiments, the oligosaccharide composition is administered in combination with a population of Lactobacillus or Akkermansia.

In some embodiments, an oligosaccharide composition comprises a plurality of oligosaccharides, the plurality of oligosaccharides being characterized by a multiplicity-edited gradient-enhanced ¹H-¹³C heteronuclear single quantum correlation (HSQC) NMR spectrum comprising signals 5, 6, 7, and 15 of the following table, wherein the area under the curve (AUC) for each of signals 1-15 is determined by obtaining the integration of integral regions defined by an 1H center position and an 13C center position using an elliptical shape, and wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, an oligosaccharide composition is characterized the oligosaccharide composition is characterized by a multiplicity-edited gradient-enhanced ¹H-¹³C heteronuclear single quantum correlation (HSQC) NMR spectrum comprising signals 5, 6, 7, 10, 14, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, an oligosaccharide composition is characterized by a multiplicity-edited gradient-enhanced ¹H-¹³C heteronuclear single quantum correlation (HSQC) NMR spectrum comprising signals 5, 6, 7, and 10-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some aspects, the disclosure provides an oligosaccharide composition comprising a plurality of oligosaccharides that are minimally digestible in humans, the composition being characterized by a multiplicity-edited gradient-enhanced ¹H-¹³C heteronuclear single quantum correlation (HSQC) NMR spectrum comprising signals 5, 6, 7, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising signals 5, 6, 7, 10, 14, and 15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising signals 5, 6, 7, and 10-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising signals 1-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, the oligosaccharide composition is characterized by a multiplicity-edited gradient-enhanced ¹H-¹³C heteronuclear single quantum correlation (HSQC) NMR spectrum comprising any combination of signals 1-15 (e.g., signals 5, 6, 7, and 15) of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.18-27.11 |
| 2 | 3.75 | 66.06 | 3.31-6.10 |
| 3 | 3.97 | 66.15 | 2.37-3.69 |
| 4 | 3.96 | 69.28 | 0.42-4.72 |
| 5 | 3.96 | 70.62 | 8.43-11.69 |
| 6 | 3.92 | 71.26 | 1.09-3.10 |
| 7 | 3.55 | 71.34 | 4.01-6.37 |
| 8 | 3.97 | 71.56 | 2.77-4.29 |
| 9 | 3.72 | 72.35 | 8.51-9.25 |
| 10 | 3.33 | 73.74 | 11.60-12.00 |
| 11 | 4.06 | 77.34 | 3.87-4.22 |
| 12 | 4.11 | 81.59 | 3.27-3.57 |
| 13 | 4.96 | 98.7 | 9.90-13.39 |
| 14 | 4.5 | 103.29 | 4.45-6.70 |
| 15 | 4.44 | 103.86 | 1.60-2.63 |

In some embodiments, the oligosaccharide composition is characterized by a multiplicity-edited gradient-enhanced ¹H-¹³C heteronuclear single quantum correlation (HSQC) NMR spectrum comprising any combination of signals 1-15 (e.g., signals 5, 6, 7, and 15) of the following table, wherein the spectrum is generated using a sample of the oligosaccharide composition having less than 2% monomer.

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 19.49-27.81 |
| 2 | 3.75 | 66.06 | 3.04-6.36 |
| 3 | 3.97 | 66.15 | 2.23-3.83 |
| 4 | 3.96 | 69.28 | 0.00-5.15 |
| 5 | 3.96 | 70.62 | 8.10-12.02 |
| 6 | 3.92 | 71.26 | 0.88-3.32 |
| 7 | 3.55 | 71.34 | 3.77-6.61 |
| 8 | 3.97 | 71.56 | 2.61-4.45 |
| 9 | 3.72 | 72.35 | 5.98-9.54 |
| 10 | 3.33 | 73.74 | 10.34-12.14 |
| 11 | 4.06 | 77.34 | 2.93-4.33 |
| 12 | 4.11 | 81.59 | 2.54-3.66 |
| 13 | 4.96 | 98.7 | 9.55-13.75 |
| 14 | 4.5 | 103.29 | 4.22-6.94 |
| 15 | 4.44 | 103.86 | 1.51-2.71 |

In some embodiments, an oligosaccharide composition is characterized by a multiplicity-edited gradient-enhanced ¹H-¹³C heteronuclear single quantum correlation (HSQC) NMR spectrum comprising signals 1-15 of the following table, wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, an oligosaccharide composition is characterized by a multiplicity-edited gradient-enhanced ¹H-¹³C heteronuclear single quantum correlation (HSQC) NMR spectrum wherein signals 1-15 are each further characterized by an ¹H integral region and a ¹³C integral region, defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | From | To | Position | from | to |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 10 | 3.33 | 3.27 | 3.4 | 73.74 | 73.26 | 74.22 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |
| 14 | 4.5 | 4.47 | 4.54 | 103.29 | 102.87 | 103.70 |
| 15 | 4.44 | 4.41 | 4.46 | 103.86 | 103.56 | 104.15 |

In some embodiments, an NMR spectrum is obtained by subjecting a sample of the composition to a multiplicity-edited gradient-enhanced ¹H-¹³C heteronuclear single quantum coherence (HSQC) experiment using an echo-antiecho scheme for coherence selection using the following pulse sequence diagram, acquisition parameters and processing parameters:

### Acquisition Parameters

¹H Carrier Frequency=4 ppm
¹³C Carrier Frequency=65 ppm
Number of points in acquisition dimension=596
Spectral range in acquisition dimension=6.23 ppm to 1.83 ppm
Number of points in indirect dimension=300 complex points
Spectral range in indirect dimension=120 ppm to 10 ppm
Recycle delay=1 second
One-bond ¹H-¹³C coupling constant=J_{CH}=146 Hz
Number of scans=8
Temperature=298-299 K
Solvent=D₂O

### Processing Parameters

Window function in direct dimension=Gaussian broadening, 7.66 Hz
Window function in indirect dimension=Gaussian broadening 26.48 Hz
Processing=512 complex points in direct dimension, 1024 complex points in indirect dimension

In some embodiments, an NMR spectrum is obtained by subjecting a sample of the oligosaccharide composition to HSQC NMR, wherein the sample is dissolved in D2O.

In some embodiments, an oligosaccharide composition has been subjected to a de-monomerization procedure.

In some aspects, the disclosure provides an oligosaccharide composition comprising a plurality of oligosaccharides that are minimally digestible in humans, each oligosaccharide comprising a plurality of monomer radicals;

the plurality of oligosaccharides comprising two or more types of monomer radicals selected from radicals (1)-(43):
(1) t-mannopyranose monoradicals, representing 3.0-4.1 mol % of monomer radicals in the plurality of oligosaccharides;
(2) t-glucopyranose monoradicals, representing 13.6-17.6 mol % of monomer radicals in the plurality of oligosaccharides;
(3) t-galactofuranose monoradicals, representing 3.0-4.2 mol % of monomer radicals in the plurality of oligosaccharides;
(4) t-glucofuranose monoradicals, representing 0.1-0.7 mol % of monomer radicals in the plurality of oligosaccharides;
(5) t-galactopyranose monoradicals, representing 9.7-11.7 mol % of monomer radicals in the plurality of oligosaccharides;
(6) 3-glucopyranose monoradicals, representing 3.8-4.6 mol % of monomer radicals in the plurality of oligosaccharides;
(7) 2-mannopyranose and/or 3-mannopyranose monoradicals, representing 0.8-2.0 mol % of monomer radicals in the plurality of oligosaccharides;
(8) 2-glucopyranose monoradicals, representing 2.7-3.0 mol % of monomer radicals in the plurality of oligosaccharides;
(9) 2-galactofuranose and/or 2-glucofuranose monoradicals and/or 3-glucofuranose, representing 0.8-1.8 mol % of monomer radicals in the plurality of oligosaccharides;
(10) 3-galactopyranose monoradicals, representing 2.8-3.8 mol % of monomer radicals in the plurality of oligosaccharides;
(11) 3-galactofuranose monoradicals, representing 1.6-2.2 mol % of monomer radicals in the plurality of oligosaccharides;
(12) 6-mannopyranose monoradicals, representing 2.1-2.5 mol % of monomer radicals in the plurality of oligosaccharides;
(13) 2-galactopyranose monoradicals, representing 1.7-2.4 mol % of monomer radicals in the plurality of oligosaccharides;
(14) 6-glucopyranose monoradicals, representing 9.5-11.1 mol % of monomer radicals in the plurality of oligosaccharides;
(15) 4-galactopyranose and/or 5-galactofuranose monoradicals, representing 2.5-3.1 mol % of monomer radicals in the plurality of oligosaccharides;
(16) 4-glucopyranose and/or 5-glucofuranose and/or 6-mannofuranose monoradicals, representing 3.0-3.9 mol % of monomer radicals in the plurality of oligosaccharides;
(17) 2,3-galactofuranose diradicals, representing 0.1-0.4 mol % of monomer radicals in the plurality of oligosaccharides;
(18) 6-glucofuranose monoradicals, representing 0.1-0.8 mol % of monomer radicals in the plurality of oligosaccharides;
(19) 6-galactofuranose monoradicals, representing 2.3-2.7 mol % of monomer radicals in the plurality of oligosaccharides;
(20) 6-galactopyranose monoradicals, representing 7.1-8.8 mol % of monomer radicals in the plurality of oligosaccharides;
(21) 3,4-galactopyranose and/or 3,5-galactofuranose and/or 2,3-galactopyranose diradicals, representing 0.9-1.1 mol % of monomer radicals in the plurality of oligosaccharides; (22) 3,4-glucopyranose and/or 3,5-glucofuranose diradicals, representing 0.5-0.8 mol % of monomer radicals in the plurality of oligosaccharides;
(23) 2,3-glucopyranose diradicals, representing 0.1-2.1 mol % of monomer radicals in the plurality of oligosaccharides;
(24) 2,4-mannopyranose and/or 2,5-mannofuranose diradicals, representing 0.1-0.9 mol % of monomer radicals in the plurality of oligosaccharides;
(25) 2,4-glucopyranose and/or 2,5-glucofuranose and/or 2,4-galactopyranose and/or 2,5-galactofuranose diradicals, representing 0.5-1.9 mol % of monomer radicals in the plurality of oligosaccharides;
(26) 4,6-mannopyranose and/or 5,6-mannofuranose diradicals, representing 0.4-0.7 mol % of monomer radicals in the plurality of oligosaccharides;
(27) 3,6-glucopyranose diradicals, representing 2.0-2.9 mol % of monomer radicals in the plurality of oligosaccharides;
(28) 3,6-mannopyranose diradicals, representing 0.4-0.7 mol % of monomer radicals in the plurality of oligosaccharides;
(29) 2,6-mannopyranose diradicals, representing 0.4-0.5 mol % of monomer radicals in the plurality of oligosaccharides;
(30) 3,6-glucofuranose diradicals, representing 0.1-0.3 mol % of monomer radicals in the plurality of oligosaccharides;
(31) 2,6-glucopyranose and/or 4,6-glucopyranose and/or 5,6-glucofuranose diradicals, representing 1.7-2.6 mol % of monomer radicals in the plurality of oligosaccharides;
(32) 3,6-galactofuranose diradicals, representing 0.9-1.2 mol % of monomer radicals in the plurality of oligosaccharides;
(33) 4,6-galactopyranose and/or 5,6-galactofuranose diradicals, representing 2.1-2.9 mol % of monomer radicals in the plurality of oligosaccharides;
(34) 3,6-galactopyranose diradicals, representing 2.0-2.7 mol % of monomer radicals in the plurality of oligosaccharides;
(35) 2,6-galactopyranose diradicals, representing 1.0-1.5 mol % of monomer radicals in the plurality of oligosaccharides;
(36) 3,4,6-mannopyranose and/or 3,5,6-mannofuranose and/or 2,3,6-mannofuranose triradicals, representing 0.1 mol % of monomer radicals in the plurality of oligosaccharides;
(37) 3,4,6-galactopyranose and/or 3,5,6-galactofuranose and/or 2,3,6-galactofuranose triradicals, representing 0.5-1.0 mol % of monomer radicals in the plurality of oligosaccharides;
(38) 3,4,6-glucopyranose and/or 3,5,6-glucofuranose triradicals, representing 0.1-0.6 mol % of monomer radicals in the plurality of oligosaccharides;
(39) 2,3,6-mannopyranose triradicals, representing 0.1-0.3 mol % of monomer radicals in the plurality of oligosaccharides;
(40) 2,4,6-glucopyranose and/or 2,5,6-glucofuranose triradicals, representing 0.1-0.8 mol % of monomer radicals in the plurality of oligosaccharides;
(41) 2,3,6-galactopyranose and/or 2,4,6-galactopyranose and/or 2,5,6-galactofuranose triradicals, representing 0.1-1.3 mol % of monomer radicals in the plurality of oligosaccharides;
(42) 2,4,6-galactopyranose and/or 2,5,6-galactofuranose triradicals, representing 0.1-0.9 mol % of monomer radicals in the plurality of oligosaccharides;
(43) 2,3,6-glucopyranose triradicals, representing 0.1-0.7 mol % of monomer radicals in the plurality of oligosaccharides;
the oligosaccharide composition comprising at least one glucofuranose or glucopyranose radical, at least one mannofuranose or mannopyranose radical, and at least one galactofuranose or galactopyranose radical.

In some embodiments, an oligosaccharide composition comprises at least one glucofuranose or glucopyranose radical, at least one manofuranose or manopyranose radical, and at least one galactofuranose or galactopyranose radical.

In some embodiments, the molar percentages of the monomer radicals are determined using a permethylation assay.

In some embodiments, an oligosaccharide composition is produced by a process comprising:
(a) forming a reaction mixture comprising dextrose monomer, galactose monomer, and mannose monomer wherein the molar ratio of dextrose to galactose is about 1:1 and the molar ratio of dextrose to mannose is about 4.5:1 with an acid catalyst comprising positively charged hydrogen ions; and
(b) promoting acid catalyzed oligosaccharide formation in the reaction mixture by transferring sufficient heat to the reaction mixture to maintain the reaction mixture at its boiling point.

In some embodiments, step (b) comprises promoting acid catalyzed oligosaccharide formation in the reaction mixture by transferring sufficient heat to the reaction mixture to maintain the reaction mixture at its boiling point until the weight percent of total monomer content in the oligosaccharide composition is in a range of 2% to 20%, wherein the total monomer content comprises dextrose monomer, galactose monomer, and/or mannose monomer. In some embodiments, step (b) comprises loading the preparation with an acid catalyst comprising positively charged hydrogen ions, in an amount such that the molar ratio of positively charged hydrogen ions to total dextrose monomer, galactose monomer, and mannose monomer content is in an appropriate range.

In some embodiments, steps (a) and (b) occur simultaneously.

In some embodiments, step (a) comprises heating the reaction mixture under agitation conditions to a temperature in a range of 100° C. to 160° C. In some embodiments, step (a) comprises heating the reaction mixture under agitation conditions to a temperature in a range of 135° C. to 145° C. In some embodiments, step (a) comprises heating the reaction mixture under agitation conditions at a temperature in a range of 100° C. to 160° C. In some embodiments, step (a) comprises heating the reaction mixture under agitation conditions at a temperature in a range of 135° C. to 145° C. In some embodiments, step (a) comprises gradually increasing the temperature (e.g., from room temperature) to about 140° C., under suitable conditions to achieve homogeneity and uniform heat transfer.

In some embodiments, step (b) comprises maintaining the reaction mixture at atmospheric pressure or under vacuum, at a temperature in a range of 135° C. to 145° C., under conditions that promote acid catalyzed oligosaccharide composition formation, until the weight percent of dextrose monomer, galactose monomer, and mannose monomer in the oligosaccharide composition is in a range of 4-14. In some embodiments, step (b) comprises gradually increasing the temperature (e.g., from room temperature) to about 140° C., under suitable conditions to achieve homogeneity and uniform heat transfer.

In some embodiments, said heating comprises melting the preparation and/or heating the preparation under suitable conditions to achieve homogeneity and uniform heat transfer. In some embodiments, the acid catalyst is a soluble catalyst. In some embodiments, the soluble catalyst is an organic acid, optionally a weak organic acid. In some embodiments, the acid catalyst is citric acid, acetic acid, or propionic acid. In some embodiments, the acid catalyst is a strong acid cation exchange resin having one or more physical and chemical properties according to Table 1 and/or wherein the catalyst comprises >3.0 mmol/g sulfonic acid moieties and <1.0 mmol/gram cationic moieties.

In some embodiments, an oligosaccharide composition comprises a mean degree of polymerization is in a range of 5-20, 6-18, 7-15.5, 8-15, 9-15, 10-15, or 11-15. In some embodiments, an oligosaccharide composition comprises mean degree of polymerization is in a range of 11-15.

In some embodiments, the composition has a MWw (g/mol) in a range of 1905-2290. In some embodiments, the composition has a MWw (g/mol) in a range of 1740-2407. In some embodiments, the composition has a MWw (g/mol) in a range of 1863-2268. In some embodiments, the composition has a MWw (g/mol) in a range of 1700-2295. In some embodiments, the composition has a MWn (g/mol) in a range of 1033-1184. In some embodiments, the composition has a MWn (g/mol) in a range of 975-1155. In some embodiments, the composition has a MWn (g/mol) in a range of 984-1106. In some embodiments, the composition has a MWn (g/mol) in a range of 938-1120.

In some embodiments, a solution comprising the oligosaccharide composition has a pH in a range of 2.50-7.00, optionally 2.50-3.50.

In some embodiments, the composition comprises oligomers having two or more repeat units (DP2+) in a range of 86-96 weight percent. In some embodiments, the composition comprises oligomers having two or more repeat units (DP2+) in a range of 81-100 weight percent. In some embodiments, the composition comprises oligomers having at least three linked monomer units (DP3+) in a range of 85-90 weight percent.

In some embodiments, the composition further comprises: 0.18-0.51% w/w levoglucosan. 0.01-0.05% w/w lactic acid, and/or 0.04-0.07% w/w formic acid.

In some embodiments, the composition further comprises: 0.40-0.53% w/w levoglucosan, 0.01-0.02% w/w lactic acid, 0.01-0.04% w/w formic acid, and/or 0.01-0.04% w/w citric acid.

### BRIEF DESCRIPTION OF THE DRAWINGS

FIGS. 1A-1B provide graphs showing the ability of the selected oligosaccharide composition to produce an increase in concentration of short-chain fatty acids (acetate, propionate, butyrate) in fecal samples from seven healthy human subjects relative to a negative control (water). FIG. 1A shows the amounts (in mM) of acetate, propionate, and butyrate produced in fecal samples incubated with either the selected oligosaccharide composition or water. FIG. 1B shows the fold change in total concentration of short-chain fatty acids (SCFA) in fecal samples incubated with the selected oligosaccharide composition relative to the same fecal samples incubated with water.

FIG. 2 provides graphs showing the amounts (in mM) of acetate, propionate, and butyrate produced in fecal samples incubated with either the selected oligosaccharide composition or a commercial oligosaccharide selected from xylooligosaccharides (XOS), polydextrose (PDX), galactooligosaccharides (GOS), and fructooligosaccharides (FOS). The selected oligosaccharide provides highly consistent and robust results across the three tested fecal communities, particularly in comparison to the commercial oligosaccharides.

FIG. 3 provides a HSQC NMR spectra of the selected oligosaccharide composition.

FIG. 4 shows tables of healthcare utilization of study participants (overall population at top, patients with at least one comorbidity at bottom) treated with SSC and oligosaccharide composition or SSC alone.

FIG. 5A shows a Kaplan-Meier plot showing proportion of subjects with resolution of symptoms associated with lung disease and/or inflammation over time measured by analysing 13 symptoms, wherein the subjects were at least 45 years old or having at least one comorbidity, and were treated with either SSC and oligosaccharide composition or SSC alone.

FIG. 5B shows a Kaplan-Meier plot showing proportion of subjects with resolution of symptoms associated with lung disease and/or inflammation over time measured by analysing 13 symptoms, wherein the subjects were at least 45 years old or having a BMI of at least 35, and were treated with either SSC and oligosaccharide composition or SSC alone.

FIG. 5C shows a Kaplan-Meier plot showing proportion of subjects with resolution of symptoms associated with lung disease and/or inflammation over time measured by analysing 13 symptoms, wherein the subjects were at least 45 years old or having at least one high-risk comorbidity, and were treated with either SSC and oligosaccharide composition or SSC alone.

FIG. 6 shows annotation of a ¹H-¹³C gHSQC NMR spectra of the selected oligosaccharide composition, assigning particular bonds of the oligosaccharide composition to each peak. Annotations indicate glycan locations, not total peak volumes. The exemplary selected oligosaccharide composition examined included approximately 13% monomer content.

FIG. 7 depicts an HSQC NMR pulse sequence diagram.

FIG. 8 provides graphs showing reductions in pathogen growth (Enterobacteriaceae and Enterococcaceae) and an increase in commensal growth (Parabacteroides) in fecal samples spiked with vancomycin-resistant Enterococcaceae.

FIG. 9 provides graphs showing the ability of the selected oligosaccharide composition to support the growth of single strains of commensal bacteria (Parabacteroides and Bacteroides).

FIG. 10 provides plots showing plasma concentrations of TNF-alpha and IFN-gamma in subjects treated with either SSC and oligosaccharide composition or SSC alone.

### DETAILED DESCRIPTION OF INVENTION

Aspects of the disclosure relate to oligosaccharide compositions that are effective for treating and preventing inflammatory lung diseases and disorders (e.g., chronic obstructive pulmonary disease (COPD)) in a subject. The intestinal microbiome is a complex ecosystem that integrates environmental inputs, such as diet, with genetic and immune signals to affect a subject's metabolism, immunity and response to infection. Aspects of the disclosure relate, at least in part, to a recognition that beyond the local gut immune regulation by the resident microbiota, long-reaching immuno-modulatory impact in other organs, including the pulmonary immune system, is associated with the gut microbiota.

In embodiments, modulation of the gut microbiome of a subject (e.g., a human subject), as described herein, provides an opportunity to have a beneficial impact towards reducing the progression and/or severity of inflammatory lung diseases and disorders such as COPD.

The microbiome residing in the human gut plays a role in modulating systemic immunity and inflammatory responses in distal organs and body-sites including the lung. The “gut-lung axis” is thought to play a role in chronic respiratory diseases as well. Case and epidemiology studies involving COPD patients, associate greater intake of dietary fiber with reduced COPD risk, better lung function and reduced respiratory symptoms (Varraso et al. 2010 Am J Epidemiol.; Fonseca Wald et al. 2014. Respirol.). Further, the fecal microbiome and metabolome have a distinct signature in COPD patients (Bowerman et al. 2020 Nat. Commun.; Chiu et al. 2021 PLOS One). It has been shown that Lactobacillus spp. culture supernatants block >50% of adhesion in vitro by Moraxella to human airway epithelial cells and reduce pro-inflammatory cytokines (van den Broek et al. 2018. Benef Microbes). It has been suggested that high fiber diets might attenuate emphysema development in mice (Jang et al. Sci Rep. 2021 11 (1): 7008). Epidemiology studies demonstrate that maternal diet and bacterial metabolites might affect the occurrence of allergic asthma in offspring (Thorburn et al. 2015 Nat. Commun.). Multiple in vivo studies show that targeted probiotics such as Akkermansia (Michalovich et al. 2019 Nat Commun) or dietary supplementation with SCFAs (Cait et al. 2017 Mucosal Immunology) ameliorate asthma susceptibility and lung inflammation.

Acute respiratory viral infectious diseases such as COVID-19 share several disease aspects with chronic non-infectious inflammatory diseases such as COPD, including that both diseases: (i) are associated with respiratory pathogens (COVID-19 with SARS-CoV-2, COPD with respiratory viruses (influenza, rhinovirus, respiratory syncytial virus) and bacteria (Moraxella, Haemophilus, Streptococcus. pneumoniae, Staphylococcus aureus and Pseudomonas aeruginosa), e.g., pathogens associated with a majority of severe acute exacerbations in COPD (AECOPD)); (ii) exhibit (in some embodiments, extensive) lung damage, e.g., due to hyperactivation of the immune response and over-stimulation of pro-inflammatory host factors; (iii) are associated with (in some embodiments, rapid) decline in lung function; (iv) are associated with increased risk of death and requirement of hospitalization and/or urgent care, in severe cases; (v) are associated with a risk of secondary bacterial infections; (vi) are associated with comorbidities (which are, e.g., associated with worse disease states and/or disease progression and/or longer recovery times, e.g., from infections or exacerbations). Further, COPD and COVID-19 share over-activation of multiple cytokine-signaling pathways (e.g., (IL-33, CXCL8, TNF-α, IL-10, IFN-γ) and other immune modulators (elevated eosinophils and neutrophils-infiltration) which is, in some embodiments, associated with severe and, in some embodiments, irreversible lung tissue damage.

In some embodiments, oligosaccharide compositions described herein (e.g., orally administered, non-absorbed synthetic oligosaccharide compositions) that modulate the composition and/or metabolic output of the gut microbiome are useful in treating inflammatory lung diseases and disorders, such as, e.g., COPD and asthma. In some embodiments, selected oligosaccharide compositions of the disclosure provide modulation over the taxonomic composition of the gut microbiome and its metabolic output, including the absolute quantities and relative abundance of SCFAs and other metabolites. Specifically, in some embodiments, selected oligosaccharide compositions promote beneficial SCFA profiles in the gut and/or growth of bacterial taxa that support the ability of a subject to mount an appropriate immune and inflammatory response while, in some embodiments, also preventing an over-aggressive response such as, for example, a cytokine storm.

A cytokine storm (also known as hypercytokinemia), in some embodiments, involves an immune reaction in which the body releases too many cytokines into the blood too quickly (e.g., at the same time). The release of a large amount of cytokines at one time can be harmful. In some embodiments, a cytokine storm is characterized by high fever, inflammation (e.g., redness and swelling), severe fatigue and/or nausea. In some embodiments, a cytokine storm is severe or life threatening and/or can lead to multiple organ failure.

In some embodiments, selected oligosaccharide compositions are not significantly digested by the limited number of human carbohydrate-modifying enzymes in the small intestine and pass into the large intestine where they can be enzymatically digested by a repertoire of carbohydrate-active enzymes produced by a community of resident commensal bacteria (e.g., Parabacteroides and Bacteroides). In some embodiments, major products of digestion of selected oligosaccharide composition by the gut bacteria are SCFAs (e.g., butyrate, propionate, and acetate). In some embodiments, these SCFAs serve a role in the maintenance of healthy gut epithelial function. In some embodiments, butyrate, for example, is used as the primary source of energy for gut epithelial cells and promotes maintenance of epithelial integrity. In some embodiments, maintenance of epithelial integrity is important to prevent inappropriate activation of innate and adaptive immune cells. In some embodiments, butyrate activates G-protein coupled receptors (GPCRs) displayed on the surface of epithelial and immune cells, as well as inhibits histone deacetylases in the nucleus of these cell types. In certain embodiments, propionate promotes gut immune homeostasis by activating these same GPCRs. In some embodiments, in addition to producing SCFAs, digestion of the selected oligosaccharide composition results in the preferential growth of commensal bacteria relative to unwanted pathogenic bacteria.

In some embodiments, gut derived metabolites (e.g., SCFAs) and the direct interaction and migration of immune cells from gut to lung by the common mucosal immune system can have a beneficial impact on pulmonary infections (such as caused by respiratory viruses, e.g., coronavirus infections). For example, SCFAs produced by gut bacteria (e.g., resulting from treatment with selected oligosaccharide compositions provided herein) migrate through the circulation (e.g., blood stream) to stimulate immune response in the lung, and, in some cases, different factors from the lung effect immune response in the gastrointestinal tract. Additionally, in some embodiments, immune cells induced by certain antigens move through lymphatic duct between both of these organs leading to modulation of the immune response according to the methods provided herein.

Some methods provided herein result in induction of SCFAs in the gut microbiome. In some embodiments, such SCFAs modulate host inflammation, control adaptive immunity, and/or promote immune tolerance locally in the gut and/or systemically throughout body of the subject. In addition, in some embodiments, SCFAs produced according to methods provided herein influence macrophage functionality to mitigate neutrophil-mediated tissue damage. In some embodiments, this effect is advantageous because inflammatory responses or episodes can elicit a syndrome known as cytokine storm.

In addition to gut microbiome derived metabolites (e.g., SCFAs) influencing peripheral inflammatory responses, direct activation of host immune cells and pathways by microbiota in the gut impacts the progression of pulmonary infections. In some embodiments, a dysbiosis in the gut microbiome community (e.g., exhibited as a loss in overall commensal diversity or pathobiont overgrowth) contribute to unfavorable outcomes in inflammatory lung diseases or disorders. Accordingly, in some embodiments, methods and oligosaccharide compositions provided herein are useful for shifting gut microbiome community activity in a manner that supports or promotes immune responses against recurrence of episodes relating to such diseases or disorders.

Some methods and compositions provided herein modulate an immune response to inflammatory lung diseases or disorders (e.g., COPD) by altering the microbiota of the gut. Without wishing to be bound by theory, the disclosure is directed, in part, to the idea that host metabolites processed by the gut microbiota as well as gut microbiota-secreted metabolites can influence features of a host immune response to inflammatory lung diseases or disorders (e.g., COPD). By altering the microbiota with a method or oligosaccharide composition described herein, a host immune response may be enhanced or attenuated. In some embodiments, the oligosaccharide compositions described herein reduce the frequency, duration and/or severity of inflammatory episodes related to an inflammatory lung disease or disorder (e.g., acute exacerbations of COPD (AECOPD), or asthma attacks). Accordingly, in some embodiments, the oligosaccharide compositions described herein reduce the frequency and duration of hospitalizations resulting from inflammatory episodes related to an inflammatory lung disease or disorder (e.g., acute exacerbations of COPD (AECOPD), or asthma attacks). As such, the oligosaccharide compositions described herein may represent an advantageous modality to treating inflammatory lung diseases or disorders such as COPD where one or more symptoms (or the disease itself) relates to undesirable levels of host immune response and/or specifically targeted treatments are not available.

Further aspects of the disclosure, including a description of defined terms, are provided below.

### I. Definitions

Agitation conditions: As used herein, the term “agitation conditions” refers to conditions that promote or maintain a substantially uniform or homogeneous state of a mixture (e.g., a reaction mixture comprising dextrose monomer, galactose monomer, and mannose monomer) with respect to dispersal of solids (e.g., solid catalysts), uniformity of heat transfer, or other similar parameters. Agitation conditions generally include stirring, shaking, and/or mixing of a reaction mixture. In some embodiments, agitation conditions may include the addition of gases or other liquids into a solution. In some embodiments, agitation conditions are used to maintain substantially uniform or homogenous distribution of a catalyst, e.g., an acid catalyst. In some embodiments, a monosaccharide preparation is heated in the presence of an acid catalyst under suitable conditions to achieve homogeneity and uniform heat transfer in order to synthesize an oligosaccharide composition.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Dextrose monomer: As used herein, the term “dextrose monomer” refers to a D-isomer of a glucose monomer, known as _{D}-glucose. In some embodiments, a dextrose monomer is dextrose monohydrate or 70DS corn syrup.

Effective amount: As used herein, the term “effective amount” refers to an administered amount or concentration of an oligosaccharide composition that is necessary and sufficient to elicit a biological response, e.g., in a subject or patient. In some embodiments, an effective amount of an oligosaccharide composition is capable of modulating, e.g., increasing or decreasing, the processing of a metabolite (e.g., an SCFA). In some embodiments, an effective amount of an oligosaccharide composition is capable of modulating, e.g., increasing or decreasing, the concentration or number of at least one microbial species (e.g., SCFA-producing species). In some embodiments, an effective amount of an oligosaccharide composition is capable of modulating, e.g., increasing or decreasing, the activity or levels of an enzyme in a subject. In some embodiments, an effective amount of an oligosaccharide composition is capable of modulating, e.g., decreasing, the symptoms of a disease (e.g., an inflammatory lung disease). In some embodiments, an effective amount of an oligosaccharide composition is capable of reducing the acquisition of, colonization of, or reducing the reservoir of a pathogen (e.g., a drug or antibiotic resistant pathogen, or an MDR pathogen or a virus (e.g., viral load) in a subject. In some embodiments, an effective amount of an oligosaccharide composition is capable of treating a subject having an inflammatory lung disease or disorder (e.g., COPD or asthma).

Galactose monomer: As used herein, the term “galactose monomer” generally refers to a D-isomer of a galactose monomer, known as _{D}-galactose.

Mannose monomer: As used herein, the term “mannose monomer” generally refers to a D-isomer of a mannose monomer, known as _{D}-mannose.

Monosaccharide Preparation: As used herein, the term “monosaccharide preparation” refers to a preparation that comprises two or more monosaccharides (e.g., dextrose monomer, galactose monomer, and mannose monomer). In some embodiments, a monosaccharide preparation comprises dextrose monomers, galactose monomers, and mannose monomers.

Oligosaccharide: As used herein, the term “oligosaccharide” (which may be used interchangeably with the term “glycan” in some contexts) refers to a saccharide molecule comprising at least two monosaccharides (e.g., dextrose monomers, galactose monomers, mannose monomers) linked together via a glycosidic bond (having a degree of polymerization (DP) of at least 2 (e.g., DP2+)). In some embodiments, an oligosaccharide comprises at least two, at least three, at least four, at least five, at least six, at least seven, at least eight, at least nine, or at least ten monosaccharides subunits linked by glycosidic bonds. In some embodiments, an oligosaccharide is in the range of 3-20, 4-16, 5-15, 8-12, 5-25, 10-25, 20-50, 40-80, or 75-100 monosaccharides linked by glycosidic bonds. In some embodiments, an oligosaccharide comprises at least one 1,2; 1,3; 1,4; and/or 1,6 glycosidic bond. Oligosaccharides may be linear or branched. Oligosaccharides may have one or more glycosidic bonds that are in alpha-configurations and/or one or more glycosidic bonds that are in beta-configurations.

Pharmaceutical Composition: As used herein, a “pharmaceutical composition” refers to a composition having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions or preparations typically meet the microbiological specifications and criteria for non-sterile pharmaceutical products as described in the U.S. Pharmacopeia (USP) or European Pharmacopoeia (EP). Any oligosaccharide composition described herein may be formulated as a pharmaceutical composition.

Subject: As used herein, the term “subject” refers to a human subject or patient. Subjects may include a newborn (a preterm newborn, a full-term newborn), an infant up to one year of age, young children (e.g., 1 yr to 12 yrs), teenagers, (e.g., 13-19 yrs), adults (e.g., 20-64 yrs), and elderly adults (65 yrs and older). In some embodiments, a subject is an elderly adults (65 yrs and older). In some embodiments, a subject is a healthy subject. In some embodiments, a subject has or is suspected of having an inflammatory lung disease or disorder (e.g., COPD or asthma). In some embodiments, a subject has or is suspected of having a chronic inflammatory lung disease or disorder. In some embodiments, a subject has or is suspected of having an acute inflammatory lung disease or disorder. In some embodiments, a subject has or is suspected of having one or more of the following co-morbidities: cardiovascular disease, diabetes, hypertension, cancer, heart disease, hypertension, prior stroke, and chronic kidney disease. In some embodiments, an oligosaccharide composition is administered to a subject 1-14, 3-12, or 5-10 days after an inflammatory episode (e.g., AECOPD). In some embodiments, a subject has a mild clinical presentation. In some embodiments, a subject is able to manage their illness at home (e.g., in an outpatient setting, without hospitalization). A subject may be able to manage their illness at home depending on the clinical presentation, requirement for supportive care, potential risk factors for severe disease, and the ability of the subject to self-isolate at home. In some embodiments, a subject is hospitalized. In some embodiments, a subject having an inflammatory lung disease or disorder (e.g., COPD or asthma) is a candidate for being treated with a ventilator. In some embodiments, a subject has recently been taken off of a ventilator. In some embodiments, a subject is a patient having higher abundance of pathogen relative to a healthy subject, e.g., a subject colonized with a pathogen (e.g., CRE and/or VRE pathogens) in their gastrointestinal tract (e.g., their colon or intestines). In some embodiments, a subject is a patient receiving broad spectrum antibiotics. In some embodiments, the subject is particularly susceptible to pathogen infection, e.g., the subject is critically-ill and/or immunocompromised. In some embodiments, the subject is a patient having a lower abundance of commensal bacteria relative to a healthy subject in their gastrointestinal tract (e.g., their colon or intestines). In some embodiments, the subject has or is suspected of having chronic obstructive pulmonary disease (COPD), asthma, bronchiectasis, cystic fibrosis, pulmonary fibrosis, interstitial lung disease, tuberculosis, or allergy. In some embodiments, the subject has dysbiosis of the lung microbiome. A subject having COPD may have moderate COPD (Global Initiative for Chronic Obstructive Lung Disease (GOLD) Stage II), severe COPD (GOLD Stage III), or very severe COPD (GOLD Stage IV). A subject having COPD may have a Forced Expiratory Volume in the first second of exhalation (FEV1) of less than 80%, or 30%-79%, or less than 49%, or equal to or less than 30%. In some embodiments, the subject has or is diagnosed with a viral infection, e.g., of the respiratory tract. In some embodiments, the subject has or is diagnosed with a bacterial infection, e.g., of the respiratory tract. In some embodiments, the subject has or is diagnosed with an AECOPD associated with an infection, e.g., a viral or bacterial infection. In some embodiments, the subject has or is diagnosed with an AECOPD associated with a non-infectious environmental trigger.

Treatment and Treating: As used herein, the terms “treating” and “treatment” refer to the administration of a composition to a subject to manage or improve one or more symptoms associated with an inflammatory lung disease or disorder. In some embodiments, a symptom resulting from an inflammatory lung disease or disorder is persistent cough, coughs that include excess mucus, shortness of breath, wheezing or squeaking while breathing, chest tightness, frequent colds or bacterial infections, low energy, sudden weight loss, or swelling of extremities (e.g., ankles, feet, legs). In some embodiments, treatment of an inflammatory lung disease or disorder in a subject results in reversal or a “cure” of the disease or disorder. In some embodiments, a subject who has been administered an oligosaccharide composition experiences only mild adverse effects relating to the oligosaccharide composition, e.g., mild gastrointestinal adverse effects. In some embodiments, mild gastrointestinal adverse effects include one or more of diarrhea, abdominal distension, nausea, abdominal pain, or flatulence. In some embodiments, treatment of an inflammatory lung disease or disorder in a subject results in the subject experiencing less severe symptoms. In some embodiments, treatment of an inflammatory lung disease or disorder in a subject prevents the hospitalization of a subject. In some embodiments, treatment of an inflammatory lung disease or disorder in a subject reduces the likelihood that the subject will need to be hospitalized. In some embodiments, treatment of an inflammatory lung disease or disorder in a subject minimizes or prevents progression of the disease (e.g., progression from mild or moderate symptoms to severe symptoms, e.g., such that subject requires further intervention). In some embodiments, treatment of a subject prevents or reduces the severity of a pathogenic infection (e.g., secondary infection of the lungs or the gut). In some embodiments, treatment of an inflammatory lung disease or disorder in a subject reduces inflammation or the likelihood of a cytokine storm in the subject.

### II. Oligosaccharide Compositions

Provided herein are oligosaccharide compositions, their methods of use for promoting the production of SCFAs in a human subject, and their methods of use in treating or preventing inflammatory lung diseases or disorders (e.g., COPD or asthma) in a human subject.

In one aspect, oligosaccharide compositions are provided herein that comprise a plurality of oligosaccharides selected from Formula (I), Formula (II), and Formula (III):

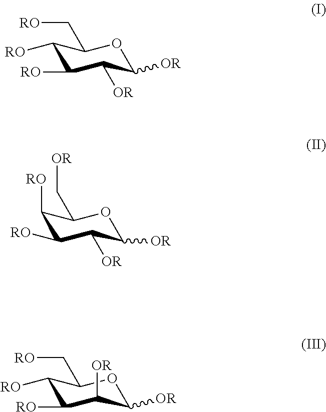

wherein each R independently is selected from hydrogen, and Formulae (Ia), (Ib), (Ic), (Id), (IIa), (IIb), (IIc), (IId), (IIIa), (IIIb), (IIIc), (IIId):

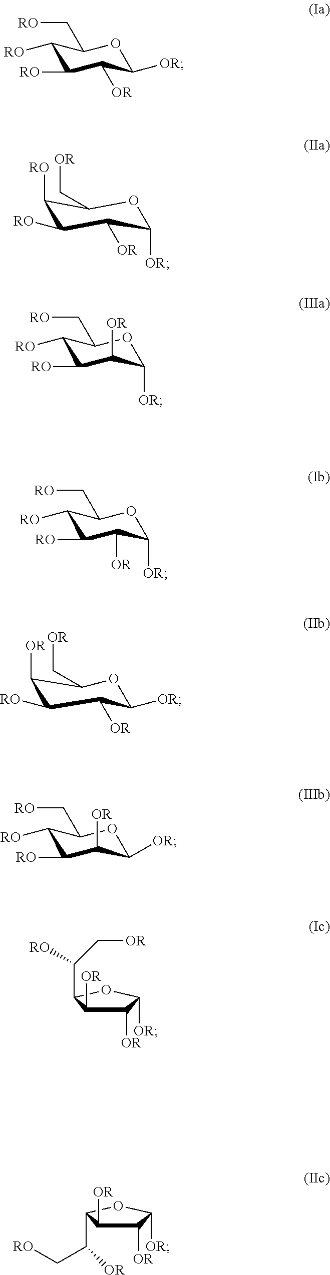

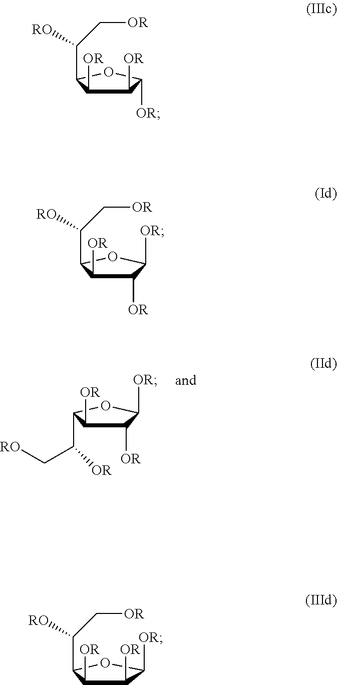

wherein each R independently is as defined above.

In some embodiments, oligosaccharide compositions are produced by a process that initially involves heating a preparation comprising dextrose monomers, galactose monomers, and mannose monomers to a temperature in a range of 100° C. to 160° C., 100° C. to 120° C., 110° C. to 130° C., 120° C. to 140° C., 130° C. to 150° C., or about 140° C. The ratio of dextrose monomers to galactose monomers may be 1:1. The ratio of dextrose monomers to mannose monomers may be 4.5:1. The ratio of galactose monomers to mannose monomers may be 4.5:1. Heating may be performed under agitation conditions. Heating may comprises gradually increasing the temperature (e.g., from room temperature) to about 130° C., about 135° C. about 140° C. about 145° C., or about 150° C. under suitable conditions to achieve homogeneity and uniform heat transfer. An acid catalyst comprising positively charged hydrogen ions is added to the preparation (e.g., following heating). In some embodiments, the acid catalyst is a soluble catalyst. In some embodiments, the acid catalyst is citric acid, acetic acid, or propionic acid. In some embodiments, the acid catalyst is a solid catalyst. In some embodiments, the catalyst is a strong acid cation exchange resin having one or more physical and chemical properties according to Table 1.

TABLE 1
Non-Limiting Example of Strong Acid Cation
Exchange Resin Properties

| Physical Form | | Amber translucent |
|---|---|---|
| | | spherical beads |
| Matrix | | Styrene-DVB, gel |
| Function group | | Sulfonic acid |
| Ionic form as shipped | | H⁺ form |
| Total volume capacity, min. | eq/L | 1.8 |
| | kgr/ft³ as CaCO₃ | 39.3 |
| Moisture retention capacity | % | 50-56 |
| Particle size | | |
| Uniformity coefficient, max. | | 1.1 |
| Harmonic mean diameter | μm | 600 ± 50 |
| Whole uncracked beads | % | 95-100 |
| Total swelling (Na⁺ → H⁺) | % | 8 |
| Particle density | g/mL | 1.2 |
| Shipping density | g/L | 800 |
| | lbs/ft³ | 50 |

In some embodiments, the catalyst comprises >3.0 mmol/g sulfonic acid moieties and <1.0 mmol/gram cationic moieties. In certain embodiments, the catalyst has a nominal moisture content of 45-50 weight percent. In certain embodiments, the catalyst is added at the same time as the dextrose monomers, galactose monomers, and mannose monomers. In some embodiments, after loading of the catalyst with the preparation, the resultant reaction mixture is held at atmospheric pressure and at a temperature in a range of 100° C. to 160° C. 100° C. to 120° C. 110° C. to 130° C., 120° C. to 140° C., 130° C. to 150° C., or about 140° C. under conditions that promote acid catalyzed oligosaccharide formation. In some embodiments, once the weight percent of total monomer content in the oligosaccharide composition (total monomer content comprises the amount of dextrose monomer, galactose monomer, and/or mannose monomer) is in a range of 4-14% (optionally 4-8%, 7-10%, 9-14%, or 12-14%), the reaction mixture is quenched. Quenching typically involves using water (e.g., deionized water) to dilute the reaction mixture, and gradually decrease the temperature of the reaction mixture to 55° C. to 95° C. In some embodiments, the water used for quenching is about 95° C. The water may be added to the reaction mixture under conditions sufficient to avoid solidifying the mixture. In certain embodiments, water may be removed from the reaction mixture by evaporation. In some embodiments, the reaction mixture may contain 93-94 weight percent dissolved solids. Finally, to obtain a purified oligosaccharide composition, the composition is generally separated from the acid catalyst, typically by diluting the quenched reaction mixture with water to a concentration of about 45-55 weight percent and a temperature of below about 85° C. and then passing the mixture through a filter or a series of chromatographic resins. In certain embodiments, the filter used is a 0.45 μm filter. Alternatively, a series of chromatographic resins may be used and generally involves a cationic exchange resin, an anionic exchange resin, and/or a decolorizing polymer resin. In some embodiments, any or all of the types of resins may be used one or more times in any order. In some embodiments, the oligosaccharide composition comprises water at a level below that which is necessary for microbial growth upon storage at room temperature. In certain embodiments, the mean degree of polymerization of all oligosaccharides is in a range of 7-15.5, optionally 11-15. In some embodiments, the oligosaccharide composition comprises water in a range of 45-55 weight percent. In some embodiments, the oligosaccharide composition comprises oligosaccharides that have a MWw (weight-average molecular weight) (g/mol) in a range of 1905-2290. In some embodiments, the oligosaccharide composition comprises oligosaccharides that have a MWn (number-average molecular weight) (g/mol) in a range of 1030-1095. In some embodiments, the oligosaccharide composition has a pH in a range of 2.50-3.50. In some embodiments, the oligosaccharide composition comprises oligomers having two or more repeat units (DP2+) in a range of 86-96 weight percent.

Further, in some embodiments, oligosaccharide compositions may be de-monomerized. In some embodiments, de-monomerization involves the removal of residual saccharide monomers. In some embodiments, de-monomerization is performed using chromatographic resin. Accordingly, in some embodiments, different compositions can be prepared depending upon the percent of monomer present. In some embodiments, oligosaccharide compositions are de-monomerized to a monomer content of about 1%, about 3%, about 5%, about 10%, or about 15%. In some embodiments, oligosaccharide compositions are de-monomerized to a monomer content of about 1-3%, about 3-6%, about 5-8%, about 7-10%, or about 10-15%. In one embodiment, the oligosaccharide compositions is de-monomerized to a monomer content of less than 1%. In one embodiment, the oligosaccharide composition is de-monomerized to a monomer content between about 7% and 10%. In one embodiment, the oligosaccharide compositions is de-monomerized to a monomer content between about 1% and 3%. In one embodiment, de-monomerization is achieved by osmotic separation. In a second embodiment de-monomerization is achieved by tangential flow filtration (TFF). In a third embodiment de-monomerization is achieved by ethanol precipitation.

In some embodiments, oligosaccharide compositions with different monomer contents may also have different measurements for total dietary fiber, moisture, total dietary fiber (dry basis), or percent Dextrose Equivalent (DE). In some embodiments, total dietary fiber is measured according to the methods of AOAC 2011.25. In some embodiments, moisture is measured by using a vacuum oven at 60° C. In some embodiments, total dietary fiber is (dry basis) is calculated. In some embodiments, the percent DE is measured according to the Food Chemicals Codex (FCC).

In some embodiments, the oligosaccharide compositions have a total dietary fiber content of 87.4 percent (on dry basis). In some embodiments, the oligosaccharide compositions have a total dietary fiber content of 81.9-93.0, 82-85, 85-88, 88-90, or 90-93 percent (on dry basis). In some embodiments, the oligosaccharide compositions have a total dietary fiber content of about 82, about 85, about 87, about 90, or about 93 percent (on dry basis). In some embodiments, the oligosaccharide compositions have a total dietary fiber content of 78-97 percent (on dry basis). In some embodiments, the oligosaccharide compositions have a total dietary fiber content of 82-93 percent (on dry basis). In some embodiments, the oligosaccharide compositions have a total dietary fiber content of 14.5-100 percent (on dry basis). In some embodiments, the oligosaccharide compositions have a total dietary fiber content of 34-94 percent (on dry basis).

In some embodiments, the oligosaccharide compositions have a total reducing sugar content (Dextrose Equivalence (DE) (dry solids)) of 6.5-35 percent. In some embodiments, the oligosaccharide compositions have a total reducing sugar content (Dextrose Equivalence (DE) (dry solids)) of 12-29 percent. In some embodiments, the oligosaccharide compositions have a total reducing sugar content (Dextrose Equivalence (DE) (dry solids)) of 5-40, 5-30, 5-25, 10-30, 10-25, 10-20, 15-30, 15-25, or 15-20 percent.

In some embodiments, production of oligosaccharides compositions according to methods provided herein can be performed in a batch process or a continuous process. For example, in one embodiment, oligosaccharide compositions are produced in a batch process, where the contents of the reactor are subjected to agitation conditions (e.g., continuously mixed or blended), and all or a substantial amount of the products of the reaction are removed (e.g., isolated and/or recovered).

In certain embodiments, the methods of using the catalyst are carried out in an aqueous environment. One suitable aqueous solvent is water, which may be obtained from various sources. Generally, water sources with lower concentrations of ionic species (e.g., salts of sodium, phosphorous, ammonium, or magnesium) may be used, in some embodiments, as such ionic species may reduce effectiveness of the catalyst. In some embodiments where the aqueous solvent is water, the water has less than 10% of ionic species (e.g., salts of sodium, phosphorous, ammonium, magnesium). In some embodiments where the aqueous solvent is water, the water has a resistivity of at least 0.1 megaohm-centimeters, of at least 1 megaohm-centimeters, of at least 2 megaohm-centimeters, of at least 5 megaohm-centimeters, or of at least 10 megaohm-centimeters.

In some embodiments, as reactions of methods provided herein progress, water (such as evolved water) is produced with each glycosidic coupling of the one or more saccharide monomer. In certain embodiments, the methods described herein may further include monitoring the amount of water present in the reaction mixture and/or the ratio of water to monomer or catalyst over a period of time. Thus, in some embodiments, the water content of the reaction mixture may be altered over the course of the reaction, for example, removing evolved water produced. Appropriate methods may be used to remove water (e.g., evolved water) in the reaction mixture, including, for example, by evaporation, such as via distillation. In some embodiments, the method comprises including water in the reaction mixture. In certain embodiments, the method comprises removing water from the reaction mixture through evaporation.

In some embodiments, the ratio of dextrose monomer to galactose monomer is about 1:2, 1:1.5, 1:1.4, 1:1.3, 1:1.2, 1:1.1, 1:1, 1.1:1, 1.2:1, 1.3:1, 1.4:1, 1.5:1, or 2:1. In some embodiments, the ratio of dextrose monomer to galactose monomer is about 1:1.

In some embodiments, the ratio of dextrose monomer to mannose monomer is about 1:1, 1.5:1, 2:1, 2.5:1, 3:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, or 5.5:1. In some embodiments, the ratio of dextrose monomer to mannose monomer is about 4.5:1.

In some embodiments, the ratio of galactose monomer to mannose monomer is about 1:1, 1.5:1, 2:1, 2.5:1, 3:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, or 5.5:1. In some embodiments, the ratio of galactose monomer to mannose monomer is about 4.5:1.

In some embodiments, the monosaccharide preparation comprises about 30-60% dextrose monomer, about 30-60% galactose monomer, and 1-25% mannose monomer. In some embodiments, the monosaccharide preparation comprises about 30-60% dextrose monomer, about 30-60% galactose monomer, and about 5-15% mannose monomer. In some embodiments, the monosaccharide preparation comprises about 40-50% dextrose monomer, about 40-50% galactose monomer, and about 5-15% mannose monomer. In some embodiments, the monosaccharide preparation comprises about 45% dextrose monomer, about 45% galactose monomer, and about 10% mannose monomer.

In certain embodiments, the preparation is loaded with an acid catalyst comprising positively charged hydrogen ions. In some embodiments, an acid catalyst is a solid catalyst (e.g., Dowex Marathon C). In some embodiments, an acid catalyst is a soluble catalyst (e.g., citric acid).

In some embodiments, the molar ratio of positively charged hydrogen ions to total dextrose monomer, galactose monomer, and mannose monomer content is in an appropriate range. In some embodiments, the molar ratio of positively charged hydrogen ions to total dextrose monomer, galactose monomer, and mannose monomer content is in a range of 0.01 to 0.1, 0.02 to 0.08, 0.03 to 0.06, or 0.05 to 0.06. In some embodiments, the molar ratio of positively charged hydrogen ions to total dextrose monomer, galactose monomer, and mannose monomer content is in a range of 0.003 to 0.01, 0.005 to 0.02, 0.01 to 0.02, 0.01 to 0.03, 0.02 to 0.03, 0.02 to 0.04, 0.03 to 0.05, 0.03 to 0.08, 0.04 to 0.07, 0.05 to 0.1, 0.05 to 0.2, 0.1 to 0.2, 0.1 to 0.3, or 0.2 to 0.3. In some embodiments, the molar ratio of positively charged hydrogen ions to total dextrose monomer, galactose monomer, and mannose monomer content is in a range of 0.050 to 0.052. In some embodiments, the molar ratio of positively charged hydrogen ions to total dextrose monomer, galactose monomer, and mannose monomer content is in a range of 0.020 to 0.035. In some embodiments, the molar ratio of positively charged hydrogen ions to total dextrose monomer, galactose monomer, and mannose monomer content is 0.028.

In some embodiments, the molar ratio of soluble acid catalyst (e.g., citric acid catalyst) to total dextrose monomer, galactose monomer, and mannose monomer content is in an appropriate range. In some embodiments, the molar ratio of soluble acid catalyst (e.g., citric acid catalyst) to total dextrose monomer, galactose monomer, and mannose monomer content is in a range of 0.01 to 0.1, 0.02 to 0.08, 0.03 to 0.06, or 0.05 to 0.06. In some embodiments, the molar ratio of soluble acid catalyst (e.g., citric acid catalyst) to total dextrose monomer, galactose monomer, and mannose monomer content is in a range of 0.003 to 0.01, 0.005 to 0.02, 0.01 to 0.02, 0.01 to 0.03, 0.02 to 0.03, 0.02 to 0.04, 0.03 to 0.05, 0.03 to 0.08, 0.04 to 0.07, 0.05 to 0.1, 0.05 to 0.2, 0.1 to 0.2, 0.1 to 0.3, or 0.2 to 0.3. In some embodiments, the molar ratio of soluble acid catalyst (e.g., citric acid catalyst) to total dextrose monomer, galactose monomer, and mannose monomer content is in a range of 0.050 to 0.052. In some embodiments, the molar ratio of soluble acid catalyst (e.g., citric acid catalyst) to total dextrose monomer, galactose monomer, and mannose monomer content is in a range of 0.020 to 0.035. In some embodiments, the molar ratio of soluble acid catalyst (e.g., citric acid catalyst) to total dextrose monomer, galactose monomer, and mannose monomer content is 0.028.

In some embodiments, water is added to the reaction mixture to quench the reaction by bringing the temperature of the reaction mixture to 100° C. or below. In some embodiments, the water used for quenching is deionized water. In some embodiments, the water used for quenching is USP water. In some embodiments, the water has a temperature of about 60° C. to about 100° C. In certain embodiments, the water used for quenching is about 95° C. In some embodiments, the water is added to the reaction mixture under conditions sufficient to avoid solidifying the mixture.

The viscosity of the reaction mixture may be measured and/or altered over the course of the reaction. In general, viscosity refers to a measurement of a fluid's internal resistance to flow (e.g., “thickness”) and is expressed in centipoise (cP) or pascal-seconds. In some embodiments, the viscosity of the reaction mixture is between about 100 cP and about 95,000 cP, about 5,000 cP and about 75,000 cP, about 5,000 and about 50,000 cP, or about 10,000 and about 50,000 cP. In certain embodiments, the viscosity of the reaction mixture is between about 50 cP and about 200 cP.

In some embodiments, oligosaccharide compositions provided herein may be subjected to one or more additional processing steps. Additional processing steps may include, for example, purification steps. Purification steps may include, for example, separation, demonomerization, dilution, concentration, filtration, desalting or ion-exchange, chromatographic separation, or decolorization, or any combination thereof.

In certain embodiments, the methods described herein further include a dilution step. In some embodiments, deionized water is used for dilution. In certain embodiments, USP water is used for dilution. In certain embodiments, after dilution, the oligosaccharide composition comprises water in a range of about 5-75, 25-65, 35-65, 45-55, or 47-53 weight percent. In certain embodiments, after dilution, the oligosaccharide composition comprises water in a range of about 45-55 weight percent.

In some embodiments, the methods described herein further include a decolorization step. The one or more oligosaccharide compositions produced may undergo a decolorization step using appropriate methods, including, for example, treatment with an absorbent, activated carbon, chromatography (e.g., using ion exchange resin), and/or filtration (e.g., microfiltration).

In some embodiments, the one or more oligosaccharide compositions produced are contacted with a material to remove salts, minerals, and/or other ionic species. For example, in certain embodiments, the one or more oligosaccharide compositions produced are flowed through an anionic exchange column. In other embodiments, oligosaccharide compositions produced are flowed through an anionic/cationic exchange column pair.

In some embodiments, the methods described herein may further include a concentration step. For example, in some embodiments, the oligosaccharide compositions may be subjected to evaporation (e.g., vacuum evaporation) to produce a concentrated oligosaccharide composition. In other embodiments, the oligosaccharide compositions may be subjected to a spray drying step to produce an oligosaccharide powder. In certain embodiments, the oligosaccharide compositions may be subjected to both an evaporation step and a spray drying step. In some embodiments, the oligosaccharide compositions be subjected to a lyophilization (e.g., freeze drying) step to remove water and produce powdered product.

In some embodiments, the methods described herein further include a fractionation step. Oligosaccharide compositions prepared and purified may be subsequently separated by molecular weight using any method known in the art, including, for example, high-performance liquid chromatography, adsorption/desorption (e.g. low-pressure activated carbon chromatography), or filtration (for example, ultrafiltration or diafiltration). In certain embodiments, oligosaccharide compositions are separated into pools representing 60%, 65%, 70%, 75%, 80%, 85%, 90%, 95%, 97%, 98%, or greater than 98% short (about DP1-2), medium (about DP3-10), long (about DP11-18), or very long (about DP>18) species.

In certain embodiments, prepared oligosaccharide compositions are fractionated by adsorption onto a carbonaceous material and subsequent desorption of fractions by washing the material with mixtures of an organic solvent in water at a concentration of 1%, 5%, 10%, 20%, 50%, or 100%. In one embodiment, the adsorption material is activated charcoal. In another embodiment, the adsorption material is a mixture of activated charcoal and a bulking agent such as diatomaceous earth or Celite 545 in 5%, 10%, 20%, 30%, 40%, or 50% portion by volume or weight.

In further embodiments, prepared oligosaccharide compositions are separated by passage through a high-performance liquid chromatography system. In certain variations, prepared oligosaccharide compositions are separated by ion-affinity chromatography, hydrophilic interaction chromatography, or size-exclusion chromatography including gel-permeation and gel-filtration.

In some embodiments, catalyst is removed by filtration. In certain embodiments, a 0.45 μm filter is used to remove catalyst during filtration. In other embodiments, low molecular weight materials are removed by filtration methods. In certain variations, low molecular weight materials may be removed by dialysis, ultrafiltration, diafiltration, or tangential flow filtration. In certain embodiments, the filtration is performed in static dialysis tube apparatus. In other embodiments, the filtration is performed in a dynamic flow filtration system. In other embodiments, the filtration is performed in centrifugal force-driven filtration cartridges. In certain embodiments, the reaction mixture is cooled to below about 85° C. before filtration.

In certain embodiments, the mean degree of polymerization of all oligosaccharides is in a range of 6-16. In certain embodiments, the mean degree of polymerization of all oligosaccharides is in a range of 10-15. In some embodiments, the mean degree of polymerization of all oligosaccharides is in a range of 7-15, 7-12, 7-10, 7-8, 9-10, 10-11, 11-12, 11-15, 12-13, 12-14 13-14, 14-15, 15-16, 17-18, 15-20, 3-8, 4-7, or 5-6.

In certain embodiments, the weight percent of dextrose monomer, galactose monomer, and mannose monomer in the oligosaccharide composition is in a range of 10-18. In certain embodiments, the weight percent of dextrose monomer, galactose monomer, and mannose monomer in the oligosaccharide composition is in a range of 11-17. In certain embodiments, the weight percent of dextrose monomer, galactose monomer, and mannose monomer in the oligosaccharide composition is in a range of 12-16. In certain embodiments, the weight percent of dextrose monomer, galactose monomer, and mannose monomer in the oligosaccharide composition is in a range of 13-15.

In some embodiments, the oligosaccharide composition is a mixture of polymers of dextrose, galactose, and mannose in proportions of approximately 45%, 45%, and 10%, by weight respectively. The formula is H—[C₆H₉₋₁₁O₅]ₙ—OH, where the total number of monomer units in a single polymer of the mixture ranges from 2 to approximately 60 (n=2-60), with a mean value for the mixture of approximately 12.6 monomer units. Each monomer unit may be unsubstituted, singly, doubly, or triply substituted with another dextrose, galactose, or mannose unit by any glycosidic isomer.

In some embodiments, the oligosaccharide composition comprises water in a range of 5-75 weight percent. In some embodiments, the oligosaccharide composition comprises water in a range of 25-65 weight percent. In some embodiments, the oligosaccharide composition comprises water in a range of 35-65 weight percent. In some embodiments, the oligosaccharide composition comprises water in a range of 45-55 weight percent.

In some embodiments, the oligosaccharide composition comprises oligosaccharides that have a MWw (g/mol) in a range of 1905-2290. In some embodiments, the oligosaccharide composition comprises oligosaccharides that have a MWw (g/mol) in a range of 1753-2395. In some embodiments, the oligosaccharide composition comprises oligosaccharides that have a MWw (g/mol) in a range of 1750-2400. In some embodiments, the oligosaccharide composition comprises oligosaccharides that have a MWw (g/mol) in a range of 1500-2500. In some embodiments, the oligosaccharide composition comprises oligosaccharides that have a MWw (g/mol) in a range of 1800-2000. In some embodiments, the oligosaccharide composition comprises oligosaccharides that have a MWw (g/mol) in a range of 2000-2300. In some embodiments, the oligosaccharide composition comprises oligosaccharides that have a MWw (g/mol) in a range of 1515-2630. In some embodiments, the oligosaccharide composition comprises oligosaccharides that have a MWw (g/mol) in a range of 1500-2500. In some embodiments, the oligosaccharide composition comprises oligosaccharides that have a MWw (g/mol) in a range of 1740-2400. In some embodiments, the oligosaccharide composition comprises oligosaccharides that have a MWw (g/mol) in a range of 1700-2300. In some embodiments, the oligosaccharide composition comprises oligosaccharides that have a MWw (g/mol) in a range of 1800-1900, 1900-2000, 2000-2100, 2100-2200, 2200-2300, 2300-2400, or 2400-2500.

In some embodiments, the oligosaccharide composition comprises oligosaccharides that have a MWn (g/mol) in a range of 1030-1095. In some embodiments, the oligosaccharide composition comprises oligosaccharides that have a MWn (g/mol) in a range of 981-1214. In some embodiments, the oligosaccharide composition comprises oligosaccharides that have a MWn (g/mol) in a range of 980-1220 In some embodiments, the oligosaccharide composition comprises oligosaccharides that have a MWn (g/mol) in a range of 1000-1050. In some embodiments, the oligosaccharide composition comprises oligosaccharides that have a MWn (g/mol) in a range of 1050-1100. In some embodiments, the oligosaccharide composition comprises oligosaccharides that have a MWn (g/mol) in a range of 890-1300. In some embodiments, the oligosaccharide composition comprises oligosaccharides that have a MWn (g/mol) in a range of 975-1155. In some embodiments, the oligosaccharide composition comprises oligosaccharides that have a MWn (g/mol) in a range of 875-1180. In some embodiments, the oligosaccharide composition comprises oligosaccharides that have a MWn (g/mol) in a range of 940-1120. In some embodiments, the oligosaccharide composition comprises oligosaccharides that have a MWn (g/mol) in a range of 900-950, 950-1000, 1000-1050, 1050-1100, 1100-1150, 1150-1200, or 1200-1250.

In some embodiments, a solution comprising the oligosaccharide composition has a pH in a range of 1.50-6.00. In some embodiments, a solution comprising the oligosaccharide composition has a pH in a range of 1.50-5.00. In some embodiments, a solution comprising the oligosaccharide composition has a pH in a range of 2.00-4.00. In some embodiments, a solution comprising the oligosaccharide composition has a pH in a range of 2.50-3.50.

In some embodiments, the oligosaccharide composition comprises oligosaccharides that have a degree of branching in a range of about 8.5% to about 32%. In some embodiments, the oligosaccharide composition comprises oligosaccharides that have a degree of branching in a range of about 10% to about 35%. In some embodiments, the oligosaccharide composition comprises oligosaccharides that have a degree of branching in a range of about 13% to about 29%. In some embodiments, the oligosaccharide composition comprises oligosaccharides that have a degree of branching in a range of 5-50%, 5-40%, 5-30%, 5-20%, 5-15%, 10-50%, 10-40%, 10-30%, 10-25%, 15-30%, or 15-20%.

In some embodiments, the oligosaccharide composition comprises oligomers having two or more repeat units (DP2+) in a range of 80-100 weight percent. In some embodiments, the oligosaccharide composition comprises oligomers having two or more repeat units (DP2+) in a range of 86-96 weight percent. In some embodiments, the oligosaccharide composition comprises oligomers having two or more repeat units (DP2+) in a range of 86-91 weight percent. In some embodiments, the oligosaccharide composition comprises oligomers having two or more repeat units (DP2+) in a range of 91-96 weight percent. In some embodiments, the oligosaccharide composition comprises oligomers having two or more repeat units (DP2+) in a range of 81-100 weight percent. In some embodiments, the oligosaccharide composition comprises oligomers having two or more repeat units (DP2+) in a range of 80-94 weight percent. In some embodiments, the oligosaccharide composition comprises oligomers having two or more repeat units (DP2+) in a range of 91-96 weight percent. In some embodiments, the oligosaccharide composition comprises oligomers having two or more repeat units (DP2+) in a range of 80-85, 85-87, 86-88, 87-90, 88-91, 89-92, 90-93, 91-94, 92-95, 93-96, or 95-98 weight percent.

In some embodiments, the oligosaccharide composition has a polydispersity index (PDI) of 1.8-2.0. In some embodiments, the oligosaccharide composition has a polydispersity index (PDI) of 1.8-2.1. In some embodiments, the oligosaccharide composition has a PDI of 1.0-1.2, 1.2-1.3, 1.3-1.4, 1.4-1.5, 1.5-1.6, 1.7-1.8, 1.8-2.0, 2.0-2.2, 2.2-2.4, or 2.4-2.6. In some embodiments, the oligosaccharide composition has a PDI of about 1.6, about 1.7, about 1.8, about 1.9, about 2.0, about 2.1, or about 2.2.

In some embodiments, the MWw, MWn, PDI, monomer content (DP1) and/or DP2+ values of oligosaccharides in an oligosaccharide composition are determined using the size exclusion chromatography method described in Example 10.

In some embodiments, the degree of polymerization (DP1-DP7) of oligosaccharides in an oligosaccharide composition are determined using the size exclusion chromatography method described in Example 12.

In some embodiments, the oligosaccharide composition comprises oligomers having at least three linked monomer units (DP3+) in a range of 80-95 weight percent. In some embodiments, the oligosaccharide composition comprises oligomers having at least three linked monomer units (DP3+) in a range of 85-90 weight percent. In some embodiments, the oligosaccharide composition comprises oligomers having at least three linked monomer units (DP3+) in a range of 80-85, 85-87, 86-88, 87-90, 88-91, 89-92, 90-93, 91-94, or 92-95 weight percent.

In some embodiments, the oligosaccharide composition comprises 4.20% to 6.28% monomer (DP1). In some embodiments, the oligosaccharide composition comprises 4% to 5%, 5% to 6%, or 6% to 7% monomer (DP1). In some embodiments, the oligosaccharide composition comprises 6.20% to 8.83% disaccharide (DP2). In some embodiments, the oligosaccharide composition comprises 6% to 6.5%, 6.5% to 7%, 7.5% to 8%, 8% to 8.5%, or 8.5% to 9% disaccharide (DP2). In some embodiments, the oligosaccharide composition comprises 84.91% to 89.58% oligomers having at least three linked monomer units (DP3+). In some embodiments, the oligosaccharide composition comprises 84% to 85%, 85% to 86%, 86% to 87%, 87% to 88%, or 88% to 90% oligomers having at least three linked monomer units (DP3+).

In some embodiments, the oligosaccharide composition comprises less than 0.10% total impurities (excluding monomer). In some embodiments, the oligosaccharide composition comprises less than 0.05% total impurities (excluding monomer). In some embodiments, the oligosaccharide composition comprises less than 0.20%, 0.15%, 0.10%, or 0.05% total impurities (excluding monomer). In some embodiments, the oligosaccharide composition comprises less than 0.10% w/w levoglucosan, less than 0.10% w/w glucuronic acid, less than 0.10% w/w lactic acid, less than 0.10% w/w formic acid, less than 0.10% w/w acetic acid, and less than 0.10% w/w HMF. In some embodiments, the oligosaccharide composition comprises 0.35% w/w levoglucosan, 0.03% w/w lactic acid, and/or 0.06% w/w formic acid. In some embodiments, the oligosaccharide composition comprises 0.28-0.43% w/w levoglucosan, 0.00-0.03% w/w lactic acid, and/or 0.05-0.07% w/w formic acid.

In some embodiments, the oligosaccharide composition comprises a MWw of 1753-2395, a MWn of 981-1214, and/or a PDI of 1.8-2.0.

The oligosaccharide compositions described herein, and prepared according to the methods described herein, can be characterized and distinguished from prior art compositions using permethylation analysis. Sec, e.g., Zhao, Y., et al. ‘Rapid, sensitive structure analysis of oligosaccharides,’ PNAS Mar. 4, 1997 94 (5) 1629-1633; Kailemia, M. J., et al. ‘Oligosaccharide analysis by mass spectrometry: A review of recent developments,’ Anal Chem. 2014 Jan. 7; 86 (1): 196-212. Accordingly, in another aspect, oligosaccharide compositions are provided herein that comprise a plurality of oligosaccharides that are minimally digestible in humans, the plurality of oligosaccharides comprising monomer radicals. The molar percentages of different types of monomer radicals in the plurality of oligosaccharides can be quantified using a permethylation assay. The permethylation assay is performed on a de-monomerized sample of the composition.

In some embodiments, the plurality of oligosaccharides comprises two or more monomer radicals selected from radicals (1)-(40):
(1) t-manopyranose monoradicals, representing 3.0-4.1 mol % of monomer radicals in the plurality of oligosaccharides;
(2) t-glucopyranose monoradicals, representing 11.4-16.3 mol % of monomer radicals in the plurality of oligosaccharides;
(3) t-galactofuranose monoradicals, representing 1.3-7.8 mol % of monomer radicals in the plurality of oligosaccharides;
(4) t-glucofuranose monoradicals, representing 0-1.4 mol % of monomer radicals in the plurality of oligosaccharides;
(5) t-galactopyranose monoradicals, representing 8.3-12.5 mol % of monomer radicals in the plurality of oligosaccharides;
(6) 3-glucopyranose monoradicals, representing 3.0-4.9 mol % of monomer radicals in the plurality of oligosaccharides;
(7) 2-manopyranose and/or 3-manopyranose monoradicals, representing 1.2-1.9 mol % of monomer radicals in the plurality of oligosaccharides;
(8) 2-glucopyranose monoradicals, representing 2.4-3.2 mol % of monomer radicals in the plurality of oligosaccharides;
(9) 2-galactofuranose and/or 2-glucofuranose monoradicals, representing 0.9-2.3 mol % of monomer radicals in the plurality of oligosaccharides;
(10) 3-galactopyranose monoradicals, representing 2.9-3.9 mol % of monomer radicals in the plurality of oligosaccharides;
(11) 4-manopyranose and/or 5-manofuranose and/or 3-galactofuranose monoradicals, representing 1.7-2.9 mol % of monomer radicals in the plurality of oligosaccharides;
(12) 6-manopyranose monoradicals, representing 2.0-2.9 mol % of monomer radicals in the plurality of oligosaccharides;
(13) 2-galactopyranose monoradicals, representing 1.8-2.7 mol % of monomer radicals in the plurality of oligosaccharides;
(14) 6-glucopyranose monoradicals, representing 7.6-10.8 mol % of monomer radicals in the plurality of oligosaccharides;
(15) 4-galactopyranose and/or 5-galactofuranose monoradicals, representing 2.6-3.8 mol % of monomer radicals in the plurality of oligosaccharides;
(16) 4-glucopyranose and/or 5-glucofuranose and/or 6-manofuranose monoradicals, representing 3.0-4.5 mol % of monomer radicals in the plurality of oligosaccharides;
(17) 6-glucofuranose monoradicals, representing 0-1.6 mol % of monomer radicals in the plurality of oligosaccharides;
(18) 6-galactofuranose monoradicals, representing 1.4-5.0 mol % of monomer radicals in the plurality of oligosaccharides;
(19) 6-galactopyranose monoradicals, representing 5.8-9.1 mol % of monomer radicals in the plurality of oligosaccharides;
(20) 3,4-galactopyranose and/or 3,5-galactofuranose and/or/2,3-galactopyranose diradicals, representing 0.9-1.4 mol % of monomer radicals in the plurality of oligosaccharides;
(21) 3,4-glucopyranose and/or 3,5-glucofuranose diradicals, representing 0-1.1 mol % of monomer radicals in the plurality of oligosaccharides;
(22) 2,4-glucopyranose and/or 2,5-glucofuranose and/or 2,4-galactopyranose and/or 2,5-galactofuranose diradicals, representing 0.9-1.4 mol % of monomer radicals in the plurality of oligosaccharides;
(23) 4,6-manopyranose and/or 5,6-manofuranose diradicals, representing 0.5-0.7 mol % of monomer radicals in the plurality of oligosaccharides;
(24) 3,6-manofuranose diradicals, representing 0-0.1 mol % of monomer radicals in the plurality of oligosaccharides;
(25) 3,6-glucopyranose diradicals, representing 1.4-2.8 mol % of monomer radicals in the plurality of oligosaccharides;
(26) 3,6-manopyranose and/or 2,6-manofuranose diradicals, representing 0.4-0.7 mol % of monomer radicals in the plurality of oligosaccharides;
(27) 2,6-manopyranose diradicals, representing 0.3-0.5 mol % of monomer radicals in the plurality of oligosaccharides;
(28) 3,6-glucofuranose diradicals, representing 0.1-0.4 mol % of monomer radicals in the plurality of oligosaccharides;
(29) 2,6-glucopyranose and/or 4,6-glucopyranose and/or 5,6-glucofuranose diradicals, representing 1.1-3.6 mol % of monomer radicals in the plurality of oligosaccharides;
(30) 3,6-galactofuranose diradicals, representing 0.9-1.4 mol % of monomer radicals in the plurality of oligosaccharides;
(31) 4,6-galactopyranose and/or 5,6-galactofuranose diradicals, representing 2.1-2.9 mol % of monomer radicals in the plurality of oligosaccharides;
(32) 3,6-galactopyranose and/or 2,6-galactofuranose diradicals, representing 1.6-3.0 mol % of monomer radicals in the plurality of oligosaccharides;
(33) 2,6-galactopyranose diradicals, representing 0.7-1.6 mol % of monomer radicals in the plurality of oligosaccharides;
(34) 3,4,6-manopyranose and/or 3,5,6-manofuranose and/or 2,3,6-manofuranose triradicals, representing 0-0.3 mol % of monomer radicals in the plurality of oligosaccharides;
(35) 3,4,6-galactopyranose and/or 3,5,6-galactofuranose and/or 2,3,6-galactofuranose triradicals, representing 0.5-1.1 mol % of monomer radicals in the plurality of oligosaccharides;
(36) 3,4,6-glucopyranose and/or 3,5,6-glucofuranose triradicals, representing 0.2-0.5 mol % of monomer radicals in the plurality of oligosaccharides;
(37) 2,3,6-manopyranose and/or 2,4,6-manopyranose and/or 2,5,6-manofuranose triradicals, representing 0-0.5 mol % of monomer radicals in the plurality of oligosaccharides;
(38) 2,4,6-glucopyranose and/or 2,5,6-glucofuranose triradicals, representing 0-1.4 mol % of monomer radicals in the plurality of oligosaccharides;
(39) 2,3,6-galactopyranose and/or 2,4,6-galactopyranose and/or 2,5,6-galactofuranose triradicals, representing 0.4-0.9 mol % of monomer radicals in the plurality of oligosaccharides; and
(40) 2,3,6-glucopyranose triradicals, representing 0.1-0.5 mol % of monomer radicals in the plurality of oligosaccharides.

In some embodiments, about 8-30% of the total glycosidic bonds in an oligosaccharide composition are 1,2 glycosidic bonds. In some embodiments, about 10.5-25% of the total glycosidic bonds in an oligosaccharide composition are 1,2 glycosidic bonds. In some embodiments, about 9.5-32% of the total glycosidic bonds in an oligosaccharide composition are 1.2 glycosidic bonds. In some embodiments, about 13-27% of the total glycosidic bonds in an oligosaccharide composition are 1.2 glycosidic bonds. In some embodiments, 5-50%, 5-40%, 5-30%, 5-20%, 5-15%, 10-50%, 10-40%, 10-30%, 10-25%, 15-30%, or 15-20% of the total glycosidic bonds in an oligosaccharide composition are 1,2 glycosidic bonds. In some embodiments, about 15% or about 16% of the total glycosidic bonds in an oligosaccharide composition are 1,2 glycosidic bonds.

In some embodiments, about 14.5-34% of the total glycosidic bonds in an oligosaccharide composition are 1.3 glycosidic bonds. In some embodiments, about 17-30% of the total glycosidic bonds in an oligosaccharide composition are 1,3 glycosidic bonds. In some embodiments, about 9.5-27% of the total glycosidic bonds in an oligosaccharide composition are 1,3 glycosidic bonds. In some embodiments, about 12.5-23.5% of the total glycosidic bonds in an oligosaccharide composition are 1,3 glycosidic bonds. In some embodiments, 5-50%, 10-40%, 10-30%, 10-20%, 5-15%, 10-50%, 10-40%, 10-30%, 10-25%, 15-30%, or 15-20% of the total glycosidic bonds in an oligosaccharide composition are 1,3 glycosidic bonds. In some embodiments, about 19% or about 20% of the total glycosidic bonds in an oligosaccharide composition are 1,3 glycosidic bonds.

In some embodiments, about 10-26% of the total glycosidic bonds in an oligosaccharide composition are 1,4 glycosidic bonds. In some embodiments, about 12-22% of the total glycosidic bonds in an oligosaccharide composition are 1,4 glycosidic bonds. In some embodiments, about 10-29.5% of the total glycosidic bonds in an oligosaccharide composition are 1,4 glycosidic bonds. In some embodiments, about 13-25% of the total glycosidic bonds in an oligosaccharide composition are 1,4 glycosidic bonds. In some embodiments, 5-50%, 10-40%, 10-30%, 10-20%, 5-15%, 10-50%, 10-40%, 10-30%, 10-25%, 15-30%, or 15-20% of the total glycosidic bonds in an oligosaccharide composition are 1,4 glycosidic bonds. In some embodiments, about 27% of the total glycosidic bonds in an oligosaccharide composition are 1,4 glycosidic bonds.

In some embodiments, about 32-57% of the total glycosidic bonds in an oligosaccharide composition are 1,6 glycosidic bonds. In some embodiments, about 35-52% of the total glycosidic bonds in an oligosaccharide composition are 1,6 glycosidic bonds. In some embodiments, about 23-65% of the total glycosidic bonds in an oligosaccharide composition are 1,6 glycosidic bonds. In some embodiments, about 30-56% of the total glycosidic bonds in an oligosaccharide composition are 1,6 glycosidic bonds. In some embodiments, 15-70%, 20-60%, 20-40%, 25-50%, 30-50%, 30-40%, or 30-60% of the total glycosidic bonds in an oligosaccharide composition are 1,6 glycosidic bonds. In some embodiments, about 44% of the total glycosidic bonds in an oligosaccharide composition are 1,2 glycosidic bonds.

In some embodiments, an oligosaccharide composition comprises 17.5-43% total furanose. In some embodiments, an oligosaccharide composition comprises 20.5-37% total furanose. In some embodiments, an oligosaccharide composition comprises 14-60% total furanose. In some embodiments, an oligosaccharide composition comprises 20.5-50% total furanose. In some embodiments, an oligosaccharide composition comprises 10-60%, 10-50%, 15-40%, 20-40%, 20-30%, or 30-50% total furanose. In some embodiments, an oligosaccharide composition comprises about 35% or about 36% total furanose.

In some embodiments, the oligosaccharide composition comprises at least one glucofuranose or glucopyranose radical, at least one manofuranose or manopyranose radical, and at least one galactofuranose or galactopyranose radical.

In some embodiments, an oligosaccharide composition is provided, comprising a plurality of oligosaccharides comprising monomer radicals (1)-(40) in the molar percentages shown in Table 2.

TABLE 2
Permethylation Data

| | Mean mol % +3 | Mean | Mean mol % −3 |
|---|---|---|---|
| Radicals | STD | mol % | STD |
| t-manopyranose | 4.10% | 3.56% | 3.02% |
| t-glucopyranose | 16.33% | 13.89% | 11.44% |
| t-galactofuranose | 7.78% | 4.52% | 1.26% |
| t-glucofuranose | 1.38% | 0.64% | 0.00% |
| t-galactopyranose | 12.48% | 10.38% | 8.29% |
| 3-glucopyranose | 4.88% | 3.95% | 3.02% |
| 2-manopyranose and/or | 1.94% | 1.57% | 1.20% |
| 3-manopyranose | | | |
| 2-glucopyranose | 3.22% | 2.83% | 2.44% |
| 2-galactofuranose and/or | 2.32% | 1.62% | 0.93% |
| 2-glucofuranose | | | |
| 3-galactopyranose | 3.92% | 3.43% | 2.94% |
| 4-manopyranose and/or | 2.93% | 2.34% | 1.75% |
| 5-manofuranose and/or | | | |
| 3-galactofuranose | | | |
| 6-manopyranose | 2.87% | 2.44% | 2.01% |
| 2-galactopyranose | 2.71% | 2.28% | 1.85% |
| 6-glucopyranose | 10.78% | 9.22% | 7.66% |
| 4-galactopyranose and/or | 3.80% | 3.22% | 2.65% |
| 5-galactofuranose | | | |
| 4-glucopyranose and/or | 4.25% | 3.66% | 3.06% |
| 5-glucofuranose and/or | | | |
| 6-manofuranose | | | |
| 6-glucofuranose | 1.55% | 0.81% | 0.08% |
| 6-galactofuranose | 4.96% | 3.19% | 1.42% |
| 6-galactopyranose | 9.06% | 7.44% | 5.81% |
| 3,4-galactopyranose and/or | 1.42% | 1.16% | 0.90% |
| 3,5-galactofuranose and/or | | | |
| 2,3-galactopyranose | | | |
| 3,4-glucopyranose and/or | 1.04% | 0.43% | 0.00% |
| 3,5-glucofuranose | | | |
| 2,4-glucopyranose and/or | 1.39% | 1.16% | 0.92% |
| 2,5-glucofuranose and/or | | | |
| 2,4-galactopyranose and/or | | | |
| 2,5-galactofuranose | | | |
| 4,6-manopyranose and/or | 0.69% | 0.59% | 0.49% |
| 5,6-manofuranose | | | |
| 3,6-manofuranose | 0.11% | 0.02% | 0.00% |
| 3,6-glucopyranose | 2.80% | 2.10% | 1.40% |
| 3,6-manopyranose and/or | 0.67% | 0.53% | 0.39% |
| 2,6-manofuranose | | | |
| 2,6-manopyranose | 0.54% | 0.41% | 0.28% |
| 3,6-glucofuranose | 0.39% | 0.27% | 0.16% |
| 2,6-glucopyranose and/or | 3.58% | 2.33% | 1.08% |
| 4,6-glucopyranose and/or | | | |
| 5,6-glucofuranose | | | |
| 3,6-galactofuranose | 1.37% | 1.15% | 0.93% |
| 4,6-galactopyranose and/or | 2.86% | 2.48% | 2.11% |
| 5,6-galactofuranose | | | |
| 3,6-galactopyranose and/or | 2.98% | 2.28% | 1.58% |
| 2,6-galactofuranose | | | |
| 2,6-galactopyranose | 1.62% | 1.15% | 0.68% |
| 3,4,6-manopyranose and/or | 0.30% | 0.07% | 0.00% |
| 3,5,6-manofuranose and/or | | | |
| 2,3,6-manofuranose | | | |
| 3,4,6-galactopyranose and/or | 1.11% | 0.82% | 0.53% |
| 3,5,6-galactofuranose and/or | | | |
| 2,3,6-galactofuranose | | | |
| 3,4,6-glucopyranose and/or | 0.47% | 0.35% | 0.22% |
| 3,5,6-glucofuranose | | | |
| 2,3,6-manopyranose and/or | 0.49% | 0.17% | 0.00% |
| 2,4,6-manopyranose and/or | | | |
| 2,5,6-manofuranose | | | |
| 2,4,6-glucopyranose and/or | 1.36% | 0.56% | 0.00% |
| 2,5,6-glucofuranose | | | |
| 2,3,6-galactopyranose and/or | 0.91% | 0.66% | 0.41% |
| 2,4,6-galactopyranose and/or | | | |
| 2,5,6-galactofuranose | | | |
| 2,3,6-glucopyranose | 0.48% | 0.31% | 0.13% |

In certain embodiments, the oligosaccharide compositions are free from monomer. In other embodiments, the oligosaccharide compositions comprise monomer.

The oligosaccharide compositions described herein, and prepared according to the methods described herein, can be characterized and distinguished from prior art compositions using two-dimensional heteronuclear NMR. Accordingly, in another aspect, oligosaccharide compositions are provided that comprise a plurality of oligosaccharides that are minimally digestible in humans, the compositions being characterized by a heteronuclear single quantum correlation (HSQC) NMR spectrum comprising signals 5, 6, 7, and 15, each signal having a center position and an area:

| | Center Position | | |
|---|---|---|---|
| | (ppm) | | Area under the curve (AUC) |
| Signal | ¹H | ¹³C | (% of total areas of all signals) |
| 5 | 3.96 | 70.62 | 9.28-10.71 |
| 6 | 3.92 | 71.26 | 1.52-2.03 |
| 7 | 3.55 | 71.34 | 3.40-6.13 |
| 15 | 4.44 | 103.86 | 1.84-2.44 |

In some embodiments, the spectrum further comprises 1-2 (e.g., one or two) signals selected from signals 10 and 14, and defined as follows:

| | Center Position | | |
|---|---|---|---|
| | (ppm) | | Area under the curve (AUC) |
| Signal | ¹H | ¹³C | (% of total areas of all signals) |
| 10 | 3.33 | 73.74 | 10.21-12.09 |
| 14 | 4.5 | 103.29 | 5.03-6.41 |

In some embodiments, the spectrum further comprises 1-3 (e.g., one, two, or three) signals selected from signals 11, 12, and 13, and defined as follows:

| | Center Position | | |
|---|---|---|---|
| | (ppm) | | Area under the curve (AUC) |
| Signal | ¹H | ¹³C | (% of total areas of all signals) |
| 11 | 4.06 | 77.34 | 3.68-4.50 |
| 12 | 4.11 | 81.59 | 3.10-3.82 |
| 13 | 4.96 | 98.7 | 10.65-12.31 |

In some embodiments, the spectrum comprises 1-3 (e.g., one, two, or three) signals selected from signals 11, 12, and 13, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

In some embodiments, the spectrum comprises 1-15 (e.g., one, two, or three) signals selected from signals 1-15, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 18.59-27.53 |
| 2 | 3.75 | 66.06 | 2.79-7.27 |
| 3 | 3.97 | 66.15 | 1.82-3.8 |
| 4 | 3.96 | 69.28 | 0.71-4.47 |
| 5 | 3.96 | 70.62 | 8.81-11.19 |
| 6 | 3.92 | 71.26 | 1.35-2.2 |
| 7 | 3.55 | 71.34 | 2.48-7.04 |
| 8 | 3.97 | 71.56 | 3.06-4.74 |
| 9 | 3.72 | 72.35 | 4.16-11.64 |
| 10 | 3.33 | 73.74 | 9.58-12.72 |
| 11 | 4.06 | 77.34 | 3.4-4.78 |
| 12 | 4.11 | 81.59 | 2.86-4.06 |
| 13 | 4.96 | 98.7 | 10.09-12.87 |
| 14 | 4.5 | 103.29 | 4.57-6.87 |
| 15 | 4.44 | 103.86 | 1.64-2.64 |

In some embodiments, the spectrum comprises 1-15 (e.g., one, two, or three) signals selected from signals 1-15, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.18-27.11 |
| 2 | 3.75 | 66.06 | 3.31-6.1 |
| 3 | 3.97 | 66.15 | 2.37-3.69 |
| 4 | 3.96 | 69.28 | 0.42-4.72 |
| 5 | 3.96 | 70.62 | 8.43-11.69 |
| 6 | 3.92 | 71.26 | 1.09-3.1 |
| 7 | 3.55 | 71.34 | 4.01-6.37 |
| 8 | 3.97 | 71.56 | 2.77-4.29 |
| 9 | 3.72 | 72.35 | 6.28-9.25 |
| 10 | 3.33 | 73.74 | 10.48-12 |
| 11 | 4.06 | 77.34 | 3.04-4.22 |
| 12 | 4.11 | 81.59 | 2.63-3.57 |
| 13 | 4.96 | 98.7 | 9.9-13.39 |
| 14 | 4.5 | 103.29 | 4.45-6.7 |
| 15 | 4.44 | 103.86 | 1.6-2.63 |

In some embodiments, the spectrum comprises 1-15 (e.g., one, two, or three) signals selected from signals 1-15, and defined as follows:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

In some embodiments, signals 5, 6, 7, 15, 10, 14, 11, 12, and 13 are each further characterized by an ¹H integral region and a ¹³C integral region, defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 15 | 4.44 | 4.41 | 4.46 | 103.86 | 103.56 | 104.15 |
| 10 | 3.33 | 3.27 | 3.4 | 73.74 | 73.26 | 74.22 |
| 14 | 4.5 | 4.47 | 4.54 | 103.29 | 102.87 | 103.70 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |

In some embodiments, signals 1-15 are each characterized by an ¹H integral region and a ¹³C integral region, defined as follows:

In some embodiments, oligosaccharide compositions having a plurality of oligosaccharides that are being characterized by a multiplicity-edited gradient-enhanced ¹H-¹³C heteronuclear single quantum correlation (HSQC) NMR spectrum comprises one or more of signals 1-15 having an ¹H integral region and a ¹³C integral region, defined as follows in Table 7:

TABLE 7
Coordinates of HSQC NMR integral regions

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 15 | 4.44 | 4.41 | 4.46 | 103.86 | 103.56 | 104.15 |
| 10 | 3.33 | 3.27 | 3.4 | 73.74 | 73.26 | 74.22 |
| 14 | 4.5 | 4.47 | 4.54 | 103.29 | 102.87 | 103.70 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |

As used herein, the term “heteronuclear single quantum correlation (HSQC) NMR” can be used interchangeably with the term “heteronuclear single quantum coherence (HSQC) NMR.”

As used herein, the term “area under the curve” or “AUC” refers to the relative size (i.e., relative intensity, relative volume) of peaks/signals in an NMR spectrum (e.g., relative size of signals 1-15 of an HSQC NMR spectrum of the selected oligosaccharide composition). The AUC or relative size of peaks/signals defined herein represents the integration of integral regions using an elliptical shape. The elliptical shape can be defined by major axis coordinates and minor axis coordinates (e.g., major axis coordinates (F2 dimension; 1H) and minor axis coordinates (F1 dimension; 13C)). Thus, an AUC can then be determined by integrating within the confines of that elliptical shape to obtain the volume above or below the ellipse.

In some embodiments, oligosaccharide compositions are provided that are characterized by a HSQC NMR spectrum comprising a peak corresponding to the presence of one or more moieties, wherein each moiety comprises one or more HSQC NMR detectable bonds and/or atoms. In some embodiments, oligosaccharide compositions are provided that are characterized by a HSQC NMR spectrum comprising a peak corresponding to the presence of one or more moieties as described in Example 9. In some embodiments, oligosaccharide compositions are provided that are characterized by a HSQC NMR spectrum comprising a peak corresponding to a Galp-a-reducing moiety. In some embodiments, oligosaccharide compositions are provided that are characterized by a HSQC NMR spectrum comprising a peak corresponding to a Glup-a-reducing moiety. In some embodiments, oligosaccharide compositions are provided that are characterized by a HSQC NMR spectrum comprising a peak corresponding to a Manp-a-reducing moiety. In some embodiments, oligosaccharide compositions are provided that are characterized by a HSQC NMR spectrum comprising a peak corresponding to a Manp-b-reducing moiety. In some embodiments, oligosaccharide compositions are provided that are characterized by a HSQC NMR spectrum comprising a peak corresponding to a Glup-a,a(1,1) moiety. In some embodiments, oligosaccharide compositions are provided that are characterized by a HSQC NMR spectrum comprising a peak corresponding to a Glup-b-reducing moiety. In some embodiments, oligosaccharide compositions are provided that are characterized by a HSQC NMR spectrum comprising a peak corresponding to a Galp-a(1,3) moiety. In some embodiments, oligosaccharide compositions are provided that are characterized by a HSQC NMR spectrum comprising a peak corresponding to a Galp-b-reducing moiety. In some embodiments, oligosaccharide compositions are provided that are characterized by a HSQC

NMR spectrum comprising a peak corresponding to a Manp-b-reducing moiety. In some embodiments, oligosaccharide compositions are provided that are characterized by a HSQC NMR spectrum comprising a peak corresponding to a Glup-a(1,2) moiety. In some embodiments, oligosaccharide compositions are provided that are characterized by a HSQC NMR spectrum comprising a peak corresponding to a Glup-a(1,6) moiety. In some embodiments, oligosaccharide compositions are provided that are characterized by a HSQC NMR spectrum comprising a peak corresponding to a Glup-a(1,3) moiety. In some embodiments, oligosaccharide compositions are provided that are characterized by a HSQC NMR spectrum comprising a peak corresponding to a Glup-a(1,4) moiety. In some embodiments, oligosaccharide compositions are provided that are characterized by a HSQC NMR spectrum comprising a peak corresponding to a Galp-a(1,6) moiety. In some embodiments, oligosaccharide compositions are provided that are characterized by a HSQC NMR spectrum comprising a peak corresponding to a Manp-a(1,6) moiety. In some embodiments, oligosaccharide compositions are provided that are characterized by a HSQC NMR spectrum comprising a peak corresponding to a Glup-b(1,3) moiety. In some embodiments, oligosaccharide compositions are provided that are characterized by a HSQC NMR spectrum comprising a peak corresponding to a Glup-b(1,4)+b(1,6) moiety. In some embodiments, oligosaccharide compositions are provided that are characterized by a HSQC NMR spectrum comprising a peak corresponding to a Galp-b (1,6) moiety. In some embodiments, oligosaccharide compositions are provided that are characterized by a HSQC NMR spectrum comprising a peak corresponding to a Glup-a,b(1,1) moiety. In some embodiments, oligosaccharide compositions are provided that are characterized by a HSQC NMR spectrum comprising a peak corresponding to a Glup-b (1,2)+Galp-b(1,3)+b(1,4) moiety. In some embodiments, oligosaccharide compositions are provided that are characterized by a HSQC NMR spectrum comprising a peak corresponding to a Galf-b(1,6) moiety.

In some embodiments, oligosaccharide compositions are provided that are characterized by a HSQC NMR spectrum comprising a plurality of peaks corresponding to 1, 2, 3, 4, 5, 6, 7, 8, 9, 10, 11, 12, 13, 14, 15, 16, 17, 18, 19, 20, or all of: a Galp-a-reducing moiety, a Glup-a-reducing moiety, a Manp-a-reducing moiety, a Manp-b-reducing moiety, a Glup-a,a(1,1) moiety, a Glup-b-reducing moiety, a Galp-a(1,3) moiety, a Galp-b-reducing moiety, a Manp-b-reducing moiety, a Glup-a(1,2) moiety, a Glup-a(1,6) moiety, a Glup-a(1,3) moiety, a Glup-a(1,4) moiety, a Galp-a(1,6) moiety, a Manp-a(1,6) moiety, a Glup-b(1,3) moiety, a Glup-b(1,4)+b(1,6) moiety, a Galp-b(1,6) moiety, a Glup-a,b(1,1) moiety, a Glup-b(1,2)+Galp-b(1,3)+b(1,4) moiety, or a Galf-b(1,6) moiety.

In certain embodiments, the NMR spectrum is obtained by subjecting a sample of the composition to HSQC NMR, wherein the sample is a solution in a deuterated solvent. Suitable deuterated solvents in include deuterated acetonitrile, deuterated acetone, deuterated methanol, D₂O, and mixtures thereof. In a particular embodiment, the deuterated solvent is D₂O. In certain embodiments, the NMR spectrum is obtained using the conditions described in Example 9.

Exemplary oligosaccharide compositions may be prepared according to the procedures described herein.

### III. Methods of Use

As described herein, oligosaccharide compositions may be used to treat an inflammatory lung disease or disorder in subjects (e.g., human subjects). In some embodiments, the inflammatory lung disease or disorder is a chronic disease or disorder. In some embodiments, the inflammatory lung disease or disorder is an acute disease or disorder. In some embodiments, oligosaccharide compositions may be used to treat chronic obstructive pulmonary disease (COPD), asthma, bronchiectasis, cystic fibrosis, pulmonary fibrosis, interstitial lung disease, tuberculosis, acute bronchitis (e.g., a chest cold), chronic bronchitis or allergy in subjects (e.g., human subjects). In some embodiments, oligosaccharide compositions may be used to treat chronic obstructive pulmonary disease (COPD). In some embodiments, oligosaccharide compositions may be used to treat asthma. Further, in some embodiments, oligosaccharide compositions may be used to treat a subject having dysbiosis of the lung microbiome. In some embodiments, oligosaccharide compositions may be used to reduce pathogen (e.g., CRE or VRE) levels and/or pathogen colonization in subjects having an inflammatory lung disease or disorder, e.g., to prevent or treat pathogenic infections (e.g., compostions used to prevent a secondary infection, e.g., of the lungs or the gut). In some embodiments, oligosaccharide compositions may be used to reduce pathogen (e.g., viral pathogens, such as, e.g., respiratory virus) levels and/or pathogen colonization in subjects having an inflammatory lung disease or disorder, e.g., to prevent or treat pathogenic infections. In some embodiments, the oligosaccharide composition is formulated as powder, e.g., for reconstitution (e.g., in water) for oral administration. In some embodiments, the oligosaccharide composition is formulated as a pharmaceutical composition. In some embodiments, the oligosaccharide composition is formulated as a pharmaceutical composition for administration to the gastrointestinal tract (e.g., the intestines, e.g., the large intestine). In some embodiments, the oligosaccharide composition is administered in addition to or in combination with use of a standard of care treatment (e.g., standard of care treatment for inflammatory lung diseases, e.g., probiotics, short-chain fatty acid sources, bronchodilators, antibiotics and corticosteroids, etc.).

In some embodiments, oligosaccharide compositions provided herein effectively treat a subject having or suspected of having an inflammatory lung disease or disorder. In some embodiments, oligosaccharide compositions provided herein effectively treat a subject who is suffering or recovering from an inflammatory episode (e.g., episodes of acute exacerbations of COPD (AECOPD) or an asthma attack) from 1-14 days, 3-12 days, or 5-10 days from the onset of the episode. In some embodiments, oligosaccharide compositions provided herein effectively treat a subject who has a mild clinical presentation and is able to manage their illness at home (e.g., in an outpatient setting, without hospitalization). In some embodiments, treatment of an inflammatory lung disease or disorder in a subject results in reversal of the disease or disorder. In some embodiments, treatment of an inflammatory lung disease or disorder in a subject prevents the hospitalization of a subject. In some embodiments, treatment of an inflammatory lung disease or disorder in a subject reduces the likelihood that the subject will need to be hospitalized. In some embodiments, treatment of an inflammatory lung disease or disorder in a subject minimizes or prevents progression of the disease (e.g., progression from mild or moderate symptoms to severe symptoms, e.g., such that subject requires further intervention).

In some embodiments, oligosaccharide compositions provided herein effectively treat chronic obstructive pulmonary disease (COPD). COPD is a lung disease characterized by chronic obstruction of lung airflow that interferes with normal breathing and is not fully reversible. In some embodiments, COPD is associated with emphysema and/or chronic bronchitis. Emphysema involves the destruction of alveoli at the end of bronchioles of the lungs. Chronic bronchitis is inflammation of the lining of the bronchial tubes and is characterized by persistent cough and excess sputum production.

Eosinophils contribute to inflammation that promotes airway obstruction in patients with COPD. Airway neutrophilia is a common feature of many chronic inflammatory lung diseases and is associated with disease progression, often regardless of the initiating cause. Neutrophils and their products are thought to be key mediators of the inflammatory changes in the airways of patients with chronic obstructive pulmonary disease and have been shown to cause many of the pathological features associated with disease, including emphysema and mucus hypersecretion. In some embodiments, oligosaccharide compositions provided herein reduce the number of (infiltrating) eosinophils and/or neutrophils present in the tissue of an inflamed site, e.g., the lung, thereby reducing inflammation.

Lung airflow obstruction caused by COPD is, in some embodiments, clinically measured by spirometry and expressed in units of Forced Expiratory Volume in the first second of exhalation (FEV1). The four-point Global Initiative for Chronic Obstructive Lung Disease (GOLD) scale is a common classifier of COPD severity which ranges from GOLD I (mild; FEV1 equal to or greater than 80%) to GOLD IV (very severe status; FEV1 equal to or less than 30%). Patients with GOLD III or IV status are more susceptible to episodes of acute exacerbations (AECOPD) which require urgent medical treatment and, in severe cases, hospitalization.

Accordingly, in some embodiments, oligosaccharide compositions described herein are effective in treating moderate COPD (e.g., GOLD Stage II COPD). A subject having GOLD Stage II COPD has a Forced Expiratory Volume in the first second of exhalation (FEV1) of greater than 50% but less than 80% (e.g., 50-80%). In some embodiments, oligosaccharide compositions described herein are effective in treating severe COPD (e.g., GOLD Stage III COPD). A subject having GOLD Stage III COPD has a Forced Expiratory Volume in the first second of exhalation (FEV1) of greater than 30% but less than 50% (e.g., 31-49%). In some embodiments, oligosaccharide compositions described herein are effective in treating very severe COPD (e.g., GOLD Stage IV). A subject having GOLD Stage II COPD has a Forced Expiratory Volume in the first second of exhalation (FEV1) of equal to or less than 30%.

About 78% of AECOPDs are associated with respiratory viral and/or bacterial infections with the remainder due to over-activation of lung eosinophilic cell-types or pauci-inflammatory responses. Increases in the bacterial pathogens Moraxella catarrhalis and Haemophilus influenzae have, in particular, been associated with COPD severity and bronchiectasis. The most common respiratory viruses associated with AECOPDs are influenza (FLU), human rhinovirus (HRV) and respiratory syncytial virus (RSV). Administration of the oligosaccharide compositions described herein are effective in reducing the relative and/or absolute abundance of bacterials pathogens such as Moraxella catarrhalis and Haemophilus influenzae; and/or preventing bacterial infections. In other embodiments, administration of the oligosaccharide compositions described herein are effective in reducing viral titers of influenza (FLU), human rhinovirus (HRV), respiratory syncytial virus (RSV) and coronaviruses; and/or preventing respiratory viral infections.

Recent studies have shown that the lung is not sterile and, like the intestinal tract, supports a dynamic microbiome. Moreover, dysbiosis of the lung microbiome is associated with several inflammatory lung diseases including asthma, cystic fibrosis, bronchiectasis and COPD. In particular, multiple studies show significant, specific lung microbiome dysbiosis during AECOPD exacerbations in COPD patients which are highly associated with the occurrences of respiratory pathogens (e.g., fungal, viral and/or bacterial).

In some embodiments, oligosaccharide compositions provided herein effectively treat a dysbiosis of the lung microbiome. In some embodiments, a dysbiosis of the lung microbiome is a shifting of the lung microbiota such that the relative or absolute abundance of one or more non-desired taxa (e.g., pathogenic taxa or pathobionts) has increased and/or the relative or absolute abundance of one or more desired taxa (e.g., commensal taxa) has decreased. Administration of the oligosaccharide composition can modulate the abundance of the desired and/or non-desired bacterial taxa in the subject's gastrointestinal microbiota, thereby treating the dysbiosis.

A subject having severe or very severe COPD (e.g., GOLD Stage III or IV COPD) is more susceptible to episodes of acute exacerbations of COPD (AECOPD) relative to a healthy subject or a subject having GOLD Stage I COPD or GOLD Stage II COPD. In some embodiments, a subject having severe or very severe COPD has a higher frequency or severity of AECOPDs. In some embodiments, a subject having COPD experiences at least one, two, or more AECOPD episodes per year. In some embodiments, the AECOPD episodes have a duration of 5, 6, 7, 8, 9, 10, or more days. In some embodiments, a subject having severe or very severe COPD has longer recovery times after experiencing an AECOPD relative to a subject having GOLD Stage I COPD or GOLD Stage II COPD who has experienced an AECOPD. In some embodiments, a subject having severe or very severe COPD experiences at least one, two, or three AECOPD episodes per year that result in hospitalization of the subject.

In some embodiments, oligosaccharide compositions provided herein effectively treat asthma. Asthma is a disorder characterized by a narrowing of the lung airways and excessive sputum production. A subject having asthma may have difficulty breathing, have a persistent cough, and/or shortness of breath, among other symptoms. In some embodiments, a subject having asthma experiences episodic asthma attacks that require therapeutic intervention (e.g., bronchodilators) and/or hospitalization.

In some embodiments, oligosaccharide compositions provided herein effectively reduce the frequency or severity of an inflammatory episode (e.g. AECOPD or asthma attack). In some embodiments, the subject experiences fewer inflammatory episodes (e.g. AECOPD or asthma attack) per year following administration of the oligosaccharide composition relative to a control subject (e.g., untreated subject) or a baseline measurement. In some embodiments, oligosaccharide compositions provided herein effectively reduces the duration of an inflammatory episode (e.g., AECOPD or asthma attack) by at least 10%, 20%, 30%, 40%, 50%, 60%, 70%, 75%, 80%, 85%, or more relative to an average AECOPD duration prior to the administration.

In some embodiments, oligosaccharide compositions provided herein effectively prevent the hospitalization of a subject having or suspected of having an inflammatory lung disease or disorder (e.g., in response to an inflammatory episode such as an AECOPD). In some embodiments, oligosaccharide compositions provided herein reduce the likelihood that a subject having or suspected of having an inflammatory lung disease or disorder will need to be hospitalized (e.g., in response to an inflammatory episode such as an AECOPD), thereby reducing, e.g., the number or frequency of required hospitalizations.

In some embodiments, oligosaccharide compositions provided herein effectively prevents the hospitalization of at least 1 out of every 100 subjects having an AECOPD, at least 5 out of every 100 subjects, at least 10 out of every 100 subjects, at least 20 out of every 100 subjects, at least 30 out of every 100 subjects, at least 40 out of every 100 subjects, at least 50 out of every 100 subjects, at least 60 out of every 100 subjects, at least 70 out of every 100 subjects, at least 80 out of every 100 subjects, at least 90 out of every 100 subjects, or at least 95 out of every 100 subjects. In some embodiments, oligosaccharide compositions provided herein effectively prevents the hospitalization of 10-100%, 10-20%, 15-25%, 20-50%, 40-60%, 50-75%, 60-80%, 75-90%, 80-100%, or 90-100% of subjects having an AECOPD who would otherwise require hospitalization.

In some embodiments, oligosaccharide compositions provided herein effectively reduces the length of hospitalization (e.g., by 1-3, 1-5, 1-10, 3-5, 3-10, or 5-15 days) of at least 1 out of every 100 subjects having an AECOPD, at least 5 out of every 100 subjects, at least 10 out of every 100 subjects, at least 20 out of every 100 subjects, at least 30 out of every 100 subjects, at least 40 out of every 100 subjects, at least 50 out of every 100 subjects, at least 60 out of every 100 subjects, at least 70 out of every 100 subjects, at least 80 out of every 100 subjects, at least 90 out of every 100 subjects, or at least 95 out of every 100 subjects, relative to a control subject or population of subjects (e.g., a subject or population of subjects not receiving the oligosaccharide composition). In some embodiments, oligosaccharide compositions provided herein effectively reduces the length of hospitalization (e.g., by 1-3, 1-5, 1-10, 3-5, 3-10, or 5-15 days) of 10-100%, 10-20%, 15-25%, 20-50%, 40-60%, 50-75%, 60-80%, 75-90%, 80-100%, or 90-100% of subjects, relative to a control subject or population of subjects (e.g., a subject or population of subjects not receiving the oligosaccharide composition).

In some embodiments, oligosaccharide compositions provided herein effectively reduce the time (e.g., the average time or the median time) to resolution of symptoms in a subject having an episode relating to an inflammatory lung disease (e.g., an AECOPD), optionally combined with a standard of care treatment (e.g., standard of care treatment for inflammatory lung diseases, e.g., bronchodilators, antibiotics and corticosteroids, etc.), compared to a control subject (e.g., a subject not receiving the oligosaccharide compositions provided herein) optionally receiving standard of care. In some embodiments, a standard of care treatment includes bronchodilators, antibiotics and corticosteroids. In some embodiments, a standard of care treatment is triple therapy (e.g., administration of long-acting muscarinic antagonists (LAMAs), long-acting inhaled B-agonists (LABAs) and inhaled corticosteroids (ICSs)). In some embodiments, a standard of care treatment is dual bronchodilation (LAMAs and LABAs). In some embodiments, a standard of care treatment has been administered to a subject for at least 1, 2, 3, 4, 5, 6, 9, 12, 15, 18, 24 or more months prior to administration of the oligosaccharide composition. In some embodiments, oligosaccharide compositions provided herein effectively reduce the time (e.g., the average time or median time) to resolution of symptoms by 10-100%, 10-20%, 15-25%, 20-50%, 40-60%, 50-75%, 60-80%, 75-90%, 80-100%, or 90-100%. In some embodiments, the symptoms resolved comprise (e.g., consist) of cough, chills and/or repeated shaking with chills, muscle pain, fever, headache, anosmia/ageusia, shortness of breath, and sore throat. In some embodiments, the symptoms resolved comprise (e.g., consist) of persistent cough, coughs that include excess mucus, shortness of breath, wheezing or squeaking while breathing, chest tightness, frequent colds or bacterial infections, low energy, sudden weight loss, and/or swelling of extremities (e.g., ankles, feet, legs). In some embodiments, the subject or subjects have at least one (e.g., at least 1, 2, or 3) comorbidity condition. In some embodiments, the subject or subjects do not have a comorbidity condition. In some embodiments, comorbidity conditions include, but are not limited to, diabetes mellitus, cardiovascular disease, hypertension, renal disease (e.g., chronic renal disease), liver disease (e.g., chronic liver disease), an immunocompromised condition, cancer, a neurologic disorder, stroke, or other chronic disease.

In some embodiments, oligosaccharide compositions provided herein effectively prevent a secondary infection (e.g., secondary infection of the lungs, urinary tract or the gut) in a subject having or suspected of having an inflammatory lung disease. In some embodiments, a subject having or suspected of having an inflammatory lung disease who receives an oligosaccharide composition is about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100% less likely to develop a secondary infection than a control subject (e.g., untreated subject).

In some embodiments, oligosaccharide compositions provided herein effectively prevents a secondary infection in at least 1 out of every 100 subjects, at least 5 out of every 100 subjects, at least 10 out of every 100 subjects, at least 20 out of every 100 subjects, at least 30 out of every 100 subjects, at least 40 out of every 100 subjects, at least 50 out of every 100 subjects, at least 60 out of every 100 subjects, at least 70 out of every 100 subjects, at least 80 out of every 100 subjects, at least 90 out of every 100 subjects, or at least 95 out of every 100 subjects. In some embodiments, oligosaccharide compositions provided herein effectively a secondary infection in 10-100%, 10-20%, 15-25%, 20-50%, 40-60%, 50-75%, 60-80%, 75-90%, 80-100%, or 90-100% of subjects.

A secondary infection (e.g., secondary infection of the lung, urinary tract or gut) may be a fungal infection or a bacterial infection. In some embodiments, a secondary infection is caused by any pathogen (e.g., bacterial pathogen or fungal pathogen) as described herein. In certain embodiments, a bacterial infection is caused by a drug or antibiotic resistant bacteria (e.g., vancomycin resistant Enterococcus (VRE) or carbapenem resistant Enterobacteriaceae (CRE)). In certain embodiments, a secondary infection is caused by Mycobacterium tuberculosis, mycobacteria, Streptococcus pneumoniae, Haemophilus influenzae, Moraxella catarrhalis, mycoplasma, Staphylococcus aureus, or Pseudomonas. In some embodiments, a secondary infection is caused by Clostridium difficile. In some embodiments, a secondary infection is caused by vancomycin resistant Enterococcus (VRE). In some embodiments, a secondary infection is caused by carbapenem resistant Enterobacteriaceae (CRE). In some embodiments, a secondary infection is caused by a fungal pathogen. In some embodiments, a secondary infection is caused by Candida albicans. In some embodiments, a secondary infection is caused by Candida glabrata. In some embodiments, a secondary infection is caused by Candida krusei. In some embodiments, a secondary infection is caused by Candida tropicalis.

In some embodiments, pathogens that may cause a secondary infection includes bacterial pathogens (e.g., Abiotrophia spp., (e.g., A. defective), Achromobacter spp., Acinetobacter spp., (e.g., A. baumanii), Actinobaculum spp., (e.g., A. schallii), Actinomyces spp., (e.g., A. israelii), Aerococcus spp., (e.g., A. urinae), Aeromonas spp., (e.g., A. hydrophila), Aggregatibacter spp., e.g. A. aphrophilus, Bacillus anthracis, Bacillus cereus group, Bordetella spp., Brucella spp., e.g. B. henselae, Burkholderia spp., e.g., B. cepaciae, Campylobacter spp., e.g., C. jejuni, Chlamydia spp., Chlamydophila spp., Citrobacter spp., e.g., C. freundii, Clostridium botulinum, Clostridium difficile, Clostridium perfringens, Corynebacterium spp., e.g., C. amycolatum, Cronobacter, e.g., C. sakazakii, Enterobacteriaceae, including many of the genera below, Ehrlichia spp., Enterobacter spp., e.g., E. cloacae, Enterococcus spp., e.g. E. faecium, Escherichia spp., including enteropathogenic, uropathogenic, and enterohemorrhagic strains of E. coli, Francisella spp., e.g. F. tularensis, Fusobacterium spp., e.g. F. necrophorum, Gemella spp., e.g. G. mobillorum, Granulicatella spp., e.g. G. adiaciens, Haemophilus spp., e.g. H. influenza, Helicobacter spp., e.g. H. pylori, Kingella spp., e.g. K. kingae, Klebsiella spp., e.g. K. pneumoniae, Legionella spp., e.g. L. pneumophila, Leptospira spp., Listeria spp., e.g. L. monocytogenes, Morganella spp., e.g. M. morganii, Mycobacterium spp., e.g. M. abcessus, Neisseria spp., e.g. N. gonorrheae, Nocardia spp., e.g. N. asteroids, Ochrobactrum spp., e.g. O. anthropic, Pantoea spp., e.g. P. agglomerans, Pasteurella spp., e.g. P. multocida, Pediococcus spp., Plesiomonas spp., e.g. P. shigelloides, Proteus spp., e.g. P. vulgaris, Providencia spp., e.g. P. stuartii, Pseudomonas spp., e.g. P. aeruginosa, Raoultella spp., e.g. R. ornithinolytica, Rothia spp., e.g. R. mucilaginosa, Salmonella spp., e.g. S. enterica, Serratia spp., e.g. S. marcesens, Shigella spp., e.g. S. flexneri, Staphylococcus aureus, Staphylococcus lugdunensis, Staphylococcus pseudintermedius, Staphylococcus saprophyticus, Stenotrophomonas spp., e.g. S. maltophilia, Streptococcus agalactiae, Streptococcus anginosus, Streptococcus constellatus, Streptococcus dysgalactiae, Streptococcus intermedius, Streptococcus milleri, Streptococcus pseudopneumoniae, Streptococcus pyogenes, Streptooccus pneumoniae, Treponema spp., Ureaplasma ureolyticum, Vibrio spp., e.g. V. cholerae, and Yersinia spp., (e.g., Y. enterocolitica)); viral pathogens (e.g., Adenovirus, Astrovirus, Cytomegalovirus, Enterovirus, Norovirus, Rotavirus, and Sapovirus); and gastrointestinal protozoan pathogens (e.g., Cyclospora spp., Cryptosporidium spp., Entamoeba histolytica, Giardia lamblia, and Microsporidia, (e.g., Encephalitozoon canaliculi)).

In some embodiments, a subject having an inflammatory lung disease or disorder is more susceptible to a respiratory viral infection. In some embodiments, a viral infection is caused by a rhinovirus, coronavirus, enterovirus, adenovirus, parainfluenza virus, respiratory syncytial virus, influenza virus, or measles virus. In some embodiments, a viral infection is caused by one or more of influenza (FLU), human rhinovirus (HRV), respiratory syncytial virus (RSV) and coronaviruses (Family Coronaviridae).

In some embodiments, a coronavirus is an alphacoronavirus, a betacoronavirus, a gammacoronavirus, or a deltacoronavirus. In some embodiments, a coronavirus is selected from the group consisting of: severe acute respiratory syndrome-related coronavirus (SARS-COV, SARS-COV-2), human coronavirus 229E, human coronavirus NL63, miniopterus bat coronavirus 1, miniopterus bat coronavirus HKU8, porcine epidemic diarrhea virus, rhinolophus bat coronavirus HKU2, scotophilus bat coronavirus 512, tacoronavirus 1, human coronavirus HKU1, murine coronavirus, pipistrellus bat coronavirus HKU5, rousettus bat coronavirus HKU9, tylonycteris bat coronavirus HKU4, Middle East respiratory syndrome-related coronavirus, and hedgehog coronavirus 1 (EriCoV). In some embodiments, a coronavirus is a severe acute respiratory syndrome-related coronavirus. In some embodiments, a severe acute respiratory syndrome-related coronavirus is a SARS-COV-2 strain. In some embodiments, a coronavirus infection is COVID-19.

In some embodiments, administration of an oligosaccharide composition reduces the severity of an inflammatory lung disease or disorder (e.g., COPD). In some embodiments, the severity of an inflammatory lung disease or disorder is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100% relative to a control subject (e.g., untreated subject).

In some embodiments, administration of an oligosaccharide composition reduces the severity of the symptoms of an inflammatory lung disease or disorder. In some embodiments, the severity of the symptoms of an inflammatory lung disease or disorder are reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100% relative to a control subject (e.g., untreated subject).

In some embodiments, a subject who has been administered an oligosaccharide composition experiences a reduction of the severity of any one individual symptom relative to a control subject (e.g., untreated subject, a subject treated with a standard of care therapy) or a baseline measurement. In some embodiments, the subject has a reduction in the severity of a persistent cough, coughs that include excess mucus, shortness of breath, wheezing or squeaking while breathing, chest tightness, frequent colds or bacterial infections, low energy, sudden weight loss, and/or swelling of extremities (e.g., ankles, feet, legs). In some embodiments, the severity of a symptom may be rated (i.e., scored) on a scale of 0-3, where 0 means that the symptom is absent, 1 means that the symptom is mild (present but easily tolerated), 2 means that the symptom is moderately severe (bothersome but tolerable), and 3 means that the symptom is very severe (hard to tolerate; interferes considerably with daily activity). In some embodiments, a subject who has been administered an oligosaccharide composition rates the severity of at least one symptom to be lowered following the administration of the oligosaccharide composition. In some embodiments, the subject experiences a reduction of the severity of 2, 3, 4, 5, 6, or more symptom scores. In some embodiments, the subject who has been administered an oligosaccharide composition experiences a reduction of the severity of a plurality (e.g., a majority, nearly all, or all) of symptoms associated with an inflammatory lung disease. In some embodiments, the reduction of the severity in a plurality (e.g., a majority, nearly all, or all) of symptoms is modest, e.g., a reduction of about 1 or about 2 on a 0-3 scale, wherein the plurality of symptoms may persist after treatment in a milder manner. Without wishing to be bound by theory, by acting to modulate the immune response of a subject having an inflammatory lung disease or disorder, the oligosaccharide compositions provided herein may act in a generalized manner against the symptoms of the disease or disorder, providing relief across symptoms (e.g., rather than targeted relief of a single symptom).

The oligosaccharide composition may be administered to the subject on a daily, weekly, biweekly, or monthly basis. In some embodiments, the composition is administered to the subject more than once per day (e.g., 2, 3, or 4 times per day). In some embodiments, the composition is administered to the subject once or twice per day for one, two, three, or four weeks in a row.

In some embodiments, the composition is administered to the subject according to the following schedule: 18 grams on days 1 and 2 of a treatment protocol; 36 grams on days 3 and 4 of a treatment protocol; and 72 grams on days 5-14 of a treatment protocol.

In some embodiments, an effective amount of an oligosaccharide is a total of 5-200 grams, 5-150 grams, 5-100 grams, 5-75 grams, 5-50 grams, 5-25 grams, 10-50 grams, 25-50 grams, 30-60 grams, 50-75 grams, 50-100 grams, 18-72 grams, or 36-72 grams administered daily.

The oligosaccharide composition of the disclosure is well tolerated by a subject (e.g., oligosaccharide compositions do not cause or cause minimal discomfort, e.g., production of gas or gastrointestinal discomfort, in subjects). In some embodiments, 5-200 grams, 5-150 grams. 5-100 grams, 5-75 grams, 5-50 grams, 5-25 grams, 10-50 grams, 25-50 grams, 30-60 grams, 50-75 grams, 50-100 grams, 18-72 grams, or 36-72 grams of total daily dose are well tolerated by a subject. The amount of an oligosaccharide composition that is administered to the subject at a single time or in a single dose is well tolerated by the subject.

In some embodiments, the amount of the oligosaccharide composition that is administered to the subject at a single time or in a single dose is more tolerated by the subject than a similar amount of commercial low-digestible sugars such as fructooligosaccharides (FOS). Commercial low-digestible sugars are known in the art to be poorly tolerated in subjects (See, e.g., Grabitske, H. A., Critical Reviews in Food Science and Nutrition, 49:327-360 (2009)), e.g., at high doses. For example, tolerability studies of FOS indicate that 20 grams FOS per day causes mild gastrointestinal symptoms and that 30 grams FOS per day causes major discomfort and gastrointestinal symptoms.

In some embodiments, oligosaccharide compositions provided herein effectively promotes an increased relative or absolute abundance of anti-inflammatory metabolites in the subject relative to a baseline measurement or control. In some embodiments, this increased abundance is in the lungs, sputum and/or gut of the subject. The anti-inflammatory metabolites may be short-chain fatty acids (SCFAs), indoles, tryptophan and derivatives thereof, and/or small molecule modulators of Aryl Hydrocarbon Receptor (AHR) and/or IL-22. In some embodiments, oligosaccharide compositions provided herein cause bacterial organisms in the gut microbiome to produce an increase in anti-inflammatory metabolites of at least 1%, at least 2%, at least 3%, at least 4%, at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to control (e.g., treatment of bacteria with water).

In some embodiments, oligosaccharide compositions provided herein effectively promotes an increased amount of short-chain fatty acids (SCFAs) in the gut microbiome of the subject. In some embodiments, the amount of short-chain fatty acids in the gut microbiome of the subject are increased by at least 2-fold (e.g., at least 3-fold, at least 4-fold, at least 5-fold) following the administration of the oligosaccharide composition. Short chain fatty acids include acetate, propionate, and butyrate. In some embodiments, oligosaccharide compositions provided herein cause bacterial organisms in the gut microbiome to produce an increased amount of short-chain fatty acids. In some embodiments, oligosaccharide compositions provided herein cause bacterial organisms in the gut microbiome to produce an increase in SCFAs of at least 1%, at least 2%, at least 3%, at least 4%, at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to control (e.g., treatment of bacteria with water). In some embodiments, oligosaccharide compositions provided herein cause bacterial organisms in the gut microbiome to produce an increase in SCFAs of at least 1.5-fold, 2-fold, 3-fold, 4-fold, 5-fold, 6-fold, 7-fold, 8-fold, 9-fold, or 10-fold relative to control (e.g., treatment of bacteria with water). In some embodiments, oligosaccharide compositions provided herein cause bacterial organisms in the gut microbiome to produce at least 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, or 50 mM of total SCFA (e.g., as measured in a fecal sample). In some embodiments, oligosaccharide compositions provided herein cause bacterial organisms in the gut microbiome to produce at least 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, or 30 mM of total acetate (e.g., as measured in a fecal sample). In some embodiments, oligosaccharide compositions provided herein cause bacterial organisms in the gut microbiome to produce at least 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, or 30 mM of total propionate (e.g., as measured in a fecal sample). In some embodiments, oligosaccharide compositions provided herein cause bacterial organisms in the gut microbiome to produce at least 0.3 mM, 0.4 mM, 0.5 mM, 0.6 mM, 0.7 mM, 0.8 mM, 0.9 mM, 1 mM, 1.25 mM, 1.5 mM, 1.75 mM, 2 mM, 2.25 mM, 2.5 mM, 2.75 mM, or 3 mM of total butyrate (e.g., as measured in a fecal sample).

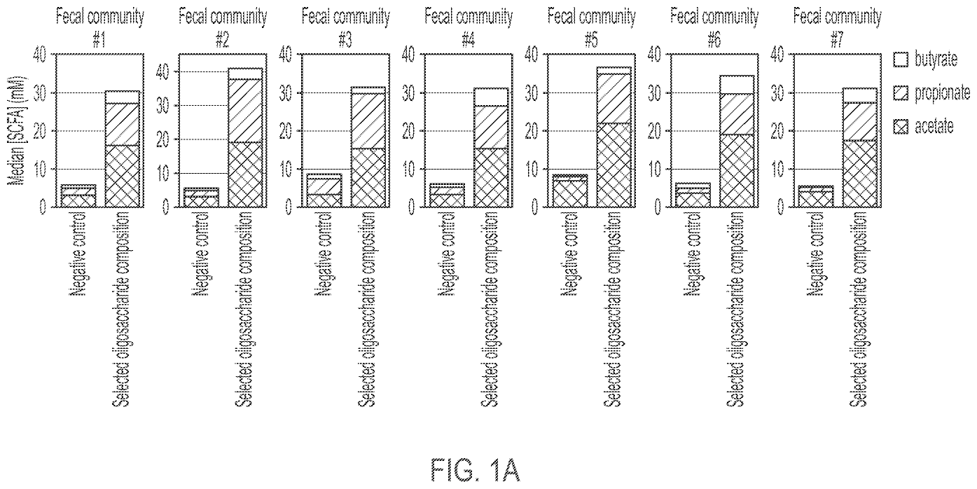

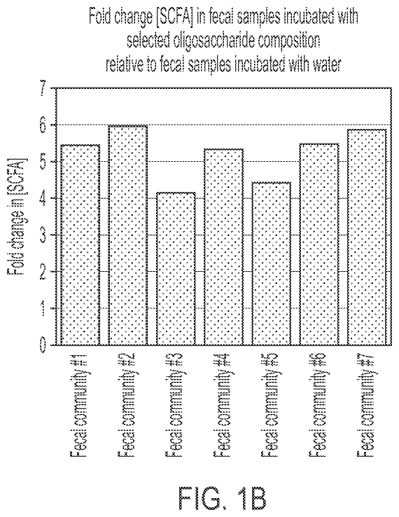

In some embodiments, oligosaccharide compositions provided herein promotes an increased amount of short-chain fatty acids (SCFAs) in the gut microbiome across a plurality of different subjects. Without wishing to be bound by theory, the disclosure is directed in part to the discovery that oligosaccharide compositions provided herein are capable of promoting an increased amount of SCFAs in the gut microbiome in a subject non-specific manner, meaning, e.g., that the oligosaccharide compositions may be useful to a wide segment of potential patients rather than a single individual (or small set of individuals) having a specific microbiome composition. Sec, e.g., Example 6 and FIGS. 1A-1B. In some embodiments, an oligosaccharide composition provided herein is capable of promoting an increased amount of SCFAs in the gut microbiome of at least 2, 3, 4, 5, 6, 7, 8, 9, 10, 15, 20, 50, or 100 different subjects (e.g., as demonstrated by an increased amount of SCFAs after ex vivo fermentation of selected oligosaccharide composition by fecal communities from at least 2, 3, 4, 5, 6, or 7 different subjects). In some embodiments, an oligosaccharide composition provided herein is capable of promoting an increased amount of SCFAs in the gut microbiome of at least 2, 3, 4, 5, 6, or 7 different subjects, e.g., at least 2 or 7 different subjects.

In some embodiments, oligosaccharide compositions provided herein promote production of a consistent ratio of different SCFAs in the gut microbiome (e.g., across a plurality of different subjects and/or across administrations to an individual subject). Without wishing to be bound by theory, the disclosure is directed in part to the discovery that oligosaccharide compositions provided herein are capable of promoting production of a ratio of SCFAs (e.g., of butyrate, propionate, and acetate) that is distinct and/or improved relative to the ratio promoted by either a baseline (e.g., water) or a control treatment (e.g., treatment with a commercially available oligosaccharide composition (e.g., FOS, GOS, XOS, or PDX). Levels of particular SCFAs can be measured by ex vivo methods, e.g., as described in Example 6. In some embodiments, the oligosaccharide compositions provided herein are capable of promoting production of propionate (e.g., on average, e.g., across a plurality of fecal communities) at a level at least 1.2, 1.3, 1.4, 1.5, 1.75, 2, 2.25, 2.5, 2.75, 3, 3.25, 3.5, 3.75, 4, 4.25, 4.5, 4.75, or 5 fold higher than either negative control treatment (e.g., water) or treatment with a commercially available oligosaccharide composition (e.g., FOS, GOS, XOS, or PDX). In some embodiments, the oligosaccharide compositions provided herein are capable of promoting production of exemplary SCFAs acetate, propionate, and butyrate (e.g., on average, e.g., across a plurality of fecal communities) at a ratio (acetate:propionate:butyrate) of about 9:about 5.6:about 1; about 6:about 4:about 1, or about 54-60% acetate, about 34-40% propionate, and about 6-10% butyrate.

In some embodiments, oligosaccharide compositions provided herein effectively decreases a host immune response of a subject having an inflammatory lung disease or disorder when the subject is subjected to an inflammatory episode or a pathogenic infection. Without wishing to be bound by theory, altering the microbiota with a method or oligosaccharide composition described herein may increase or decrease the level of one or more taxa of the gastrointestinal microbiota, altering the microbiota's capacity to process host metabolites and secreted metabolites of its own, thereby influencing features of a host immune response. In some embodiments, a method or oligosaccharide composition described herein decreases a host immune response of a subject to a viral respiratory illness by at least 2-fold (e.g., at least 3-fold, at least 4-fold, at least 5-fold) following the administration of the oligosaccharide composition (e.g., assessed by measuring a suitable immune/inflammatory biomarker panel (e.g., determining the levels of pro-inflammatory cytokines such as, e.g., IL-1 beta, IL-6, and TNF-alpha and anti-inflammatory cytokines, which include, e.g., IL-4, IL-10, and IL-13, and others, such as, e.g., cytokines, such as, e.g., GM-CSF, IFN alpha, IFN gamma, IL-1 alpha, IL-1 beta, IL-4, IL-6, IL-8, IL-10, IL-12p70, IL-13, IL-17A (CTLA-8), TNF alpha, chemokines, such as, e.g., IP-10 (CXCL10), MCP-1 (CCL2), MIP-1 alpha (CCL3), MIP-1 beta (CCL4), and cell adhesion and inflammatory response markers, such as, e.g., ICAM-1, CD62E (E-selectin), CD62P (P-Selectin)). In some embodiments, levels of pro-inflammatory cytokines are reduced in the lungs, sputum and/or gut of the subject. In some embodiments, the pro-inflammatory cytokines are selected from the group consisting of IL-8, IL-1, IL-10, IL-33, IL-6, IP-10, TSLP, neutrophil elastase, lactoferrin and eosinophil cationic protein (ECP).

In some embodiments, oligosaccharide compositions provided herein effectively promote a decreased amount of a biomarker of inflammation, e.g., in the bloodstream of the subject. In some embodiments, the amount of the biomarker of inflammation in the bloodstream of the subject is decreased by at least 2-fold (e.g., at least 3-fold, at least 4-fold, at least 5-fold) following the administration of the oligosaccharide composition. In some embodiments, the amount of the biomarker of inflammation in the bloodstream of the subject is decreased by at least 10, 25, 50, 75, or 90% following the administration of the oligosaccharide composition. Without wishing to be bound by theory, changes in the abundance of different bacterial populations in the microbiome of a subject can decrease the level of inflammatory cytokines and other inflammatory markers in a subject, which may be especially advantageous in a subject exhibiting a viral respiratory illness and a heightened immune response associated with increased inflammation. In some embodiments, the biomarker of inflammation comprises C Reactive Protein (CRP). In some embodiments, a subject with an inflammatory lung disease or disorder has one or more comorbidities (e.g., associated with chronic inflammation) and the oligosaccharide compositions provided herein effectively promotes a decreased amount of a biomarker of inflammation (e.g., CRP). In some embodiments, levels of biomarkers of inflammation are reduced in the lungs, sputum and/or gut of the subject.

In some embodiments, oligosaccharide compositions provided herein effectively boost the innate immune response of a subject.

Eosinophils and neutrophils contribute to inflammation that promotes airway obstruction in subjects having an inflammatory lung disease or disorder (e.g., COPD). In some embodiments, oligosaccharide compositions described herein are effective in reducing the relative and/or absolute abundance of eosinophils and/or neutrophils, e.g., infiltrating eosinophils and/or neutrophils in an inflamed tissue at a body site, e.g., the lung/lung tissue. In some embodiments, the abundance of eosinophils and/or neutrophils is reduced by at least 10%, 20%, 30%, 40%, 50%, 60%, 70% or more, relative to a control (e.g., untreated subject) or a baseline measurement.

In some embodiments, oligosaccharide compositions provided herein effectively reduce colonization with, prevent colonization with, or reduce the risk of an adverse effect of a pathogen to a subject having an inflammatory lung disease or disorder. In some embodiments, oligosaccharide compositions prevent a secondary pathogenic bacterial infection. In some embodiments, provided is a method of decolonizing the gastrointestinal tract (e.g., all of the GI tract or part of the GI tract, e.g. the small intestine or the large intestine) from a pathogen or an antibiotic resistance gene carrier. In some embodiments, the method comprises shifting the microbial community in the gastrointestinal tract toward a commensal population, e.g., thereby replacing (e.g. outcompeting) a pathogen or an antibiotic resistance gene carrier.

In some embodiments, provided is a method of reducing a pathogen reservoir in a subject having an inflammatory lung disease or disorder by administering an oligosaccharide composition to the subject, e.g., in an effective amount and/or to a sufficient number of subjects that the pathogen reservoir is reduced. In some embodiments, the pathogen reservoir is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard. In some embodiments, a pathogen reservoir may represent about 5%, about 10%, about 15%, about 20%, about 30%, about 35%, about 40%, about 45%, about 50%, about 55%, about 60%, about 65%, about 70%, about 75%, about 80%, or about 85% of the total bacterial reservoir of a subject (e.g., about 5%, about 10%, about 15%, about 20%, about 30%, about 35%, about 40%, about 45%, about 50%, about 55%, about 60%, about 65%, about 70%, about 75%, about 80%, or about 85% of the total bacterial population in the gut or intestines of a subject). In some embodiments, the pathogen reservoir comprises the pathogen biomass. In some embodiments, the bacterial reservoir comprises the total bacterial biomass.

Exemplary pathogens include Enterobacteriaciae (e.g., a family comprising Plesiomonas, Shigella, or Salmonella), Clostridium (e.g., a genus comprising Clostridium difficile), Enterococcus, Staphylococcus (e.g., a genus comprising Staphylococcus aureus), Campylobacter, Vibrio, Aeromonas, Norovirus, Astrovirus, Adenovirus, Sapovirus, or Rotavirus.

In some embodiments, the pathogen is a carbapenem-resistant Enterobacteriaceae (CRE). In some embodiments, the pathogen is a vancomycin-resistant Enterococcus (VRE). In some embodiments, the pathogen is an extended-spectrum beta-lactamase (ESBL) producing organism.

In some embodiments, the pathogen includes Enterobacteriaciae (e.g., a genus comprising Plesiomonas, Shigella, or Salmonella). In some embodiments, the pathogen includes Clostridium (e.g., a genus comprising Clostridium difficile). In some embodiments, the pathogen includes Enterococcus. In some embodiments, the pathogen includes Staphylococcus.

In some embodiments, provided is a method of reducing the rate at which a pathogen causes infection or colonization in a subject having an inflammatory lung disease or disorder by administering a oligosaccharide composition to the subject, e.g., in an effective amount and/or to a sufficient number of subjects that the rate of infection is reduced. In some embodiments, the oligosaccharide composition is administered in an effective amount and/or to a sufficient number of subject(s), that the rate at which a pathogen causes infection, or the severity of pathogen infection, as indicated by assessment of symptoms associated with infection, is reduced. In some embodiments, the rate of infection is reduced by about 1%, about 2%, about 3%, about 4%, about 5%, about 10%, about 15%, about 20%, about 30%, about 35%, about 40%, about 45%, about 50%, about 55%, about 60%, about 65%, about 70%, about 75%, about 80%, about 85%, about 90%, about 95%, about 99%, or about 100%, e.g., relative to a reference standard.

Reduction in the rate of infection or colonization using a method described herein may be prospective or retrospective, e.g., relative to an infection. In some embodiments, the method described herein comprises monitoring a subject or a population of subjects for a similar infection, e.g., through observation of similar symptoms or similar features to those known to be caused by or identified with a pathogen of interest. Rather than, or in addition to using clinical characteristics, any of the methods described herein might be used to more specifically determine the type of the pathogen involved, and its relationship—if any—to spread or a reservoir.

In some embodiments, provided is a method of modulating the gastrointestinal tract (e.g., all of the GI tract or a part thereof, e.g., the small intestine, the large intestine, the colon, and the like) of a subject. In some embodiments, the method comprises modulating the environment (e.g., chemical or physical environment) of the gastrointestinal tract of a subject to make the gastrointestinal tract (and the microbial community therein) less selective or less receptive for a pathogen or an antibiotic resistance gene carrier. In some embodiments, the method further comprises administering a second agent in combination with a oligosaccharide composition, e.g., charcoal or an antibiotic-degrading enzyme (e.g., beta-lactamase), or a synbiotic (e.g., an engineered beta-lactamase (e.g., a non-infectious beta-lactamase).

In some embodiments, provided is a method of managing a secondary pathogenic bacterial infection in a subject having an inflammatory lung disease or disorder. In some embodiments, managing a secondary infection by a pathogen comprises treating, preventing, and/or reducing the risk of developing an infection by a pathogen. In some embodiments, treating a secondary infection by a pathogen comprises administering a oligosaccharide composition to a subject or population having an inflammatory lung disease or disorder.

In some embodiments, the method reduces the abundance of pathogens and increases the relative of abundance of commensal bacteria (e.g., Parabacteroides and Bacteroides), e.g., in a subject, e.g., in the gastrointestinal tract of the subject (e.g, the colon). In some embodiments, the method increases the alpha-diversity (e.g., a high degree of diversity) of a microbial community (e.g., a community of commensal bacteria), e.g., of the gut of a subject.

In some embodiments, oligosaccharide compositions are substantially fermented or consumed by commensal bacteria and are not fermented or consumed by pathogens. In some embodiments, oligosaccharide compositions are substantially fermented or consumed by commensal bacteria and are fermented or consumed by pathogens at low levels. In some embodiments, a oligosaccharide composition that is substantially consumed by commensal bacteria may increase the diversity and biomass of the commensal microbiota and lead to a reduction in the relative abundance of a pathogen(s), such as a bacterial pathogen (e.g., a pathogenic taxa). In some embodiments, a oligosaccharide composition is substantially non-fermented or not consumed by VRE or CRE species. In some embodiments, a oligosaccharide composition is substantially non-fermented or not consumed by C. difficile.

In some embodiments, an oligosaccharide composition supports the growth of commensal or probiotic bacteria, e.g., in a gut microbiome. In some embodiments, a oligosaccharide composition does not support the growth of at least one pathogen, e.g., does not support the growth of a CRE, VRE, and/or C. difficile species.

In some embodiments, administration of an oligosaccharide composition may increase the concentration, amount or relative abundance of commensal bacteria relative to pathogenic bacteria in the microbiome of a subject (e.g., a human patient). In some embodiments, administration of a oligosaccharide composition and a population of viable commensal or probiotic bacteria may increase the concentration, amount, or relative abundance of commensal bacteria relative to pathogenic bacteria in the microbiome of a subject (e.g., a human patient). In some embodiments, administration of a oligosaccharide composition that supports the growth of commensal or probiotic bacteria, e.g., in a gut microbiome, may increase the concentration, amount or relative abundance of commensal bacteria relative to pathogenic bacteria in the microbiome of a subject (e.g., a human patient). In some embodiments, administration of a oligosaccharide composition that does not support the growth of at least one pathogen, e.g., does not support the growth of a CRE, VRE, and/or C. difficile species, e.g., in a gut microbiome, may increase the concentration, amount or relative abundance of commensal bacteria relative to pathogenic bacteria in the microbiome of a subject (e.g., a human patient). In some embodiments, administration of a oligosaccharide composition that supports the growth of commensal or probiotic bacteria and does not support the growth of at least one pathogen, e.g., does not support the growth of a CRE, VRE, and/or C. difficile species, e.g., in a gut microbiome, may increase the concentration, amount or relative abundance of commensal bacteria relative to pathogenic bacteria in the microbiome of a subject (e.g., a human patient).

In some embodiments, administration of an oligosaccharide composition may increase the concentration, amount or relative abundance of Bacteroidetes (e.g., Bacteroidales) relative to pathogenic bacteria in the microbiome of a subject (e.g., a human patient).

In some embodiments, administration of an oligosaccharide composition may increase the concentration, amount or relative abundance of Parabacteroides (e.g., Parabacteroides distasonis and Parabacteroides merdae) and Bacteroides (e.g., Bacteroides thetaiotaomicron, Bacteroides uniformis, Bacteroides caccae) relative to pathogenic bacteria in the microbiome of a subject (e.g., a human patient).

In embodiments, an oligosaccharide composition described herein is co-administered with commensal or probiotic bacterial taxa and bacteria that are generally recognized as safe (GRAS) or known commensal or probiotic microbes. In some embodiments, probiotic or commensal bacterial taxa (or preparations thereof) may be administered to a subject before or after administration of an oligosaccharide composition to the subject. In some embodiments, probiotic or commensal bacterial taxa (or preparations thereof) may be administered to a subject simultaneously with administration of an oligosaccharide composition to the subject.

In some embodiments, an oligosaccharide composition described herein is administered with a population of Bacteroidetes. In some embodiments, an oligosaccharide composition described herein is administered with a population of Bacteroidales. In some embodiments, an oligosaccharide composition is administered in combination with a population of Lactobacillus and/or Akkermansia.

A commensal or probiotic bacteria is also referred to a probiotic. Probiotics can include the metabolites generated by the probiotic bacteria during fermentation. These metabolites may be released to the medium of fermentation, e.g., into a host organism (e.g., subject), or they may be stored within the bacteria. Probiotic bacteria includes bacteria, bacterial homogenates, bacterial proteins, bacterial extracts, bacterial ferment supernatants and combinations thereof, which perform beneficial functions to the host animal, e.g., when given at a therapeutic dose.

Useful probiotics include at least one lactic acid and/or acetic acid and/or propionic acid producing bacteria, e.g., microbes that produce lactic acid and/or acetic acid and/or propionic acid by decomposing carbohydrates such as glucose and lactose. Preferably, the probiotic bacteria is a lactic acid bacterium. In embodiments, lactic acid bacteria include Lactobacillus, Leuconostoc, Pediococcus, Streptococcus, and Bifidobacterium. Suitable probiotic bacteria can also include other bacterias which beneficially affect a host by improving the hosts intestinal microbial balance, such as, but not limited to yeasts such as Saccharomyces, Debaromyces, Candida, Pichia and Torulopsis, molds such as Aspergillus, Rhizopus, Mucor, and Penicillium and Torulopsis, and other bacteria such as but not limited to the genera Bacteroides, Clostridium, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, and Oenococcus, and combinations thereof. In some embodiments, probiotic organisms include Parabacteroides and Bacteroides.

The commensal or probiotic bacteria can be used as a single strain or a combination of multiple strains, wherein the total number of bacteria in a dose of probiotic bacteria is from about 1×10³ to about 1×10¹⁴, or from about 1×10 to about 1×10¹², or from about 1×10⁷ to about 1×10¹¹ CFU per dose.

The probiotic bacterias can be used in a powdered, dry form. The probiotic bacterias can also be administered in the oligosaccharide composition or in a separate oligosaccharide composition, administered at the same time or different time as the oligosaccharide compositions.

In some embodiments, a subject having an inflammatory lung disease or disorder treated by a method described herein further has at least one comorbidity condition. As used herein, a comorbidity condition refers to a medical or health condition (e.g., a disease, disorder, syndrome, or previously resolved version of any of the preceding; or a medical operation (e.g., surgery)), past or present, that may detrimentally affect a subject's prognosis regarding a present or future illness. Without wishing to be bound by theory, a subject having at least one comorbidity condition is thought to be at greater risk of more severe and/or more prolonged symptoms of episodes of inflammatory lung diseases or disorders (e.g., AECOPD), as well as greater risk of adverse effects after recovery from such episodes. In some embodiments, the presence of one or more comorbidity conditions in a subject lengthens the time to resolution of symptoms of such episodes (e.g., the median time to resolution of symptoms in a population of subjects). In some embodiments, comorbidity conditions include diabetes mellitus, cardiovascular disease, hypertension, renal disease (e.g., chronic renal disease), liver disease (e.g., chronic liver disease), an immunocompromised condition, cancer, a neurologic disorder, stroke, being overweight, obesity, or other chronic disease. In some embodiments, a subject has at least one, two, or at least three comorbidity conditions. In some embodiments, a subset of the comorbidity conditions described herein are considered high-risk comorbidities, which carry a higher risk of more severe and/or more prolonged symptoms of infection relative to other comorbidities, as well as greater risk of adverse effects after recovery from infection relative to other comorbidities. In some embodiments, high-risk comorbidities include diabetes mellitus, cardiovascular disease, hypertension, chronic renal disease, or cancer. In other embodiments, a subject having a disease or disorder treated by a method described herein does not have a comorbidity condition (e.g., and still benefits from treatment with a method or oligosaccharide composition described herein).

In some embodiments, being overweight or obese is expressed by body mass index (BMI). In some embodiments, an overweight subject has a BMI of at least 25, 26, 27, 28, or 29, and optionally less than 30, e.g., 25-29.9. In some embodiments, an obese subject has a BMI of at least 30, 31, 32, 33, 34, 35, 36, 37, 38, 39, or 40, e.g., at least 30 or at least 35.

In some embodiments, a subject treated by a method described herein is middle-aged, senior, or elderly. Such subjects are referred to herein as having an age-related risk factor. Without wishing to be bound by theory, subjects having an age-related risk factor are thought to be at greater risk of more severe and/or more prolonged symptoms of an inflammatory lung disease or disorder, as well as greater risk of adverse effects after recovery from illness. In some embodiments, a subject having an age-related risk factor is at least 40, 45, 50, 55, 60, 65, 70, 75, or 80 years old, e.g., at least 45, 55, or 65 years old. In some embodiments, oligosaccharide compositions provided herein effectively reduce one or more symptoms or reduce the time to resolution of one or more symptoms (e.g., the average time) in a subject having an age-related risk factor (e.g., a subject who is at least 45 years old). In some embodiments, a subject having an age-related risk factor is at a greater risk of more severe and/or more prolonged symptoms, as well as greater risk of adverse effects after recovery from an inflammatory episodes.

In some embodiments, a subject treated by a method described herein is a young child or an infant (e.g., less than 12, 11, 10, 9, 8, 7, 6, 5, 4, 3, 2, or 1 month in age). In some embodiments, a young child or an infant is at a greater risk of more severe and/or more prolonged symptoms, as well as greater risk of adverse effects after recovery from an inflammatory episode.

In some embodiments, a subject treated by a method described herein is immunocompromised. An immunocompromised subject refers to any subject whose immune system is less able or unable to combat infection relative to a non-immunocompromised subject. In some embodiments, an immunocompromised subject is a cancer patient, a transplant recipient (e.g., a previous or future transplant recipient), and/or undergoing chemotherapy or radiation therapy.

In some embodiments, a method of treating a disease or disorder described herein comprises administering an oligosaccharide composition provided herein to the gastrointestinal tract of a subject, and in addition or in combination administering an additional immunomodulating drug. In some embodiments, the additional immunomodulating drug is an anti-inflammatory agent. In some embodiments, the additional immunomodulating drug is a corticosteroid, e.g., budesonide, hydrocortisone, cortisone, bethamethasone, prednisone, or traiminolone. In some embodiments, the additional immunomodulating drug is self-administered, e.g., at home. In some embodiments, the additional immunomodulating drug is administered as an inhalant. Without wishing to be bound by theory, administering an oligosaccharide composition provided herein that alters one or more aspects of a subject's immune response to a respiratory illness in addition to or in combination with an additional immunomodulating drug may more effectively treat an inflammatory lung disease or disorder than either oligosaccharide composition or immunomodulating drug individually. In some embodiments, treatment with an oligosaccharide composition provided herein in addition to or in combination with an additional immunomodulating drug may reduce the severity of a symptom of an inflammatory lung disease or disorder or reduces the time to resolution of symptoms of an inflammatory lung disease or disorder, e.g., when compared to treatment with oligosaccharide composition or drug/therapy individually. In some embodiments, both the oligosaccharide composition and the additional immunomodulating drug can be self-administered, e.g., at home or at a point of care site.

In some embodiments, a method of treating a disease or disorder described herein comprises administering an oligosaccharide composition provided herein to the gastrointestinal tract of a subject, and in addition or in combination administering standard of care treatment. In some embodiments, a standard of care treatment includes bronchodilators, antibiotics and corticosteroids. In some embodiments, a standard of care treatment is triple therapy (e.g., administration of long-acting muscarinic antagonists (LAMAs), long-acting inhaled β-agonists (LABAs) and inhaled corticosteroids (ICSs)). In some embodiments, a standard of care treatment is dual bronchodilation (LAMAs and LABAs).

In some embodiments, the oligosaccharide compositions described herein are more effective in treating an inflammatory lung disease or disorder than a standard of care treatment.

In some embodiments, the oligosaccharide compositions described herein are administered in combination with a PDE4 (phosphodiesterase-4) inhibitor. In some embodiments, the oligosaccharide compositions described herein are more effective in treating an inflammatory lung disease or disorder than any single PDE4 (phosphodiesterase-4) inhibitor.

### IV. Kits

Kits also are contemplated. For example, a kit can comprise unit dosage forms of the oligosaccharide composition, and a package insert containing instructions for use of the composition in treatment. In some embodiments, the composition is provided in a dry powder format. In some embodiments, the composition is provided in solution. The kits include an oligosaccharide composition in suitable packaging for use by a subject in need thereof. Any of the compositions described herein can be packaged in the form of a kit. A kit can contain an amount of an oligosaccharide composition sufficient for an entire course of treatment, or for a portion of a course of treatment. Doses of an oligosaccharide composition can be individually packaged, or the oligosaccharide composition can be provided in bulk, or combinations thereof. Thus, in one embodiment, a kit provides, in suitable packaging, individual doses of an oligosaccharide composition that correspond to dosing points in a treatment regimen, wherein the doses are packaged in one or more packets.

Kits can further include written materials, such as instructions, expected results, testimonials, explanations, warnings, clinical data, information for health professionals, and the like. In one embodiment, the kits contain a label or other information indicating that the kit is only for use under the direction of a health professional. The container can further include scoops, syringes, bottles, cups, applicators or other measuring or serving devices.

### EXAMPLES

### Example 1. Production of the Selected Oligosaccharide Composition at 10 kg Scale from Dextrose Monohydrate, Galactose and Mannose Using a Solid Polymeric Catalyst

A “selected oligosaccharide composition” was identified in a screen of hundreds of different oligosaccharide compositions for its ability to effectively and consistently stimulate the production of short-chain fatty acids, namely butyrate, propionate, and acetate, in the microbiome residing in fecal communities of multiple healthy subjects.

A procedure was developed for the synthesis of the selected oligosaccharide composition at a 10 kilogram scale. 4.46 kg of dextrose monohydrate, 4.05 kg of galactose, 0.90 kg of mannose, and 0.90 kg (0.450 kg on a dry solid basis) of pre-conditioned solid polymeric acid catalyst (Dowex® Marathon® C resin) were added to a reaction vessel (22 L Littleford-Day horizontal plow mixer) with an attached distillation condenser unit.

The temperature controller was set to 140° C., and stirring (agitation) of the contents of the vessel at 30 RPM was initiated to promote uniform heat transfer and melting of the sugar solids, as the temperature of the syrup was brought to approximately 140° C., under ambient (atmospheric) pressure gradually over a 2.5 hour period.

The reaction mixture was maintained at temperature of approximately 140° C. for 1.5 hours (90 min), after which the heating was stopped and pre-heated water was gradually added to the reaction mixture at a rate of 60 mL/min until the temperature of the reactor contents decreased to 120° C., then at a rate of 150 mL/min until the temperature of the reactor contents decreased to 110° C., and then at a rate of 480 mL/min until the temperature of the reactor contents decreased below 100° C. and a total of 6 kg of water was added. An additional 1.75 kg of water was added to the reactor for further dilution.

The reaction mixture was drained from the vessel and the solids were removed by filtration, resulting in 15 kg of crude oligosaccharide composition product material as an aqueous solution (approximately 45 wt %).

The oligosaccharide composition was purified by flowing it through a cationic exchange resin (Dowex® Monosphere® 88H) column, two columns of decolorizing polymer resin (Dowex® OptiPore® SD-2), and an anionic exchange resin (Dowex® Monosphere® 77WBA) column. The resulting purified oligosaccharide composition had a concentration of about 35 wt % and was then concentrated to a final concentration of about 75 wt % solids by vacuum rotary evaporation.

### Example 2. Production of the Selected Oligosaccharide Composition at 10 kg Scale from Dextrose Monohydrate, Galactose and Mannose Using a Citric Acid Catalyst

A procedure was developed for the synthesis of the selected oligosaccharide composition at a 10 kilogram scale. 4.46 kg of dextrose monohydrate, 4.05 kg of galactose, 0.90 kg of mannose, 0.29 kg citric acid monohydrate acid catalyst (or 0.27 kg citric acid anhydrous) and 0.48 kg water were added to a reaction vessel (22 L Littleford-Day horizontal plow mixer). A distillation condenser unit was attached to the reactor.

The contents were agitated at approximately 30 RPM and the vessel temperature was gradually increased over a 2.5 hour period to about 139° C. at atmospheric pressure. The mixture was maintained at temperature for one and half hours. The heating was subsequently stopped and pre-heated water was gradually added to the reaction mixture at a rate of 60 mL/min until the temperature of the reactor contents decreased to 120° C., then at 150 mL/min until the temperature of the reactor contents decreased to 110° C., then at 480 mL/min until a total of 6 kg of water was added, and the temperature of the reactor contents decreased below 100° C. An additional 1.75 kg water was added to the reactor for further dilution. The reaction mixture was drained from the vessel, resulting in 15 kg of crude oligosaccharide composition product as an aqueous solution (approximately 53 wt %).

### Example 3. Production of Oligosaccharide Composition at 100 g Scale from Dextrose Monohydrate, Galactose and Mannose Using a Solid Polymeric Catalyst

A procedure was developed for the synthesis of the selected oligosaccharide composition at a 100 gram scale. 45 g of dextrose monohydrate, 45 g of galactose, 10 g of mannose were added to a reaction vessel (1 L three-neck round-bottom flask). The reaction vessel was equipped with a heating mantle configured with an overhead stirrer. A probe thermocouple was disposed in the vessel through a septum, such that the probe tip sat above the stir blade and not in contact with the walls of the reaction vessel.

The procedure also used an oligomerization catalyst (Dowex Marathon C) (3-5% w/w) and de-ionized water for quenching. In some cases, the catalyst was handled in wet form, e.g., at a nominal moisture content of 45-50 wt % H₂O. The exact catalyst moisture content was generally determined on a per-experiment basis using, for example, using a moisture analyzing balance (e.g., Mettler-Toledo MJ-33).

The temperature controller was set to a target temperature (100 to 160° C.), and stirring of the contents of the vessel was initiated to promote uniform heat transfer and melting of the sugar solids, as the temperature of the syrup was brought to the target temperature, under ambient (atmospheric) pressure.

Upon addition of the catalyst, the reaction was maintained at the target temperature under continuous mixing for about 4 hours, determined by following the reaction by HPLC. Next, the heat was turned off while maintaining constant stirring.

The reaction was then quenched by slowly adding approximately 60 mL of hot (˜80° C.) deionized (DI) water to dilute and cool the product mixture, to target a final concentration of 70 wt % dissolved solids. Generally, the water addition rate was performed to control the mixture viscosity as the oligosaccharide composition was cooled and diluted.

Following dilution, the oligosaccharide composition was cooled to approximately 60° C. The catalyst was then removed by vacuum filtration through a 100 micron mesh screen or fritted-glass filter, to obtain the final oligosaccharide composition at around 40° Bx.

### Example 4. De-Monomerization Procedure

Individual batches of oligosaccharide composition, as produced in Examples 1-3 were concentrated on a rotatory evaporator to approximately 50 Brix as measured by a Brix refractometer. The resulting syrup (200 mg) was loaded onto a Teledyne ISCO RediSep Rf Gold Amine column (11 grams stationary phase) using a luer-tip syringe. Other similar columns such as the Biotage SNAP KP-NH Cartridges may also be used. The sample was purified on a Biotage Isolera equipped with an ELSD detector using a 20/80 to 50/50 (v/v) deionized water/ACN mobile phase gradient over 55 column volumes. Other flash chromatography systems such as the Teledyne ISCO Rf may also be used. The flow rate was set in accordance with the manufacturer's specifications for the column and system. After the monomer fraction completely eluted at ˜20 column volumes, the mobile phase was set to 100% water until the remainder of the oligosaccharide composition eluted and was collected. The non-monomer containing fractions were concentrated by rotary evaporation to afford the de-monomerized product.

### Example 5. Collection of Fecal Samples

Fecal samples were collected by providing subjects with the Fisherbrand Commode Specimen Collection System (Fisher Scientific) and associated instructions for use. Collected samples were stored with ice packs or at −80° C. until processing (Mclnnes & Cutting, Manual of Procedures for Human Microbiome Project: Core Microbiome Sampling Protocol A, 2010, hmpdacc.org/doc/HMP_Clinical_Protocol.pdf). Alternative collection devices may also be used. For example, samples may be collected into the Faeces Tube 54×28 mm (Sarstedt A G, 25 ml SC Feces Container w/Scoop), Globe Scientific Screw Cap Container with Spoon (Fisher Scientific) or the OMNIgene-GUT collection system (DNA Genotek, Inc.), which stabilizes microbial DNA for downstream nucleic acid extraction and analysis. Aliquots of fecal samples were stored at −20° C. and −80° C. following standard protocols known to one skilled in the art.

### Example 6. The Selected Oligosaccharide Composition Increases SCFA Production in Fecal Suspensions from Healthy Subjects

The ability of the selected oligosaccharide composition comprised of a plurality of oligosaccharides selected from Formula (I), Formula (II), and Formula (III) as produced by a similar process as described in Examples 1-3 to increase the production of short-chain fatty acids (SCFAs) in fecal suspensions of healthy subjects was assessed.

Fecal samples were collected from healthy subjects and stored at −80° C. All fecal sample preparation was performed in a Coy anaerobic chamber. Fecal samples were thawed, 20% (w/v) fecal suspensions were prepared in phosphate-buffered saline (PBS) supplemented with 15% glycerol, dispensed into 1 ml aliquots, and stored at −80° C. 1 ml aliquots of 20% (w/v) fecal suspensions were thawed and sedimented at sedimented by centrifugation 2,000×g for 5 minutes. The supernatants were aspirated and discarded. The fecal pellets were resuspended in 1 ml of PBS. 1% (w/v) fecal suspensions were prepared by adding 12.5 ml of 20% fecal suspension to 237.5 ml of Clostridium minimal medium supplemented with 0.1% (w/v) trypticase peptone and 0.75 mM urea. 315 μl of the 1% fecal suspensions were dispensed into the wells of 96-well deep well plates containing 35 μl of water (negative control) or 5% (w/v) oligosaccharide solutions (final oligosaccharide concentration of 0.5%). Three replicates of each sample were prepared. Fecal microbial cultures were incubated anaerobically at 37° C. for 45 hours.

After the 45-hour incubation period, the 96-well deep well plates containing the fecal microbial cultures were removed from the Coy anaerobic chamber and kept on ice. The plates were sedimented by centrifugation (3,000×g) for 10 minutes at 4° C. Fecal microbiota culture supernatants were collected and stored at −80° C. Supernatant samples were thawed and analyzed by gas chromatography with a flame ionization detector (GC-FID) to quantify the short-chain fatty acids (acetate, propionate, and butyrate). Normalized SCFA concentrations were calculated by subtracting the value with the negative control (water without oligosaccharide) from oligosaccharide composition treatment. This normalized value represents the concentration of SCFA derived from fermentation of the selected oligosaccharide composition. The median normalized SCFA concentrations were determined for each set of three sample replicates.

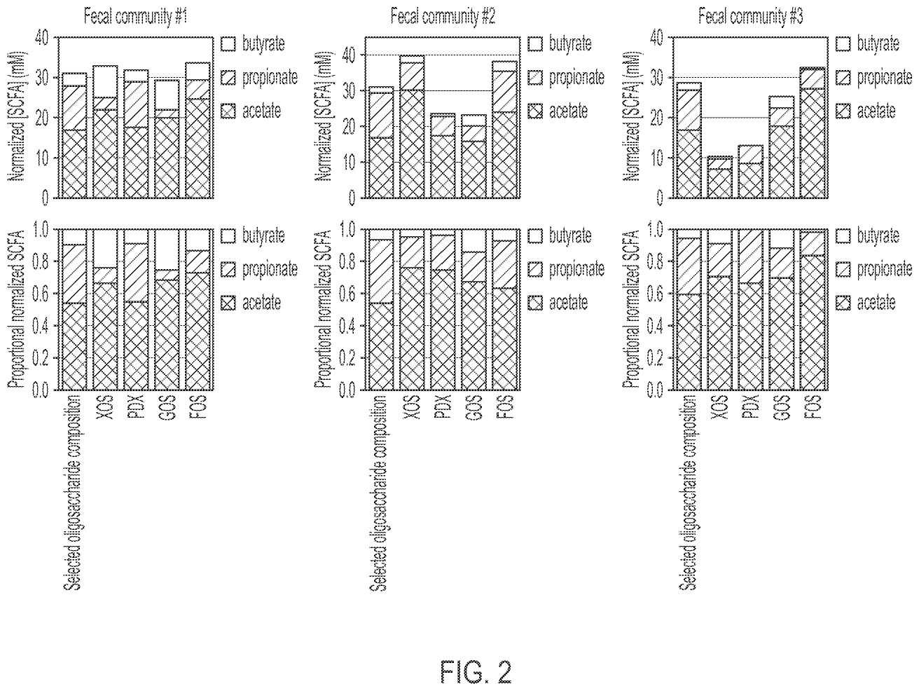

Three different fecal microbiota from healthy subjects were incubated with 0.5% (w/v) of the selected oligosaccharide composition or one of four different commercial oligosaccharides. Following the incubation, the concentration of three different SCFAs (acetate, propionate, and butyrate) were measured in the fecal microbiota culture supernatants by gas chromatography-flame ionization detection (GC-FID). Incubation with the selected oligosaccharide composition resulted in the production of between 28 and 32 mM total SCFA across the three fecal communities (FIG. 2). In addition to consistent total SCFA production across the fecal communities, the selected oligosaccharide composition also produced consistent proportions of acetate, propionate, and butyrate, with 54-60% acetate, 34-40% propionate, and 6-10% butyrate. Conversely, the commercial oligosaccharides (xylo-oligosaccharide (XOS), polydextrose (PDX), and galacto-oligosaccharide (GOS)) produced less consistent total SCFA concentrations than the selected oligosaccharide composition. Although total SCFA production with fructo-oligosaccharide (FOS) was consistent, the relative proportions of individual SCFAs (acetate, propionate, butyrate) were less consistent. This inconsistency is most apparent with the butyrate proportions. FOS produced 13% butyrate in first fecal community, 7% in the second, and only 2% in the third. These data collectively demonstrate that the selected oligosaccharide composition robustly and consistently increases total SCFA while maintaining a consistent ratio of acetate:propionate:butyrate.

Additionally, seven different fecal microbiota from healthy subjects were incubated with the selected oligosaccharide composition. The selected oligosaccharide composition increased the production of SCFA between 4-fold and 6-fold compared to the negative control across the seven fecal communities (FIGS. 1A-1B). These data further demonstrate that the selected oligosaccharide composition robustly and consistently increases total SCFA while maintaining a consistent ratio of acetate:propionate:butyrate across seven different fecal communities.

### Example 7. Clinical Trial to Assess Ability of the Selected Oligosaccharide Composition to Treat Respiratory Infections and Impact the Gut-Lung Axis

The ability of the selected oligosaccharide composition comprised of a plurality of oligosaccharides selected from Formula (I), Formula (II), and Formula (III) as produced by a similar process as described in Examples 1-3 was assessed for its ability to treat and prevent respiratory infections (e.g., COVID-19 infections).

The study (a non-IND clinical study conducted under regulations supporting research with food, evaluating safety, tolerability and potential markers of human effect) was a randomized, controlled, multi-site, open label clinical study to assess the selected oligosaccharide composition on safety as well as measures of signs, symptoms, healthcare utilization (including hospitalizations), laboratory/biochemical indices and quality-of-life measures in outpatients who have tested positive with COVID-19 who have mild-to-moderate disease, and have been advised to manage their disease at home with supportive self-care (SSC) under quarantine protocols set forth by the United States Center for Disease Control in 2020.

350 patients enrolled with mild to moderate COVID-19 symptoms were randomized (1:1) to receive either (A) SSC and the selected oligosaccharide composition or (B) remain on SSC alone. The randomization was stratified by site/center, age subgroup (≥18 to <45, ≥45 to <65, ≥65), and comorbidity status (e.g., concurrent medical conditions). The study protocols and results are as reported in Haran, J. P. et. al., “Targeting the Microbiome With KB109 in Outpatients with Mild to Moderate COVID-19 Reduced Medically Attended Acute Care Visits and Improved Symptom Duration in Patients With Comorbidities,” medRxiv, published online on Mar. 29, 2021; the entire contents of which are incorporated herein by reference.

Patients self-recorded symptoms associated with lung disease and inflammation (cough, fever, nasal congestion, gastrointestinal symptoms, fatigue, anosmia, ageusia, diarrhea, shortness of breath, chest tightness, and headache) and scored them using a rating of 0-3 where 0 means that the symptom is absent, 1 means that the symptom is mild (present but easily tolerated), 2 means that the symptom is moderately severe (bothersome but tolerable), and 3 means that the symptom is very severe (hard to tolerate; interferes considerably with daily activity).

Patients having a positive test result for COVID-19 were randomized into one of the two treatment groups (SSC and the selected oligosaccharide composition; or SSC alone), and an at home Study Kit (KaSK) was provided to the patient. The KaSK included study product and dosing instructions (as applicable), a thermometer, a pulse oximeter, telemedicine contact information, and return shipping materials. Patients continued to record COVID-19-related symptoms using the same scoring rubric until the KaSK was delivered.

The Intake Period (Days 1-14) began the morning after receipt of the KaSK. On each of days 1 and 2, the patients consumed 18 grams of the selected oligosaccharide composition. On each of days 3 and 4, the patients consumed 36 grams of the selected oligosaccharide composition. On each of days 5-14, the patients consumed 72 grams of the selected oligosaccharide composition.

During the Intake Period, for all patients, one or more of blood, nasal and oropharyngeal swabs were collected by home nursing providers or remotely, as feasible, and as close to the beginning (Day 1) and end (Day 14) of the Intake Period for analysis of laboratory, biochemical and serological measures.

On Day 14, all patients underwent another telemedicine visit where an abbreviated physical examination is conducted, an assessment of safety, and an evaluation of whether follow-up treatment was recommended due to a deterioration of COVID-19 symptoms. Patients in the SSC and selected oligosaccharide composition group stopped taking the selected oligosaccharide composition on Day 14.

On Day 15, all patients entered the Follow-up Period (Days 15 to 35) where COVID-19 signs, symptoms, quality of life and Health Care Utilization indices were collected weekly in the secure online Study Portal. Blood, nasal and oropharyngeal swabs were collected by home nursing providers or remotely, as feasible, and as close to the end of the Follow-up Period (Day 35) for analysis of laboratory, biochemical and serological measures.

On Day 35, all patients underwent another telemedicine visit where an abbreviated physical examination was conducted, an assessment of safety, and an evaluation of whether follow-up treatment is recommended.

A full analysis was performed on the completed 350 person study data set. 350 subjects were randomized to either SSC alone (181 subjects) or SSC+selected oligosaccharide (169 subjects).

The demographics of both arms were generally well balanced. The majority of subjects in the ≥18 to <45 years (234 subjects, 66.9%) age group, followed by the ≥45 to <65 years (99 subjects, 28.3%) age group, and a small ≥65 years (17 subjects, 5%) age group. There were more females (207 subjects, 59.1%) than males (143 subjects, 40.9%). The majority of subjects were white (318 subjects, 90.9%). Other races included black/African American (26 subjects, 7.4%), Asian (4 subjects, 1.1%) and other (2 subjects, 0.6%).

BMI ranged from 16.4 to 63.9 (with overweight defined as 25-29.9 and obese defined as BMI of 30 or greater) with a mean of 28.74 (+/−6.826) in the SSC+selected oligosaccharide arm and a mean of 28.99 (+/−6.167) in the SSC alone arm, and an overall mean of 28.86 (+/−6.49).

In the SSC+selected oligosaccharide arm, 69 subjects (40.8%) had at least one comorbidity, while 66 subjects (36.5%) had at least one comorbidity in the SSC alone arm.

The level of C-reactive protein (CRP), a biomarker of systemic inflammation in the bloodstream, was monitored for study participants at days 0, 14, and 35. Participants who had one or more comorbidities might be more likely to suffer from chronic inflammation, independent of their symptoms, and it was hypothesized that they could experience a decrease in inflammation from treatment with an oligosaccharide composition that promotes microbial populations that decrease inflammation in the gastrointestinal tract.

Several biomarkers of inflammation were measured, including C-reactive protein. No significant differences were measured for C-reactive protein for subjects without comorbidity for SSC alone (a) versus SSC+selected oligosaccharide (b): mean values at baseline were 3.128 mg/L for (a) versus 3.018 mg/L for (b), and at 35 days, 2.215 mg/L for (a) versus 2.470 for (b). A trend toward reduction in C-reactive protein was found in subjects with at least one comorbidity in the SSC+selected oligosaccharide arm: at baseline, the mean value was 9.908 mg/L (vs. 5.254 mg/L in the SSC alone arm), and at 35 days, C-reactive protein dropped to 3.876 mg/L in the SSC+selected oligosaccharide arm with subjects with comorbidity (vs. 4.082 mg/L in the SSC alone arm), although there was high variability between subjects.

While not statistically significant, this is consistent with the idea that the modulation of the microbiome induced by the oligosaccharide composition promotes an attenuation of one or more aspects of a subject's immune response, potentially attenuating undesirable excessive inflammatory responses to viral illness.

The decline in symptoms associated with lung disease and inflammation over time as the infection progresses was assessed as part of the safety assessment in the full subject pool and was measured by analysing 13 overall symptoms (cough, fever, nasal congestion, gastrointestinal symptoms, fatigue, anosmia, ageusia, diarrhea, shortness of breath, chest tightness, and headache) in the overall participant pool, participants having at least one comorbidity, and participants not having a comorbidity. Patients were treated with SSC alone (Arm 1) or SSC and the selected oligosaccharide composition (Arm 2). The data was consistent with the interim analysis described above. The median time to resolution of symptoms in patients was measured:
21.0 (95% CI: 16.0, 28.0) days for patients with at least one comorbidity treated with SSC and oligosaccharide versus 30.0 (95% CI: 20.0, 32.0) days for patients with at least one comorbidity on SSC alone;
18.0 (95% CI: 14.0, 23.0) days for patients without comorbidities treated with SSC and oligosaccharide versus 21.0 (95% CI: 15.0, 29.0) days for patients without comorbidities on SSC alone;
19.0 (95% CI: 16.0, 23.0) days for the overall patient population treated with SSC and oligosaccharide versus 22.0 (95% CI: 19.0, 30.0) days for the overall patient population on SSC alone.
   The data confirmed the oligosaccharide composition was generally safe and tolerable. The data further suggest that the selected oligosaccharide may be useful in reducing the time to resolution of the 13 symptoms in patients generally, regardless of whether the patient also exhibits a comorbidity. In addition, the data suggests patients displaying one or more comorbidity receive additional benefit, resolving symptoms more than a week faster when treated with SSC and oligosaccharide composition compared to SSC alone.

Levels of Tumor necrosis factor-alpha (TNF-alpha) and/or Interferon gamma (IFN-gamma) in plasma of subjects having high baseline levels of TNF-alpha and/or IFN-gamma were further analyzed throughout the treatment period (at time of screening, on Day 14 and on Day 35). See, Table 8. These subjects had higher levels of TNF-alpha and/or IFN-gamma than the median subject in the Example.

TABLE 8
Average plasma concentrations of TNF-alpha and IFN-gamma in subjects

| | | Screening | Day 14 | Day 35 |
|---|---|---|---|---|
| IFN-gamma | SSC + selected | 35 | 24 | 27 |
| | oligosaccharide | pg/mL | pg/mL | pg/mL |
| | composition | | | |
| IFN-gamma | SSC Alone | 24 | 20 | 15 |
| | | pg/mL | pg/mL | pg/mL |
| TNF-alpha | SSC + + selected | 31 | 23 | 23 |
| | oligosaccharide | pg/mL | pg/mL | pg/mL |
| | composition | | | |
| TNF-alpha | SSC Alone | 28 | 24 | 19 |
| | | pg/mL | pg/mL | pg/mL |

TNF-alpha and IFN-gamma concentrations were measured using the MSD V-PLEX Proinflammatory Panel 1 Human Kit; and for each biomarker, the distribution of within-subject log 2 fold changes (log 2FC) from screening at Days 14 and 35 were plotted using box-whisker plots overlayed with dots representing individual subjects. The difference of the changes between subjects treated with SSC+oligosaccharide composition compared to subjects treated with SSC alone were tested using Wilcoxon rank sum test.

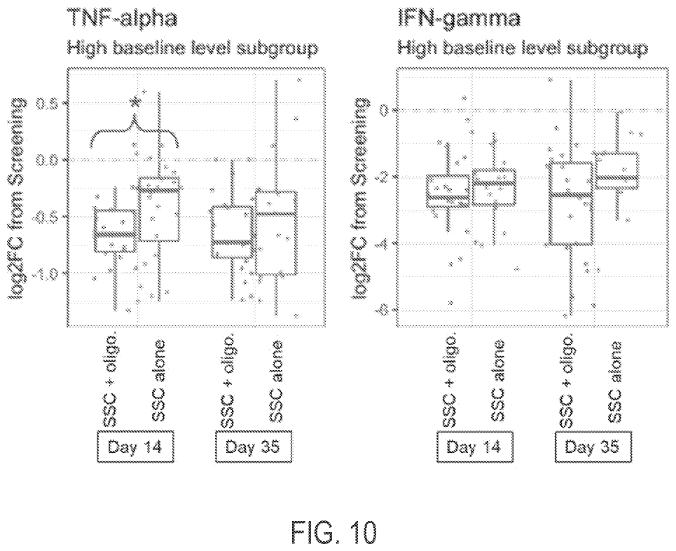

Subjects treated with SSC and the selected oligosaccharide composition had an average reduction in TNF-alpha of 8 μg/mL and an average reduction in IFN-gamma of 11 μg/mL, at Day 14 relative to screening. These reductions were larger than shown in subjects treated with SSC alone. See, Table 8 and FIG. 10.

These data demonstrate that the selected oligosaccharide composition functions to systemically reduce pro-inflammatory biomarkers (TNF-alpha and IFN-gamma). More specifically, these data demonstrate that the selected oligosaccharide composition accelerates reduction of TNF-alpha and sustained reduction of IFN-gamma in patients with high levels of pro-inflammatory biomarkers.

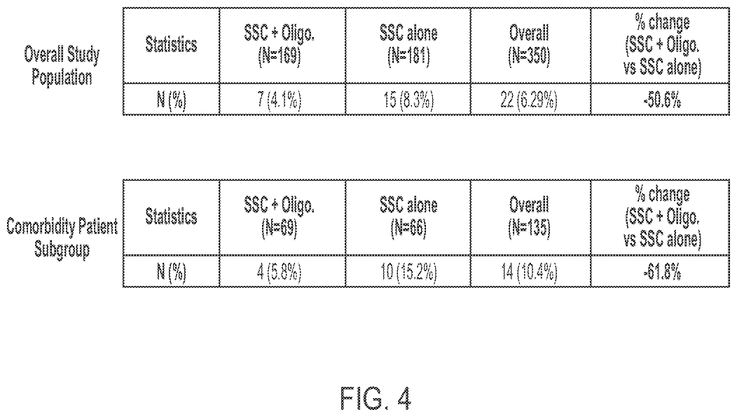

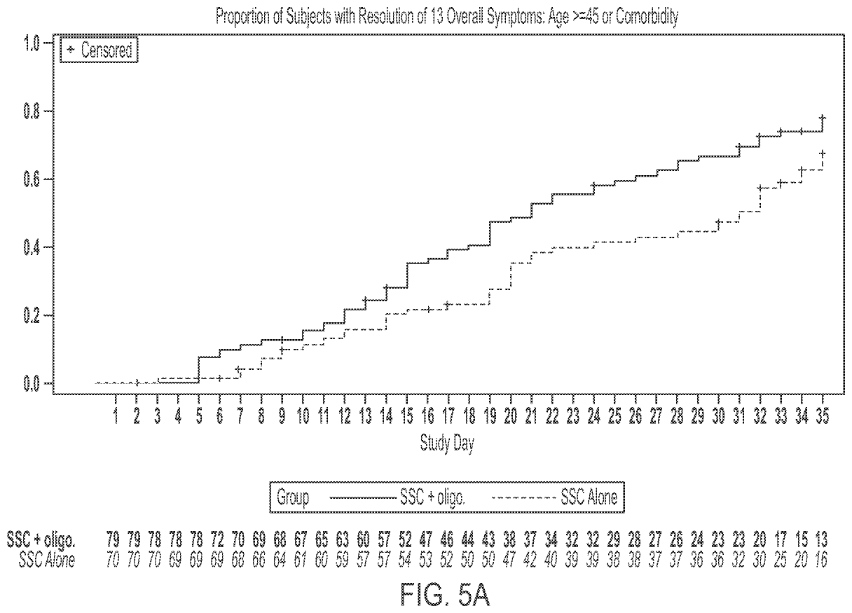

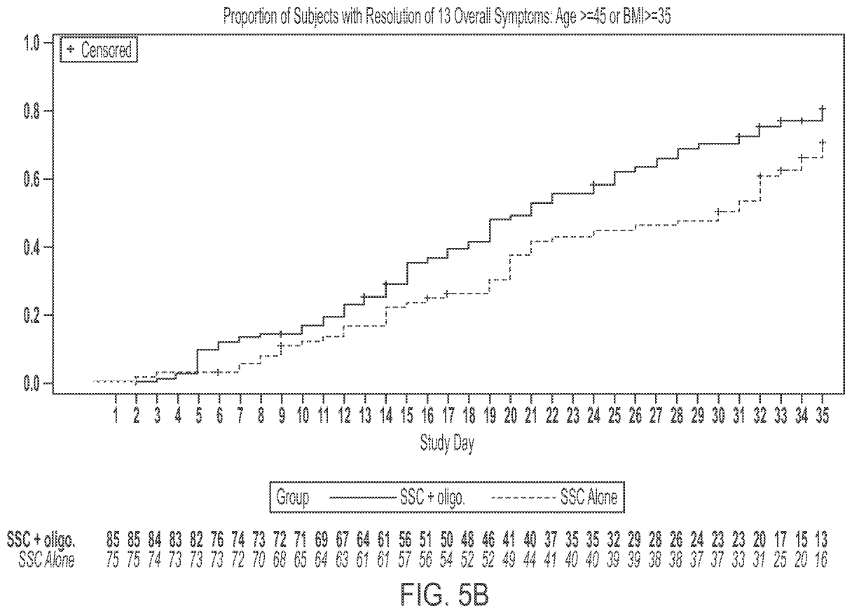

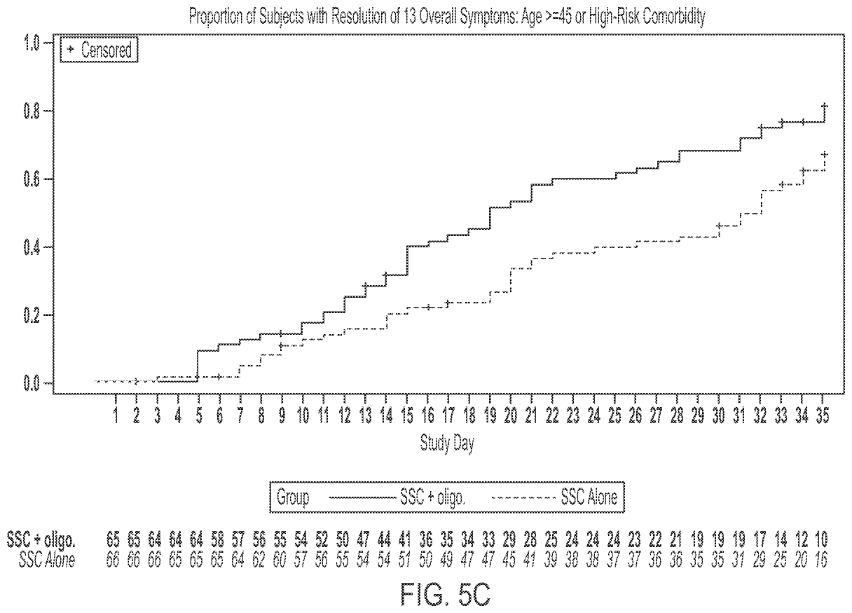

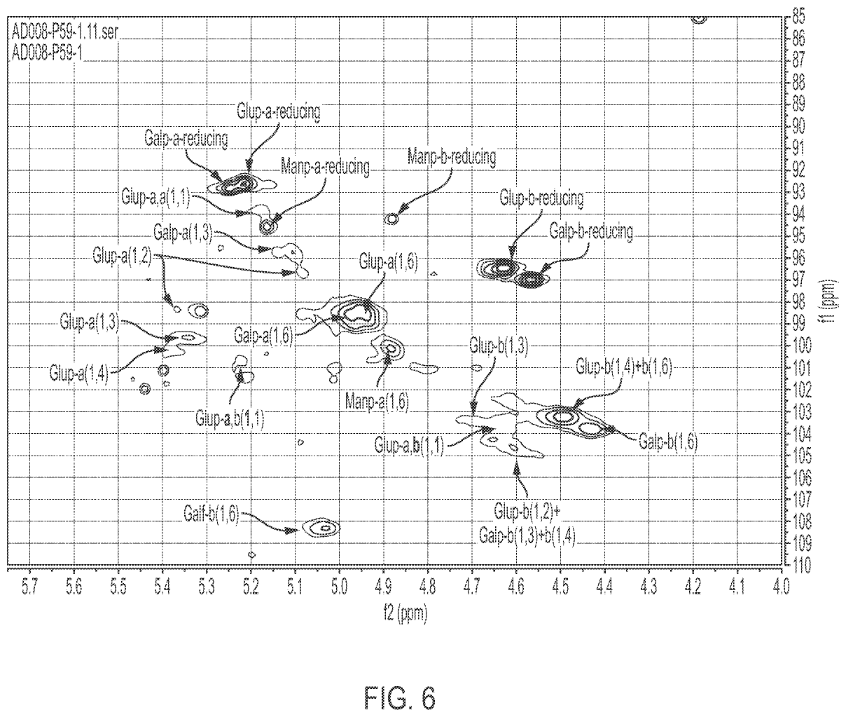

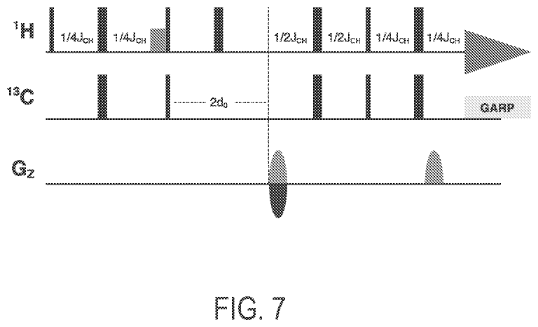

The number of patients utilizing healthcare via a hospitalization, emergency room visit, or urgent care visit was assessed at the study's completion (FIG. 4). Subjects in the overall population treated with SSC and oligosaccharide composition had a healthcare utilization rate more than 50% lower than subjects treated with SSC alone. Subjects having one or more comorbidity treated with SSC and oligosaccharide composition had a healthcare utilization rate more than 61% lower than similar subjects treated with SSC alone. These data suggest that administration of the oligosaccharide composition may have healthcare system-wide benefits in addition to patient or patient population level benefits, and may be useful for decreasing the burden that lung diseases or infections such as COVID-19 place on healthcare systems.

Patients having one or more additional risk factor have been shown to be at a higher risk of mild to moderate COVID-19 progressing to severe COVID-19, as well as for experiencing more severe long side effects. A series of subgroups of participants were evaluated—as part of the safety assessment—for their time to resolution of symptoms: patients with an age-related risk factor (e.g., at least 45 years old); patients with one or more comorbidity; patients with one or more high-risk comorbidity (e.g., chronic lung disease (e.g., asthma, emphysema, or COPD), diabetes mellitus, cardiovascular disease, hypertension, chronic renal disease, or cancer); and obese patients (e.g., having a BMI >35). Sec, FIGS. 4A-4C. The data show that the selected oligosaccharide composition reduces the time to resolution of symptoms in patients at least 45 years old; in patients with at least one comorbidity; or being overweight or obese (e.g., a BMI of at least 30 or at least 35). This suggests that the selected oligosaccharide composition reduced the time to resolution of symptoms of an exemplary viral respiratory illness, COVID-19, that impacts the lung in the overall study patient population, irrespective of other health conditions. The study suggests that changes in a subject's microbiome promoted by the selected oligosaccharide composition can reduce systemic inflammation, e.g., a biomarker of systemic inflammation (see, CRP; and TNF-alpha and IFN-gamma, as shown in FIG. 10). The study shows that the selected oligosaccharide composition reduced the time to resolution of symptoms in the lungs of patients with at least one comorbidity, an age-related risk factor, who are overweight or obese, or who have two or more of these criteria. The time to resolution of symptoms was further reduced in these high-risk subjects relative to the overall study patient population. The study shows that, in addition to hastening the recovery of patients, patients treated with the selected oligosaccharide composition showed a decrease in healthcare utilization, showing the potential of treatment with the selected oligosaccharide composition to case the burdens not just on patients themselves, but the overburdened healthcare systems serving them.

### Example 8. Determination of Glycosidic Bond Distribution Using Permethylation Analysis

A determination of glycosidic bond distribution of samples of the selected oligosaccharide composition, as produced by a process as described in Example 1 (Marathon C catalyst), was performed using permethylation analysis, according to the protocol described below. Samples were demonomerized prior to permethylation analysis.

Reagents used were methanol, acetic acid, sodium borodeuteride, sodium carbonate, dichioromethane, isopropanol, trifluoroacetic acid (TFA), and acetic anhydride. Equipment included a heating block, drying apparatus, gas chromatograph equipped for capillary columns and with a RID/MSD detector, and a 30 meter RTX®-2330 (RESTEK). All derivation procedures were done in a hood.

### Preparation of Alditol Acetates

### A. Standard Preparation

1 mg/mL solutions of the following standard analytes were prepared: arabinose, rhamnose, fucose, xylose, mannose, galactose, glucose, and inositol. The standard was prepared by mixing 50 μL of each of arabinose, xylose, fucose, glucose, mannose, and galactose with 20 μL of inositol in a vial. The standard was subsequently lyophilized.

### B. Sample Preparation

Each sample was prepared by mixing 100-500 μg of the selected oligosaccharide composition (as weighed on an analytical balance) with 20 μg (20 μL) of inositol in a vial.

### C. Hydrolysis

200 μL of 2 M tifluoroacetic acid (TFA) was added to the sample(s). The vial containing the sample was capped tightly and incubated on a heating block for 2 hours at 121° C. After 2 hours, the sample was removed from the heating block and allowed to cool to room temperature. The sample was then dried down with N₂/air. 200 μL of IPA (isopropanol) was added and dried down again with N₂/air. This hydrolysis step (addition of TFA for two hours at 121° C.; washing with isopropanol) was repeated twice.

The standard was similarly subjected to hydrolysis using TFA, as described for the sample.

### D. Reduction and Acetylation

10 mg/mL solution of sodium borodeuteride was prepared in 1 M ammonium hydroxide. 200 μL of this solution was added to the sample. The sample was then incubated at room temperature for at least one hour or overnight. After incubation with sodium borodeuteride solution, 5 drops of glacial acetic acid were added to the sample, followed by 5 drops of methanol. The sample was then dried down. 500 μL of 9:1 MeOH:HOAc was added to the sample and subsequently dried down (twice repeated). 500 μL MeOH was then added to the sample and subsequently dried down (once repeated). This produced a crusty white residue on the side of the sample vial.

250 μL acetic anhydride was then added to the sample vial and the sample was vortexed to dissolve. 230 μL concentrated TFA was added to the sample and the sample was incubated at 50° C. for 20 minutes. The sample was removed from the heat and allowed to cool to room temperature. Approximately 1 mL isopropanol was added and the sample was dried down. Then, approximately 200 μL isopropanol was added and the sample was dried down again. Approximately 1 mL of 0.2M sodium carbonate was then added to the sample and it was mixed gently. Approximately 2 mL dichloromethane was finally added to the sample, after which it was vortexed and centrifuged briefly. The aqueous top layer was discarded. 1 mL water was added and the sample was vortexed and centrifuged briefly. This step was repeated before the organic layer (bottom) was removed and transferred to another vial. The sample was concentrated using N₂/air to a final volume of about 100 μL. 1 μL of final sample was then injected on GC-MS.

The GC temperature program SP2330 was utilized for GC-MS analysis. The initial temperature was 80° C. and the initial time was 2.0 minutes. The first ramp was at a rate of 30° C./min with a final temperature of 170° C. and a final time of 0.0 minutes. The second ramp was at a rate of 4° C./min with a final temperature of 240° C. and a final time of 20.0 minutes.

### Glycosyl-Linkage Analysis of Poly- and Oligosaccharides by Hakomori Methylation

A. Preparation of NaOH base

In a glass screw top tube, 100 μL of a 50/50 NaOH solution and 200 μL of dry MeOH were combined. Plastic pipets were used for the NaOH and glass pipets were used for the MeOH. The solution was vortexed briefly, approximately 4 mL dry DMSO was added, and the solution was vortexed again. The tube was centrifuged to concentrate the solution and the DMSO and salts were pipetted off from the pellet. The previous two steps were repeated about four times in order to remove all the water from the pellet. All white reside was removed from the sides of the tube. Once all the residue was removed and the pellet was clear, about 1 mL dry DMSO was added and the solution was vortexed. The base was then ready to use. The base was prepared fresh each time it was needed.

### B. Permethylation

Each sample was prepared by mixing 600-1000 μg of the selected oligosaccharide composition (as weighed on an analytical balance) with 200 μL DMSO. The sample was stirred overnight until the oligosaccharide composition dissolved.

An equal amount of NaOH base (400 μL) was added to the sample, after which the sample was placed back on the stirrer and mixed well for 10 minutes. 100 μL of iodomethane (CH₃I) was added to the sample. The sample was mixed on the stirrer for 20 minutes, and then the previous steps (addition of NaOH base and iodomethane) were repeated.

Approximately 2 mL ultrapure water was added to the sample and the sample was mixed well, such that it turned cloudy. The tip of a pipette was placed into the sample solution at the bottom of the tube and CH₃I was bubbled off with a very low flow of air. The sample became clear as the CH₃I was bubbled off. The pipette was moved around the solution to make certain that all the CH₃I was gone. Approximately 2 mL methylene chloride was then added and the solution was mixed well by vortex for 30 seconds. The sample was then centrifuged and the top aqueous layer was removed. Approximately 2 mL of water were added and the sample was mixed, then briefly centrifuged, then the top aqueous layer was removed. The additions of methylene chloride and water were repeated. The organic bottom layer was removed and transferred into another tube and dried down using N₂. The analysis was continued with Alditol Acetates.

### C. Hydrolysis

200 μL of 2 M tifluoroacetic acid (TFA) was added to the sample(s). The vial containing the sample was capped tightly and incubated on a heating block for 2 hours at 121° C. After 2 hours, the sample was removed from the heating block and allowed to cool to room temperature. The sample was then dried down with No/air. 200 μL of IPA (isopropanol) was added and dried down again with N₂/air. This hydrolysis step (addition of TFA for two hours at 121° C.; washing with isopropanol) was repeated twice.

### D. Reduction and Acetylation

10 mg/mL solution of sodium borodeuteride was prepared in 1 M ammonium hydroxide. 200 μL of this solution was added to the sample. The sample was then incubated at room temperature for at least one hour or overnight. After incubation with sodium borodeuteride solution, 5 drops of glacial acetic acid were added to the sample, followed by 5 drops of methanol. The sample was then dried down. 500 μL of 9:1 MeOH:HOAc was added to the sample and subsequently dried down (twice repeated). 500 μL MeOH was then added to the sample and subsequently dried down (once repeated). This produced a crusty white residue on the side of the sample vial.

250 μL acetic anhydride was then added to the sample vial and the sample was vortexed to dissolve. 230 μL concentrated TFA was added to the sample and the sample was incubated at 50° C. for 20 minutes. The sample was removed from the heat and allowed to cool to room temperature. Approximately 1 mL isopropanol was added and the sample was dried down. Then, approximately 200 μL isopropanol was added and the sample was dried down again. Approximately 1 mL of 0.2M sodium carbonate was then added to the sample and it was mixed gently. Approximately 2 mL dichloromethane was finally added to the sample, after which it was vortexed and centrifuged briefly. The aqueous top layer was discarded. 1 mL water was added and the sample was vortexed and centrifuged briefly. This step was repeated before the organic layer (bottom) was removed and transferred to another vial. The sample was concentrated using N₂/air to a final volume of about 100 μL. 1 μL of final sample was then injected on GC-MS.

The GC temperature program SP2330 was utilized for GC-MS analysis. The initial temperature was 80° C. and the initial time was 2.0 minutes. The first ramp was at a rate of 30° C./min with a final temperature of 170° C. and a final time of 0.0 minutes. The second ramp was at a rate of 4° C./min with a final temperature of 240° C. and a final time of 20.0 minutes.

### Results

Permethylation data was collected using the methods described above for four batches of de-monomerized oligosaccharide composition produced by a process as described in Example 1 (Marathon C catalyst). Each batch was analyzed in duplicate. Averaged data relating to the radicals present in these ten batches of de-monomerized oligosaccharide composition are provided below:

| | Mean | | Mean |
|---|---|---|---|
| | mol % +3 | Mean | mol % −3 |
| Radicals | STD | mol % | STD |
| t-manopyranose | 4.10% | 3.56% | 3.02% |
| t-glucopyranose | 16.33% | 13.89% | 11.44% |
| t-galactofuranose | 7.78% | 4.52% | 1.26% |
| t-glucofuranose | 1.38% | 0.64% | 0.00% |
| t-galactopyranose | 12.48% | 10.38% | 8.29% |
| 3-glucopyranose | 4.88% | 3.95% | 3.02% |
| 2-manopyranose and/or | 1.94% | 1.57% | 1.20% |
| 3-manopyranose | | | |
| 2-glucopyranose | 3.22% | 2.83% | 2.44% |
| 2-galactofuranose and/or | 2.32% | 1.62% | 0.93% |
| 2-glucofuranose | | | |
| 3-galactopyranose | 3.92% | 3.43% | 2.94% |
| 4-manopyranose and/or | 2.93% | 2.34% | 1.75% |
| 5-manofuranose and/or | | | |
| 3-galactofuranose | | | |
| 6-manopyranose | 2.87% | 2.44% | 2.01% |
| 2-galactopyranose | 2.71% | 2.28% | 1.85% |
| 6-glucopyranose | 10.78% | 9.22% | 7.66% |
| 4-galactopyranose and/or | 3.80% | 3.22% | 2.65% |
| 5-galactofuranose | | | |
| 4-glucopyranose and/or | | | |
| 5-glucofuranose and/or | 4.25% | 3.66% | 3.06% |
| 6-manofuranose | | | |
| 6-glucofuranose | 1.55% | 0.81% | 0.08% |
| 6-galactofuranose | 4.96% | 3.19% | 1.42% |
| 6-galactopyranose | 9.06% | 7.44% | 5.81% |
| 3,4-galactopyranose and/or | | | |
| 3,5-galactofuranose and/or | 1.42% | 1.16% | 0.90% |
| 2,3-galactopyranose | | | |
| 3,4-glucopyranose and/or | 1.04% | 0.43% | 0.00% |
| 3,5-glucofuranose | | | |
| 2,4-glucopyranose and/or | | 1.16% | |
| 2,5-glucofuranose and/or | | | |
| 2,4-galactopyranose and/or | 1.39% | | 0.92% |
| 2,5-galactofuranose | | | |
| 4,6-manopyranose and/or | 0.69% | 0.59% | 0.49% |
| 5,6-manofuranose | | | |
| 3,6-manofuranose | 0.11% | 0.02% | 0.00% |
| 3,6-glucopyranose | 2.80% | 2.10% | 1.40% |
| 3,6-manopyranose and/or | 0.67% | 0.53% | 0.39% |
| 2,6-manofuranose | | | |
| 2,6-manopyranose | 0.54% | 0.41% | 0.28% |
| 3,6-glucofuranose | 0.39% | 0.27% | 0.16% |
| 2,6-glucopyranose and/or | 3.58% | 2.33% | |
| 4,6-glucopyranose and/or | | | 1.08% |
| 5,6-glucofuranose | | | |
| 3,6-galactofuranose | 1.37% | 1.15% | 0.93% |
| 4,6-galactopyranose and/or | 2.86% | 2.48% | 2.11% |
| 5,6-galactofuranose | | | |
| 3,6-galactopyranose and/or | 2.98% | 2.28% | 1.58% |
| 2,6-galactofuranose | | | |
| 2,6-galactopyranose | 1.62% | 1.15% | 0.68% |
| 3,4,6-manopyranose and/or | 0.30% | 0.07% | 0.00% |
| 3,5,6-manofuranose and/or | | | |
| 2,3,6-manofuranose | | | |
| 3,4,6-galactopyranose and/or | 1.11% | 0.82% | 0.53% |
| 3,5,6-galactofuranose and/or | | | |
| 2,3,6-galactofuranose | | | |
| 3,4,6-glucopyranose and/or | 0.47% | 0.35% | 0.22% |
| 3,5,6-glucofuranose | | | |
| 2,3,6-manopyranose and/or | 0.49% | 0.17% | 0.00% |
| 2,4,6-manopyranose and/or | | | |
| 2,5,6-manofuranose | | | |
| 2,4,6-glucopyranose and/or | 1.36% | 0.56% | 0.00% |
| 2,5,6-glucofuranose | | | |
| 2,3,6-galactopyranose and/or | | | |
| 2,4,6-galactopyranose and/or | 0.91% | 0.66% | 0.41% |
| 2,5,6-galactofuranose | | | |
| 2,3,6-glucopyranose | 0.48% | 0.31% | 0.13% |

Permethylation data was collected using the methods described above for five batches of de-monomerized oligosaccharide composition produced by the process described in Example 2 (citric acid catalyst). Each batch was analyzed in duplicate. Averaged data relating to the radicals present in these ten batches of de-monomerized oligosaccharide composition are provided below:

| | Mean | | Mean |
|---|---|---|---|
| | mol % +3 | Mean | mol % −3 |
| Radicals | STD | mol % | STD |
| t-mannopyranose | 4.14% | 3.57% | 3.00% |
| t-glucopyranose | 17.59% | 15.58% | 13.58% |
| t-galactofuranose | 4.20% | 3.59% | 2.98% |
| t-glucofuranose | 0.73% | 0.15% | 0.00% |
| t-galactopyranose | 11.69% | 10.67% | 9.65% |
| 3-glucopyranose | 4.61% | 4.22% | 3.84% |
| 2-mannopyranose and/or | 1.99% | 1.41% | 0.83% |
| 3-mannopyranose | | | |
| 2-glucopyranose | 3.03% | 2.88% | 2.72% |
| 2-galactofuranose and/or | 1.78% | 1.30% | 0.83% |
| 2-glucofuranose and/or | | | |
| 3-glucofuranose | | | |
| 3-galactopyranose | 3.77% | 3.28% | 2.79% |
| 3-galactofuranose | 2.24% | 1.92% | 1.60% |
| 6-mannopyranose | 2.47% | 2.28% | 2.08% |
| 2-galactopyranose | 2.42% | 2.03% | 1.65% |
| 6-glucopyranose | 11.06% | 10.29% | 9.53% |
| 4-galactopyranose and/or | 3.06% | 2.75% | 2.45% |
| 5-galactofuranose | | | |
| 4-glucopyranose and/or | 3.89% | 3.45% | 3.00% |
| 5-glucofuranose and/or | | | |
| 6-mannofuranose | | | |
| 2,3-galactofuranose | 0.42% | 0.05% | 0.00% |
| 6-glucofuranose | 0.77% | 0.31% | 0.00% |
| 6-galactofuranose | 2.68% | 2.50% | 2.31% |
| 6-galactopyranose | 8.75% | 7.90% | 7.06% |
| 3,4-galactopyranose and/or | 1.08% | 0.97% | 0.86% |
| 3,5-galactofuranose and/or | | | |
| 2,3-galactopyranose | | | |
| 3,4-glucopyranose and/or | 0.75% | 0.61% | 0.47% |
| 3,5-glucofuranose | | | |
| 2,3-glucopyranose | 2.11% | 0.89% | 0.00% |
| 2,4-mannopyranose and/or | 0.85% | 0.21% | 0.00% |
| 2,5-mannofuranose | | | |
| 2,4-glucopyranose and/or | 1.88% | 1.17% | 0.45% |
| 2,5-glucofuranose and/or | | | |
| 2,4-galactopyranose and/or | | | |
| 2,5-galactofuranose | | | |
| 4,6-mannopyranose and/or | 0.70% | 0.53% | 0.35% |
| 5,6-mannofuranose | | | |
| 3,6-glucopyranose | 2.93% | 2.47% | 2.02% |
| 3,6-mannopyranose | 0.66% | 0.54% | 0.43% |
| 2,6-mannopyranose | 0.51% | 0.45% | 0.39% |
| 3,6-glucofuranose | 0.33% | 0.12% | 0.00% |
| 2,6-glucopyranose and/or | | | |
| 4,6-glucopyranose and/or | 2.55% | 2.15% | |
| 5,6-glucofuranose | | | 1.74% |
| 3,6-galactofuranose | 1.16% | 1.01% | 0.86% |
| 4,6-galactopyranose and/or | 2.86% | 2.47% | 2.09% |
| 5,6-galactofuranose | | | |
| 3,6-galactopyranose | 2.73% | 2.36% | 1.99% |
| 2,6-galactopyranose | 1.49% | 1.24% | 0.99% |
| 3,4,6-mannopyranose and/or | | | |
| 3,5,6-mannofuranose and/or | 0.12% | 0.01% | 0.00% |
| 2,3,6-mannofuranose | | | |
| 3,4,6-galactopyranose and/or | 0.96% | 0.75% | 0.54% |
| 3,5,6-galactofuranose and/or | | | |
| 2,3,6-galactofuranose | | | |
| 3,4,6-glucopyranose and/or | 0.63% | 0.28% | 0.00% |
| 3,5,6-glucofuranose | | | |
| 2,3,6-mannopyranose | 0.34% | 0.12% | 0.00% |
| 2,4,6-glucopyranose and/or | 0.80% | 0.40% | 0.00% |
| 2,5,6-glucofuranose | | | |
| 2,3,6-galactopyranose and/or | 1.31% | 0.59% | 0.00% |
| 2,4,6-galactopyranose and/or | | | |
| 2,5,6-galactofuranose | | | |
| 2,4,6-galactopyranose and/or | 0.92% | 0.15% | 0.00% |
| 2,5,6-galactofuranose | | | |
| 2,3,6-glucopyranose | 0.74% | 0.37% | 0.00% |

### Example 9. HSQC NMR Analysis Procedure Using a Varian Unity Inova NMR Machine

A determination of HSQC NMR spectra of samples of the selected oligosaccharide composition, as produced by a process as described in Example 1 (Marathon C catalyst), was performed using a Varian Unity Inova NMR, according to the protocol described below.

### Method

### Sample Preparation:

25 mg of a previously lyophilized solid sample was dissolved in 300 μL of D2O with 0.1% acetone as internal standard. The solution was then placed into a 3 mm NMR tube.

### Nmr Experiment:

The sample was analyzed in a Varian Unity Inova operating at 499.83 MHz (125.69 MHz 13C) equipped with a XDB broadband probe with Z-axis gradient, tuned to 13C, and operating at 25° C. The sample was subjected to a heteroatomic single quantum coherence (HSQC), echo-antiecho, with gradient selection HSQCETGP pulse sequence experiment using the following acquisition and processing parameters in Table 3:

TABLE 3
Acquisition Parameter

| | |
|---|---|
| Number of Scans | 8 |
| Recycle Delay | 1 second |
| Number of data points | 596 × 600 |
| Sweep Width (ppm) | 4 ppm × 110 ppm |
| Carrier Frequency | 4.0 ppm-65 ppm |
| CH Coupling Constant | 146 Hz |

Processing Parameter

| | |
|---|---|
| Size of Matrix | 1024 × 2048 |
| Window Function | Gaussian (7.66, 26.48 Hz) |
| Baseline correction | No correction |

### Spectral Analysis:

The resulting spectrum was analyzed using the MNova software package from Mestrelab Research (Santiago de Compostela, Spain). The spectrum was referenced to the internal acetone signal (1H-2.22 ppm; 13C-30.8 ppm) and phased using the Regions2D method in both the F2 and F1 dimension. Apodization using 90 degree shifted sine was applied in both the F2 and F1 dimension. Individual signals (C—H correlations) were quantified by integration of their respective peaks using “predefined integral regions” with elliptical integration shapes. The resulting table of integral regions and values were normalized to a sum of 100 in order for the value to represent a percentage of the total. Peak integral regions were selected to avoid peaks associated with monomers.

### Results

Ten batches of the selected oligosaccharide composition analyzed by SEC as described in Example 10, and produced according to the process of Example 1 (Marathon C catalyst), were analyzed using the above NMR methods. Collectively, these batches comprised the following NMR peak signals (Table 4)

TABLE 4
HSQC NMR peaks of the selected oligosaccharide composition

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | from | to |
| 1 | 3.68 | 3.61 | 3.75 | 63.42 | 62.64 | 64.20 |
| 2 | 3.75 | 3.72 | 3.78 | 66.06 | 65.50 | 66.62 |
| 3 | 3.97 | 3.94 | 4.00 | 66.15 | 65.81 | 66.49 |
| 4 | 3.96 | 3.94 | 3.98 | 69.28 | 69.04 | 69.52 |
| 5 | 3.96 | 3.9 | 4.03 | 70.62 | 70.20 | 71.05 |
| 6 | 3.92 | 3.9 | 3.94 | 71.26 | 71.02 | 71.50 |
| 7 | 3.55 | 3.51 | 3.59 | 71.34 | 71.06 | 71.62 |
| 8 | 3.97 | 3.94 | 4.00 | 71.56 | 71.29 | 71.84 |
| 9 | 3.72 | 3.67 | 3.77 | 72.35 | 71.95 | 72.74 |
| 15 | 4.44 | 4.41 | 4.46 | 103.86 | 103.56 | 104.15 |
| 10 | 3.33 | 3.27 | 3.4 | 73.74 | 73.26 | 74.22 |
| 14 | 4.5 | 4.47 | 4.54 | 103.29 | 102.87 | 103.70 |
| 11 | 4.06 | 4.04 | 4.09 | 77.34 | 76.89 | 77.78 |
| 12 | 4.11 | 4.08 | 4.14 | 81.59 | 81.16 | 82.01 |
| 13 | 4.96 | 4.92 | 5.01 | 98.7 | 98.02 | 99.39 |

The relative size of each of the peaks (AUC) collected for the NMR spectra of the selected oligosaccharide composition was further determined, as shown below:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 20.38-25.74 |
| 2 | 3.75 | 66.06 | 3.69-6.38 |
| 3 | 3.97 | 66.15 | 2.21-3.40 |
| 4 | 3.96 | 69.28 | 1.46-3.71 |
| 5 | 3.96 | 70.62 | 9.28-10.71 |
| 6 | 3.92 | 71.26 | 1.52-2.03 |
| 7 | 3.55 | 71.34 | 3.40-6.13 |
| 8 | 3.97 | 71.56 | 3.40-4.41 |
| 9 | 3.72 | 72.35 | 5.66-10.14 |
| 10 | 3.33 | 73.74 | 10.21-12.09 |
| 11 | 4.06 | 77.34 | 3.68-4.50 |
| 12 | 4.11 | 81.59 | 3.10-3.82 |
| 13 | 4.96 | 98.7 | 10.65-12.31 |
| 14 | 4.5 | 103.29 | 5.03-6.41 |
| 15 | 4.44 | 103.86 | 1.84-2.44 |

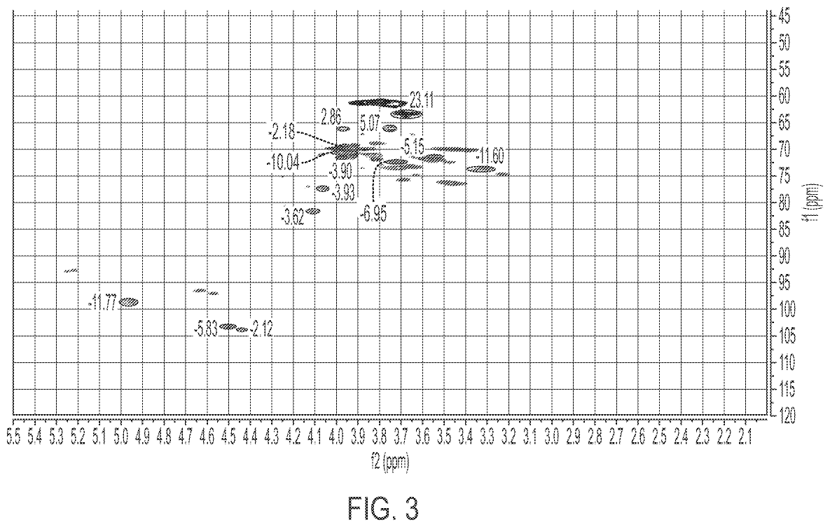

A representative HSQC NMR spectra of the selected oligosaccharide composition is provided in FIG. 3.

Twenty-three batches of the selected oligosaccharide composition produced according to the process of Example 2 (citric acid catalyst) and analyzed by SEC as described in Example 12, were analyzed using the above NMR methods. Collectively, these batches comprised the NMR peak signals as shown in Table 4. The relative size of each of the peaks (AUC) collected for the NMR spectra of the selected oligosaccharide composition produced according to the process as described in Example 2 was further determined, as shown below:

| | Center Position | | |
|---|---|---|---|
| | (ppm) | | Area under the curve (AUC) |
| Signal | ¹H | ¹³C | (% of total areas of all signals) |
| 1 | 3.68 | 63.42 | 21.57-25.73 |
| 2 | 3.75 | 66.06 | 3.87-5.54 |
| 3 | 3.97 | 66.15 | 2.63-3.43 |
| 4 | 3.96 | 69.28 | 1.28-3.86 |
| 5 | 3.96 | 70.62 | 9.08-11.04 |
| 6 | 3.92 | 71.26 | 1.49-2.70 |
| 7 | 3.55 | 71.34 | 4.48-5.90 |
| 8 | 3.97 | 71.56 | 3.07-3.99 |
| 9 | 3.72 | 72.35 | 6.87-8.66 |
| 10 | 3.33 | 73.74 | 10.79-11.70 |
| 11 | 4.06 | 77.34 | 3.28-3.99 |
| 12 | 4.11 | 81.59 | 2.82-3.39 |
| 13 | 4.96 | 98.7 | 10.60-12.69 |
| 14 | 4.5 | 103.29 | 4.90-6.25 |
| 15 | 4.44 | 103.86 | 1.81-2.42 |

Peaks of the selected oligosaccharide composition were assigned using commercially sourced dimers from Carbosynth Inc. Assignments were based on strategies like the “1H NMR structural-reporter-group concept” (Leeuwen et al. Carbohydrate Research 343 (2008), 1114-1119). Dimers used in the analysis included: Glup-b (1-2)-Glup, Glup-a (1-2)-Glup, Glup-b (1-3)-Glup, Glup-b (1-4)-Glup, Glup-a (1-4)-Glup, Glup-b (1-6)-Glup, Glup-a (1-6)-Glup, Glup-a,b (1-1)-Glup, Glup-a,a (1-1)-Glup, Galp-a (1-6)-Glup, Galp-b (1-4)-Glup, Galp-b (1-6)-Galp, Galp-b (1-4)-Galp, Galp-b (1-3)-Galp, Galp-a (1-3)-Galp, Galp-b (1-2)-Galp, and Galp-a (1-2)-Galp. Linkage abundances were compared with permethylation data and cross referenced with literature values and compositionally pure selected oligosaccharide compositions. Some peak assignments were confirmed by 2D NMR analysis at the Complex Carbohydrate Research Center (CCRC) at the University of Georgia.

### Example 10. Size Exclusion Chromatography Method

The weight-average molecular weight (MWw), number-average molecular weight (MWn), and polydispersity index (PDI) of batches and samples of the selected oligosaccharide composition, as produced by the process in Example 1 (Marathon C catalyst), were determined by SEC HPLC

### Method

These methods involved the use of an Agilent 1100 with refractive index (RI) detector equipped with a guard column (Shodex SUGAR SP-G 6B Guard Column 6×50 mm, 10 μm) and two chromatography columns in series: 1) Shodex OHpak SB-802 HQ, 8.0×300 mm, 8 μm, P/N F6429100; 2) Shodex OHpak SB-803 HQ. 8.0×300 mm, 6 μm, P/N F6429102.

The mobile phase (0.1 M NaNO₃) was prepared by weighing 17 g of NaNO₃ (ACS grade reagent) and dissolving in 2000 mL of deionized (DI) water (from MilliQ water filter). The solution was filtered through a 0.2 μm filter.

Polymer standard solutions (10.0 mg/mL) of each of D-(+) Glucose Mp 180, Carbosynth Ltd Standard, or equivalent (CAS #50-99-7); Maltose Mp 342, Carbosynth Ltd Standard, or equivalent (CAS #69-79-4); Maltotetraose Mp 667, Carbosynth Ltd Standard, or equivalent (CAS #34612-38-9); Maltooctaose Mp 1315, Carbosynth Ltd Standard, or equivalent (CAS #6156-84-9); Nominal Mp 6100 Pullulan Standard, PSS #PPS-pul6k; Nominal Mp 9600 Pullulan Standard, PSS #PPS-pul10k; and Nominal Mp 22000 Pullulan Standard, PSS #PPS-pul22k were prepared by weighing 20 mg of a standard into a separate 20 mL scintillation vial and adding 2.0 mL of DI water to each vial. A polymer solution mixture #1 was prepared by weighing 10 mg of each standard of glucose, maltose, maltooctaose and Mp 9600 into an HPLC vial, adding 1.0 mL of diluent and mixing well. A polymer solution mixture #1 was prepared by weighing 10 mg of each standard of maltotetraose, Mp 6100 and Mp 21100 into an HPLC vial, adding 1.0 mL of diluent and mixing well.

Sample A was prepared in duplicate. Approximately 300 mg of oligosaccharide sample was weighed into a 20 mL scintillation vial and 10 mL of DI water was added. The solution was mixed and filtered through a PES syringe filter with a 0.2 μm polyethersulfone membrane.

Sample B was prepared in duplicate. Approximately 220 mg of oligosaccharide sample was weighed into a 20 mL scintillation vial and 10 mL of DI-water was added. The solution was mixed and filtered a PES syringe filter with a 0.2 μm polyethersulfone membrane.

The flow rate was set to 0.7 mL/min at least 2 hours before running samples with the column temperature set to 65° C. and the RI detector temperature set to 50° C. with the RI detector purge turned on.

Before running samples wherein the injection volume for all samples was 10 μL and run time was 40 minutes, the detector purge was turned off and the pump was run at 0.7 mL/min until an acceptable baseline was obtained.

A blank sample consisting of DI water was run. Samples of standard mixtures #1 and #2 were run. Sample A was run. Sample B was run.

The peaks between 18 and 25.5 minutes were integrated. The monomer and the broad peak (the product) were integrated. The calibration curve fit type in Empower 3 software was set to 3^{rd} order. The molecular weight distributions and polydispersity were calculated using Empower 3 software for the broad peak. The Mw, Mn and polydispersity of the product peak (DP2+) were determined using these methods.

### Results

Fourteen batches of the selected oligosaccharide composition produced using the process in Example 1 (Marathon C catalyst) were analyzed using the above SEC methods. The batches of oligosaccharide composition comprised oligosaccharides with an average MWw of 2074 g/mol (ranging from 1905-2286 g/mol), an average MWn of 1097 g/mol (ranging from 1033-1184 g/mol), and an average PDI of 1.9 (ranging from 1.84-1.97). Assayed batches comprised a DP2+ of 91.1% (DP2+ ranging from 86.3-95.9%) and about 8.9% monomer on average (ranging from 4.1-13.8% monomer). Assayed batches had an average degree of polymerization (DP) of 12.7 (ranging from 11.6-14.0).

Twenty-three batches of the selected oligosaccharide composition produced using the process in Example 2 (citric acid catalyst) were analyzed using the above SEC methods. The batches of oligosaccharide composition comprised oligosaccharides with an average MWw of 1998 g/mol (ranging from 1863-2268 g/mol), an average MWn of 1030 g/mol (ranging from 984-1106.00 g/mol), and an average PDI of 1.94 (ranging from 1.88-2.05). Assayed batches comprised a DP2+ of 87.3% (DP2+ ranging from 83.6-91.0%) and about 12.7% monomer on average (ranging from 9.0-16.4% monomer). Assayed batches had an average degree of polymerization (DP) of 12.2 (ranging from 11.4-13.9).

### Example 11. SEC HPLC Methodology for Determination of Impurities

The presence of residual organic acid impurities and related substances of batches and samples of the selected oligosaccharide composition, as produced by the processes in Example 1 (Marathon C catalyst), were determined by SEC HPLC.

### Methods

These methods involved the use of an Agilent 1100 with refractive index (RI) detector equipped with a guard column (Bio-Rad MicroGuard Cation H+ Cartridge, PIN 125-0129, or equivalent) and a Bio-Rad Aminex HPX-87H, 300×7.8 mm, 9 μm, PIN 125-0140 column, or equivalent.

The mobile phase (25 mM H₂SO₄ in water) was prepared by filling a bottle with 2000 mL DI-water and slowly adding 2.7 mL of H₂SO₄. The solution was filtered through a 0.2 μm filter.

A standard solution was prepared by measuring 50±2 mg of reference standard into a 100-mL volumetric flask, adding mobile phase to 100-mL mark and mixing well.

A sample of the selected oligosaccharide composition (Sample A) was prepared in duplicate. Approximately 1000 mg of oligosaccharide sample was weighed into a 10 mL volumetric flask and mobile phase was added up to the mark. The solution was mixed and filtered through a PES syringe filter with a 0.2 μm polyethersulfone membrane.

A sample of the selected oligosaccharide composition (Sample B) was prepared in duplicate. Approximately 700 mg of oligosaccharide sample was weighed into a 10 mL volumetric flask and mobile phase was added up to the mark. The solution was mixed and filtered through a PES syringe filter with a 0.2 μm polyethersulfone membrane.

The flow rate was set to 0.65 mL/min at least 2 hours before running samples with the column temperature set to 50° C. and the RI detector temperature set to 50° C. with the RI detector purge turned on.

Before running samples wherein the injection volume for all samples was 50 μL and run time was 40 minutes, the detector purge was turned off and the pump was run at 0.65 mL/min until an acceptable baseline was obtained.

A blank sample consisting of DI water was run. The standard, sample A, and sample B were each independently run.

The peaks at 7.5 min (Glucuronic acid), 9.4 min (Maleic Acid), 11.3 min (Levoglucosan), 11.9 min (Lactic Acid), 13.1 min (Formic Acid), 14.2 min (Acetic Acid), 15.5 min (Levulinic Acid), 31.8 min (HMF), and 8.3 min (Glucose) were integrated. The calibration curve fit type in Empower 3 software was set to 3rd order.

### Results

Ten batches of the selected oligosaccharide, as produced by the process in Example 1 (Marathon C catalyst), were tested using the method above. The selected oligosaccharide composition comprised 0.35% w/w (+0.05%) levoglucosan, 0.03% w/w (+0.01%) lactic acid, and 0.06% w/w (+0.01%) formic acid. Samples of the selected oligosaccharide composition comprised 0.28-0.43% w/w levoglucosan, 0.00-0.03% w/w lactic acid, and 0.05-0.07% w/w formic acid.

Batches of the selected oligosaccharide, as produced by the process in Example 2 (citric acid catalyst), were tested using the method above. The selected oligosaccharide composition comprised 0.47% w/w (+0.02%) levoglucosan (23 batches of the selected oligosaccharide), 0.01% w/w lactic acid (11 batches of the selected oligosaccharide), 0.02% w/w formic acid (12 batches of the selected oligosaccharide), and 0.02% w/w citric acid (23 batches of the selected oligosaccharide). Samples of the selected oligosaccharide composition comprised 0.43-0.51% w/w levoglucosan, 0.01-0.02% w/w lactic acid, 0.00-0.03% w/w formic acid, and 0.00-0.03% w/w citric acid.

### Example 12. SEC HPLC Methodology for Determination of DP1-DP7

The relative amounts of oligosaccharides with a degree of polymerization (DP) of 1, 2, and 3+ in batches and samples of the selected oligosaccharide composition, as produced by the processes in Example 1 were determined by SEC HPLC.

### Methods

These methods involved the use of an Agilent 1100 with refractive index (RI) detector equipped with a guard column (Shodex SUGAR SP-G 6B Guard Column 6×50 mm, 10 μm, P/N F6700081, or equivalent) and a chromatography column (Shodex Sugar SP0810, 8.0×300 mm, 8 μm, P/N F6378105, or equivalent).

The mobile phase (0.1 M NaNO₃) was prepared by weighing 42.5 g of NaNO₃ (ACS grade reagent) and dissolving in 5000 mL of deionized (DI) water (from MiliQ water filter). The solution was filtered through a 0.2 μm filter.

Polymer standard solutions (10.0 mg/mL) of each of D-(+) Glucose Mp 180, Carbosynth Ltd Standard, or equivalent (CAS #50-99-7) (DP1); Maltose Mp 342, Carbosynth Ltd Standard, or equivalent (CAS #69-79-4) (DP2); Maltotriose Mp 504, Carbosynth Ltd Standard, or equivalent (CAS #1109-28-0) (DP3); Maltotetraose Mp 667, Carbosynth Ltd Standard, or equivalent (CAS #34612-38-9) (DP4); Maltopentaose Mp 828, Carbosynth Ltd Standard, or equivalent (CAS #34620-76-3) (DP5); Maltohexaose Mp 990, Carbosynth Ltd Standard, or equivalent (CAS #34620-77-4) (DP6); Maltoheptaose Mp 1153, Carbosynth Ltd Standard, or equivalent (CAS #34620-78-5) (DP7); and Maltooctaose Mp 1315, Carbosynth Ltd Standard, or equivalent (CAS #6156-84-9) (DP8), were prepared by weighing 10 mg of a standard into an individual 1.5 mL centrifuge tube and adding DI water to make 10 mg/mL solution.

Samples of the selected oligosaccharide composition were prepared as 10 mg/mL concentrated samples or dilute aqueous samples to 2.5-3.5 Brix.

The flow rate was set to 1.0 mL/min at least 2 hours before running samples with the column temperature set to 70° C. and the RI detector temperature set to 40° C. with the RI detector purge turned on.

Before running samples wherein the injection volume for all samples was 5 μL and run time was 15 minutes, the detector purge was turned off and the pump was run at 1.0 mL/min until an acceptable baseline was obtained.

A blank sample consisting of DI water, individual standards, and sample were independently run.

Each peak between 4 and 9.2 minutes in the sample run, corresponding to individual standards, was integrated. The calibration curve fit type in Empower 3 software was set to 3rd order. The DP1, DP2, and DP3+ values of the samples (samples of selected oligosaccharide composition) were determined using these methods.

### Results

Ten samples of selected oligosaccharide composition produced using the process in Example 1 were assayed using this method. The selected oligosaccharide composition comprised 5.24% (+0.35%) monomer (DP1), 7.52% (+0.44%) disaccharide (DP2), and 87.25% (+0.78%) oligomers having at least three linked monomer units (DP3+).

### Example 13. Determination of Total Dietary Fiber

The amount of dietary fiber in batches of the selected oligosaccharide composition, as produced by the process in Example 1 (Marathon C catalyst) were measured according to the methods of AOAC 2011.25 (AOAC International, AOAC Official Method 2011.25). The average amount of total dietary fiber was 87.44% (on dry basis) across 10 batches (ranging from 84.9-90.5%). The percent Dextrose Equivalent (DE) (dry basis) of these oligosaccharide batches was also measured according to the Food Chemicals Codex (FCC). The average amount of dextrose equivalent (on dry basis) was 16.60% across two batches (one at 15.10% DE and the other at 18.10% DE).

The amount of dietary fiber and dextrose equivalents (DE) in batches of the selected oligosaccharide composition, as produced by the process in Example 2 (citric acid catalyst) were measured according to the methods of AOAC 2011.25 (AOAC International, AOAC Official Method 2011.25). The percent Dextrose Equivalent (DE) (dry basis) of these oligosaccharide batches was also measured according to the Food Chemicals Codex (FCC). The average amount of total dietary fiber was 64.14% (on dry basis) across fourteen batches (ranging from 47.10-73.10%). The average amount of dextrose equivalent (on dry basis) was 20.60% across two batches (one at 18.60% DE and the other at 22.60% DE).

### Example 14. Assessment of Selected Oligosaccharide Compositions in Fecal Suspensions from Healthy Subjects

The ability of the selected oligosaccharide composition comprised of a plurality of oligosaccharides selected from Formula (I), Formula (II), and Formula (III) as produced by a similar process as described in Examples 1-3 to reduce pathogen growth and promote commensal growth in microbiome samples from fecal suspensions of healthy subjects was assessed.

Fecal samples from healthy subjects were prepared as described in Example 8.

A single strain of vancomycin-resistant Enterococcacede (VRE) was grown in isolation overnight in MM with 0.5% D-glucose in a COY chamber. On the day of the experiment, aliquots of the overnight cultures were washed with PBS and the optical density (OD₆₀₀) of the cultures was measured. The culture was adjusted to OD₆₀₀ of 0.1 in MM and added to the 1% fecal suspensions. Fecal suspensions mixed with vancomycin-resistant Enterococcaceae (VRE) were then subjected to 16S sequencing to determine the initial relative abundance of pathogen and commensal bacteria. The cultures were then added to 96-well microplates with one of the following carbon sources (final concentration of 0.5% w/v) in each well: maltodextrin, fructooligosaccharide, a sample of the selected oligosaccharide composition, or water (negative control, i.e., no carbon source). These microplates were then incubated at 37° C. in the COY chamber for a total of 45 hours, with each experimental condition being tested in three replicates on each plate.

At the end of the 45-hour incubation, a sample of the culture from each well was subjected to 16S sequencing to determine the final relative abundance of pathogen and commensal bacteria in the community after intervention with oligosaccharide composition, as described in Example 8.

For the 16S sequencing, genomic DNA was extracted from the fecal suspensions and variable region 4 of the 16S rRNA gene was amplified and sequenced (Earth Microbiome Project protocol www.carthmicrobiome.org/emp-standard-protocols/16s/and Caporaso J G et al. Ultra-high-throughput microbial community analysis on the Illumina HiSeq and MiSeq platforms. ISME J. (2012) August; 6 (8): 1621-4). Raw sequences were demultiplexed, and each sample was processed separately with UNOISE2 (Robert Edgar UNOISE2: improved error-correction for Illumina 16S and ITS amplicon sequencing. bioRxiv (2016) October 15). Reads from 16S rRNA amplicon sequencing data were rarefied to 5000 reads, without replacement, and resulting OTU table used in downstream calculations.

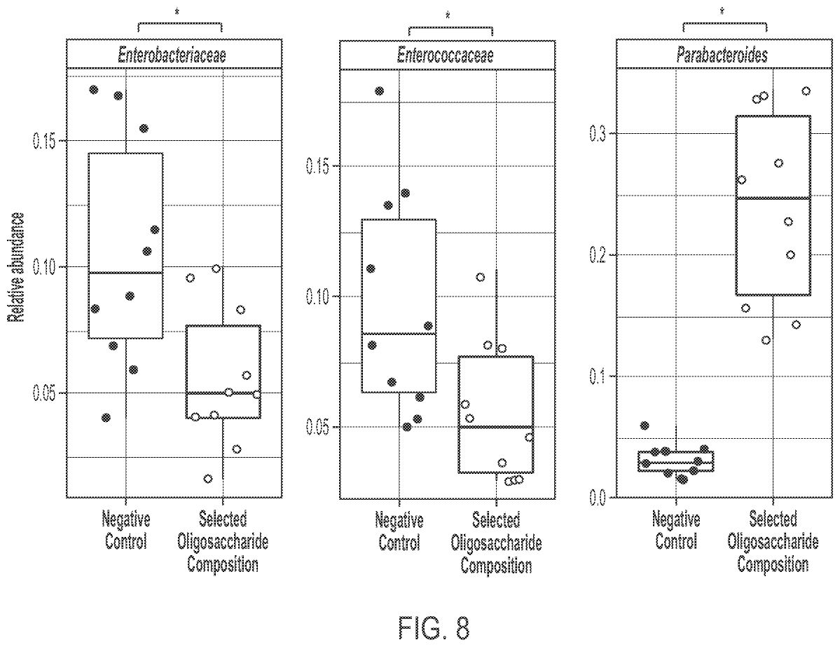

The selected oligosaccharide composition significantly reduced the abundance of pathogens (Enterobacteriaceae and Enterococcaceae) (FIG. 8) in spiked fecal suspensions from healthy subjects, as assessed by 16S sequencing and relative to negative control (fecal suspensions spiked with water).

The selected oligosaccharide composition significantly increased the abundance of commensal bacteria (Parabacteroides) (FIG. 8) in spiked fecal suspensions from healthy subjects, as assessed by 16S sequencing and relative to negative control (fecal suspensions spiked with water).

This demonstrates that the selected oligosaccharide composition comprised of a plurality of oligosaccharides selected from Formula (I), Formula (II), and Formula (III) as produced by a similar process as described in Examples 1-3 is capable of reducing or preventing the growth of pathogens and promoting or increasing the growth of commensal bacteria in a medically relevant model.

### Example 15. Assessment of Selected Oligosaccharide Compositions in Commensal Strains

The ability of the selected oligosaccharide composition comprised of a plurality of oligosaccharides selected from Formula (I), Formula (II), and Formula (III) as produced by a similar process as described in Examples 1-3 to promote growth of single commensal strains was assessed.

Single strains of Parabacteroides species (2 total) and Bacteroides species (7 total) were cultured for 16 hours at 37° C. in a COY anaerobic chamber. 1 mL of each culture was washed with phosphate buffered saline and the optical density (OD₆₀₀) of each culture was measured. Each culture was adjusted to OD₆₀₀ of 0.02 in filter-sterilized medium containing 10 g/L tryptone peptone, 5 g/L yeast extract, 4.1 mM L-cysteine, 100 mM potassium phosphate buffer (pH 7.2), 0.008 mM magnesium sulfate, 4.8 mM sodium bicarbonate, 1.37 mM sodium chloride, 5.8 mM vitamin K, 0.8% calcium chloride, 1.44 mM iron (II) sulfate heptahydrate, 4 mM resazurin, 0.1% histidine-hematin, 1% ATCC trace mineral supplement, 1% ATCC vitamin supplement, 29.7 mM acetic acid, 0.9 mM isovaleric acid, 8.1 mM propionic acid, and 4.4 mM N-butyric acid, pH 7.0.

Inside of the COY anaerobic chamber, the normalized single strain cultures were added to 96 well flat bottom microplates with either D-glucose (positive control) or the selected oligosaccharide composition as an added carbon source to each well. A negative control in which water was added to the single strain cultures without an added carbon source was also performed. The final concentration of the carbon source (glucose or the selected oligosaccharide composition) in the assay was 0.5%. Each carbon source (glucose or the selected oligosaccharide composition) was represented 3 times within each assay plate (i.e., with each single strain). Plates are covered with a breathable membrane and were incubated at 37° C. in the COY anaerobic chamber for a total of 45 hours. OD₆₀₀ was measured every 15 minutes. These OD₆₀₀ reads generated growth curves for each assay plate using the Biotek BioStack microplate stacker and Bioteck Powerwave HT microplate spectrophotometer. The median OD₆₀₀ normalized to the negative control was determined for the growth curves generated for each strain and compound combination using the growthcurver library in Rstudio.

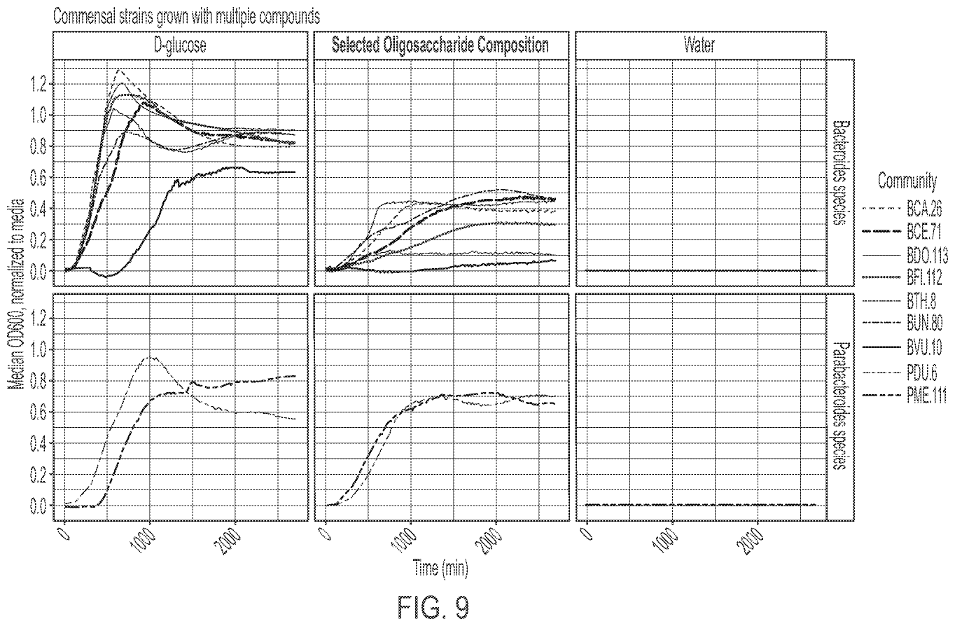

The selected oligosaccharide composition supported the growth of Parabacteroides species and Bacteroides species (FIG. 9). For all species tested, the maximum ability to grow under our conditions was defined by the positive control (D-glucose) and the minimum ability of a strain to grow on the background media was defined by the negative control (water). The growth of Parabacteroides distasonis (PDI.6) and Parabacteroides merdae (PME.111) were supported by the selected oligosaccharide composition at similar levels of growth compared to D-glucose, indicating almost full utilization of the selected oligosaccharide composition as a carbon source. The growth of Bacteroides thetaiotaomicron (BTH.8), Bacteroides uniformis (BUN.80), Bacteroides caccae (BCA.26), Bacteroides cellulosilyticus (BCE.71) were supported by the selected oligosaccharide composition at high levels. The growth of Bacteroides finegoldii (BFI.112) and Bacteroides dorea (BDO.113) were also supported by the selected oligosaccharide composition relative to negative controls. Bacteroides vulgatus (BVU.10) was at least minimally supported by the selected oligosaccharide composition in this experiment.

### Example 16. Effects of Selected Oligosaccharide in RSV and Influenza Animal Models

To assess whether the selected oligosaccharide composition is beneficial in respiratory virus infections, e.g., RSV and influenza which are associated with COPD exacerbations, including AECOPD, pilot studies in mouse models of influenza and RSV (respiratory syncytial virus) infection were performed. The microbiome has previously been implicated in protective immune responses to influenza and RSV infection in mice (Antunes K H, et al. Microbiota-derived acetate protects against respiratory syncytial virus infection through a GPR43-type 1 interferon response. Nat Commun. 2019; 10 (1): 3273; Trompette A, et al. Dietary Fiber Confers Protection against Flu by Shaping Ly6c-Patrolling Monocyte Hematopoiesis and CD8+ T Cell Metabolism. Immunity. 2018; 48 (5): 992-1005.e8; Bradley K C, et al. Microbiota-Driven Tonic Interferon Signals in Lung Stromal Cells Protect from Influenza Virus Infection. Cell Rep. 2019; 28 (1): 245-256.e4), suggesting that effects of microbiome modulation by the selected oligosaccharide can be evaluated in these models. The pilot studies were primarily performed to test experimental parameters such as dosing, length of treatment and route of administration and thus any data were obtained under conditions that were not optimized. Additionally, differences between the gastrointestinal microbiota of humans and mice pose experimental challenges (Nagpal R, et al. Comparative Microbiome Signatures and Short-Chain Fatty Acids in Mouse, Rat, Non-human Primate, and Human Feces. Front Microbiol. 2018; 9:2897; Hugenholtz F, de Vos W M. Mouse models for human intestinal microbiota research: a critical evaluation. Cell Mol Life Sci. 2018; 75 (1): 149-160). For example, species of bacteria in the Parabacteroides genus that are believed to utilize the selected oligosaccharide as a carbon source in humans are usually present only in low abundance in the mouse microbiome (Nagpal R, et al. Front Microbiol. 2018; 9:2897; Xiao L, et al. A catalog of the mouse gut metagenome. Nat Biotechnol. 2015; 33 (10): 1103-1108). Despite these challenges and lack of optimization of experimental parameters, preliminary, limited data obtained in mice suggest beneficial effects of the selected oligosaccharide on infections caused by respiratory viruses such as, e.g., influenza and RSV infection.

Mouse models were adapted from existing publications (Antunes K H, et al. Nat Commun. 2019; 10 (1): 3273; Trompette A, et al. Immunity. 2018; 48 (5): 992-1005.e8). To better control dosing, the selected oligosaccharide was administered by oral gavage instead of in animal chow.

The design for the RSV pilot study is summarized in Table 5. Briefly, female BALB/c mice between 6-8 weeks of age were weighed one day prior to study start and randomized into 3 groups of n=10, such that each group had approximately the same average weight. Mouse body weight was recorded every 2-3 days before viral challenge and every day after. Clinical symptoms were monitored daily. Fecal pellets were collected on days-8, 0, 2 and 5. Groups 1 and 2 received 200 μL of vehicle (water) once a day (QD) from D-7 to D4 via oral gavage (PO). Group 3 received the selected oligosaccharide at 3000 mg/kg/day QD from D-7 to D4 PO, administered as the appropriate volume of a 0.3 g/mL solution. On Day 0, RSV-A2 was administered to Groups 2 and 3 by intranasal inoculation with 50 μL of a viral stock solution at 8.5×10⁵ PFU/mL. Group 1 was not infected. The study was terminated at D5. All animals were euthanized, and lungs were harvested for viral plaque assay, histology, and cytokine profiling.

TABLE 5
Summary of the RSV pilot study

| | | | Challenge | | Dosing | |
|---|---|---|---|---|---|---|
| | Group | Group | virus | | Route and | Endpoints |
| Group | size | Description | (Intranasal) | Treatment | Frequency | and Analyses |
| 1 | 10 | Mock | — | Vehicle (water) | PO, QD | Body weight |
| 2 | 10 | RSV negative | RSV-A2 | Vehicle (water) | PO, QD | Viral titers |
| | | control | | | | in the lung |
| 3 | 10 | RSV + selected | RSV-A2 | selected | PO, QD | Lung |
| | | oligosaccharide | | oligosaccharide | | histopathology |
| | | (3000 mpk/day) | | | | Cytokine |
| | | | | | | profiling in |
| | | | | | | lung homogenates |

The design for the influenza pilot study is summarized in Table 6. Briefly, female BALB/c mice between 6-8 weeks of age were weighed one day prior to study start and randomized into 3 groups of n=16, such that each group had approximately the same average weight. All mice were treated with saline or the selected oligosaccharide twice daily (BID) starting 7 days prior to virus inoculation and continuing through end of study. The experimental animals were inoculated intranasally on day 0 with 300 PFU/mouse in a volume of 50 μL. Fecal pellets were collected on days −7, 0, 6, 14 for the health monitoring cohorts and days −7, 0, 6 for the sample collection cohorts. On day 6, animals from the sample collection cohorts were euthanized, and blood, bronchoalveolar (BAL) fluid, and lung samples were collected. On day 14, all the surviving mice from the health monitoring cohorts were euthanized.

TABLE 6
Summary of the influenza pilot study

| | | | | | Dosing | |
|---|---|---|---|---|---|---|
| | | Group | Challenge | | Route and | Endpoints |
| Animal Group | | size | virus | Treatment | Frequency | and Analyses |
| Mock | Health | 10 | Mock (50 μL | Saline | PO, BID, | Body weight |
| | monitoring | | PBS/mouse) | (0.9% NaCl) | day −7 to | Mortality |
| | | | | | day 14 | General health |
| | Sample | 6 | | | PO, BID, | Clinical score |
| | collection | | | | day −7 to | Lung viral titer |
| | | | | | day 6 | BAL fluid cytometry |
| Negative | Health | 10 | Influenza | Saline | PO, BID, | Lung pathology |
| Control | monitoring | | virus | (0.9% NaCl) | day −7 to | Cytokines in BAL |
| | | | A/PR/8/34 | | day 14 | fluid and serum |
| | Sample | 6 | (H1N1), | | PO, BID, | |
| | collection | | 300 PFU/mouse | | day −7 to | |
| | | | | | day 6 | |
| selected | Health | 10 | Influenza | selected | PO, 3000 | |
| oligosaccharide | monitoring | | virus | oligosaccharide | mg/kg/dose, | |
| | | | A/PR/8/34 | | BID, | |
| | | | (H1N1), | | day −7 to | |
| | | | 300 PFU/mouse | | day 14 | |
| | Sample | 6 | | | PO, 3000 | |
| | collection | | | | mg/kg/dose, | |
| | | | | | BID, | |
| | | | | | day −7 to | |
| | | | | | day 6 | |

In the RSV model, the selected oligosaccharide significantly decreased weight loss after RSV infection (p-value <0.05, linear mixed effect model) in comparison to the negative control group. The selected oligosaccharide also reduced lung viral load by approximately 0.16 log 10 PFU/g on average at day 5 (D5), though not statistically significant (p-value=0.076, student t test).

In the influenza model, the selected oligosaccharide did not show effects on body weight, clinical score, and survival compared to the negative control group, but in a flow cytometry analysis of bronchoalveolar (BAL) fluid, the selected oligosaccharide significantly reduced the percentage of infiltrating neutrophils, by about 37% (p-value: 0.035, student t test). Excessive neutrophil influx has been associated with dysregulated and damaging inflammation during lung infection (Antunes K H, et al. Nat Commun. 2019; 10 (1): 3273; Perrone L A, et al. H5N1 and 1918 Pandemic Influenza Virus Infection Results in Early and Excessive Infiltration of Macrophages and Neutrophils in the Lungs of Mice. Baric R S, ed. PLOS Pathog. 2008; 4 (8): e1000115).

Lung histopathology, cytokine analysis, and fecal microbiome analysis (to explore shifts in the mouse microbiome community) from both studies are analyzed. The preliminary, limited data obtained thus far for influenza and RSV together with the in-human data obtained for SARS-COV-2 suggest that the selected oligosaccharide may be able to improve clinical symptoms of, and reduce viral loads in respiratory viral infections in general, independent of the identity of the underlying respiratory virus. Influenza, RSV and other respiratory pathogens are also important triggers for severe exacerbation events in COPD thus the selected oligosaccharide may also be able to improve clinical symptoms in COPD patients. See, Papi, A. et al., “Infections and airway inflammation in chronic obstructive pulmonary disease severe exacerbations,” Am J Respir Crit Care Med. 2006 May 15; 173 (10): 1114-21.

### Example 17. Spray Drying Procedure

Individual batches of the selected oligosaccharide composition, as produced in Example 13 were spray-dried. The chamber of an SPX Anhydro MicraSpray 400 instrument equipped with a rotary atomizer was inerted to bring the oxygen concentration inside the chamber to <1%. The drying gas flow was started and set to 440 kg/hr. Once the drying gas flow was stable, the spray drying instrument was set to the following setpoints: (i) Spray dryer pressure setpoint=1050 mbar; (ii) Spray dryer chamber skin heater=60° C.; (iii) Recirculation fan pre-heater=20° C.; (iv) Steam heater inlet=90° C.; (v) Rotary atomizer speed=25000 RPM; (vi) Cyclone skin heater=90° C.; and (vii) Baghouse skin heater=40° C. Once the above spray dryer process parameters had stabilized, the blank solution (Purified Water) was turned on and set to 7.5 kg/hr., and the steam heater inlet temperature was increased to 145° C.

After the spray dryer outlet temperature had stabilized to around 90° C., the solvent feed was changed from blank solution (Purified Water) to feed solution (the selected oligosaccharide composition), and the feed rate was adjusted to 15.0 kg/hr. The spray-dried oligosaccharide composition was collected in an 800 L conical vessel as it was dried by the instrument.

### Example 18. HSQC NMR Analysis of a Sample of the Selected Oligosaccharide Composition Following Spray-Drying Using a Bruker NMR Machine

A batch of the selected oligosaccharide composition was further subjected to the spray drying procedure described in Example 17. Following the spray drying procedure, a sample of the spray dried composition was prepared for HSQC NMR analysis by dissolving 30 mg of the spray dried composition (after de-monomerization using an ethanol precipitation procedure) in 300 μL of D₂O with 0.1% acetone as internal standard. The solution was then placed into a 3 mm NMR tube and using the HSQC NMR methods described in Example 9. The relative size of each of the peaks collected for the NMR spectra of the analyzed batches of the selected oligosaccharide composition was determined, as shown below:

| | Center Position | | |
|---|---|---|---|
| | (ppm) | | Batch after spray-drying procedure |
| Signal | ¹H | ¹³C | (% of total areas of signals 1-15) |
| 1 | 3.68 | 63.42 | 25.34 |
| 2 | 3.75 | 66.06 | 4.54 |
| 3 | 3.97 | 66.15 | 3.32 |
| 4 | 3.96 | 69.28 | 3.10 |
| 5 | 3.96 | 70.62 | 10.08 |
| 6 | 3.92 | 71.26 | 2.15 |
| 7 | 3.55 | 71.34 | 5.27 |
| 8 | 3.97 | 71.56 | 3.49 |
| 9 | 3.72 | 72.35 | 7.33 |
| 10 | 3.33 | 73.74 | 10.94 |
| 11 | 4.06 | 77.34 | 3.86 |
| 12 | 4.11 | 81.59 | 3.20 |
| 13 | 4.96 | 98.7 | 9.97 |
| 14 | 4.5 | 103.29 | 5.47 |
| 15 | 4.44 | 103.86 | 1.94 |

The HSQC NMR spectrum of a batch of the oligosaccharide composition before and after spray drying are substantially similar to one another, with only minor variations in the relative size of each of the peaks. Thus, these HSQC NMR data demonstrate that spray drying has little-to-no effect on the HSQC NMR spectrum and chemical properties of the selected oligosaccharide composition.

### Example 18. HSQC NMR Analysis of Large-Scale Batches of the Selected Oligosaccharide Composition Using a Bruker NMR Machine

A determination of HSQC NMR spectra of samples produced using a large-scale process similar to the process as described in Example 2 (citric acid catalyst), was obtained using a Bruker NMR machine and analyzed using the MNova software package from Mestrelab Research, according to the HSQC NMR methods described in Example 9.

### Results

Two batches of the selected oligosaccharide composition produced according to the process described in Example 13 (500 kg galactose; 2000 L scale) (having an average DP of 14.0) were analyzed using the HSQC NMR methods described in Example 9. Prior to HSQC NMR analysis, samples were de-monomerized (e.g., using an ethanol precipitation method).

The relative size of each of the peaks collected for the NMR spectra of the analyzed batches of the selected oligosaccharide composition was determined, as shown below:

| | Center Position | | Large-scale batch #1 | Large-scale batch #2 |
|---|---|---|---|---|
| | (ppm) | | (% of total areas of | (% of total areas of |
| Signal | ¹H | ¹³C | signals 1-15) | signals 1-15) |
| 1 | 3.68 | 63.42 | 24.56 | 22.53 |
| 2 | 3.75 | 66.06 | 4.97 | 4.94 |
| 3 | 3.97 | 66.15 | 3.09 | 3.17 |
| 4 | 3.96 | 69.28 | 2.73 | 2.45 |
| 5 | 3.96 | 70.62 | 11.23 | 10.83 |
| 6 | 3.92 | 71.26 | 2.42 | 2.27 |
| 7 | 3.55 | 71.34 | 4.85 | 5.57 |
| 8 | 3.97 | 71.56 | 3.75 | 4.01 |
| 9 | 3.72 | 72.35 | 7.7 | 8.6 |
| 10 | 3.33 | 73.74 | 10.71 | 11.27 |
| 11 | 4.06 | 77.34 | 3.69 | 3.57 |
| 12 | 4.11 | 81.59 | 3.06 | 3.05 |
| 13 | 4.96 | 98.7 | 11.26 | 11.55 |
| 14 | 4.5 | 103.29 | 4.24 | 4.49 |
| 15 | 4.44 | 103.86 | 1.74 | 1.69 |

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising.” “having.” “including.” and “containing” are to be construed as open-ended terms (i.e., meaning “including, but not limited to.”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A method of treating and/or preventing an inflammatory lung disease or disorder in a subject, the method comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I), Formula (II), and Formula (III): 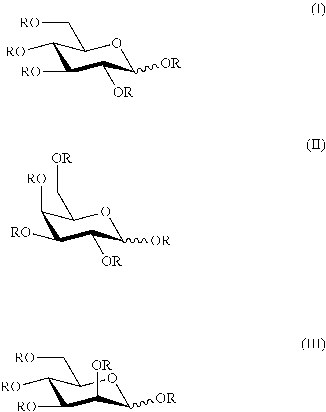
wherein each R independently is selected from hydrogen, and Formulae (Ia), (Ib), (Ic), (Id), (IIa), (IIb), (IIc), (IId), (IIa), (IIIb), (IIIc), (IIId): 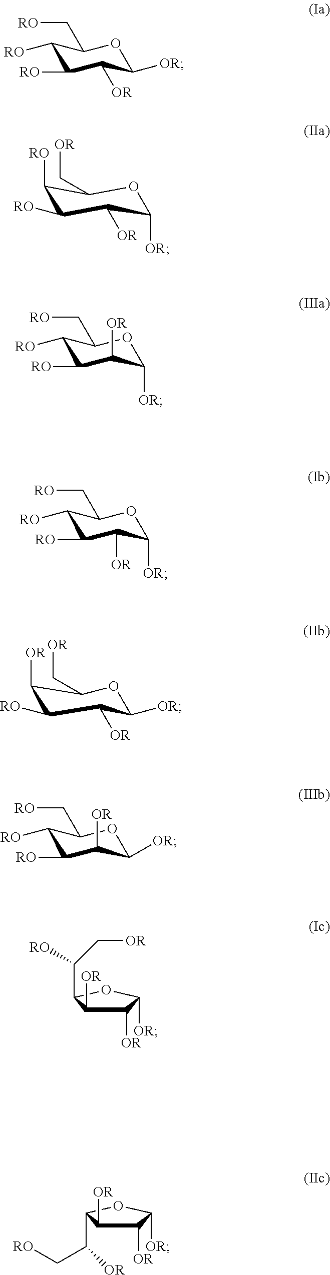 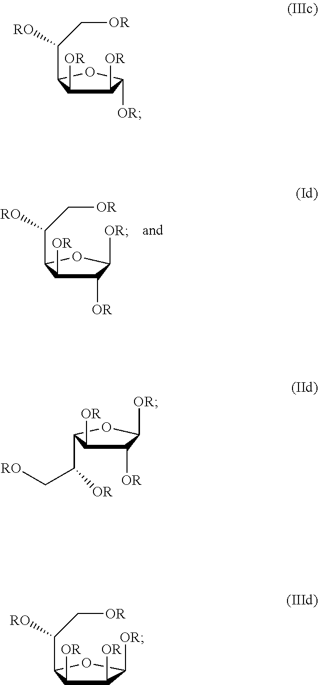
wherein each R independently is as defined above;
thereby treating and/or preventing the inflammatory lung disease or disorder.

2. A method comprising:
(A) identifying a subject having an inflammatory lung disease or disorder; and
(B) treating the subject with an oligosaccharide composition having an average degree of polymerization of 5-20 and comprises a plurality of oligosaccharides selected from Formula (I), Formula (II), and Formula (III): 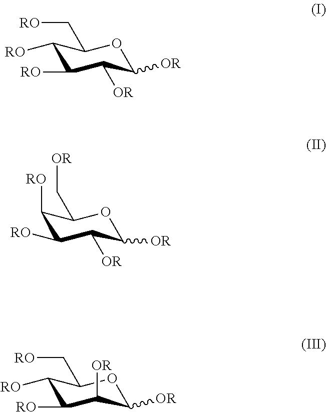
wherein each R independently is selected from hydrogen, and Formulae (Ia), (Ib), (Ic), (Id), (IIa), (IIb), (IIc), (IId), (IIIa), (IIIb), (IIIc), (IIId): 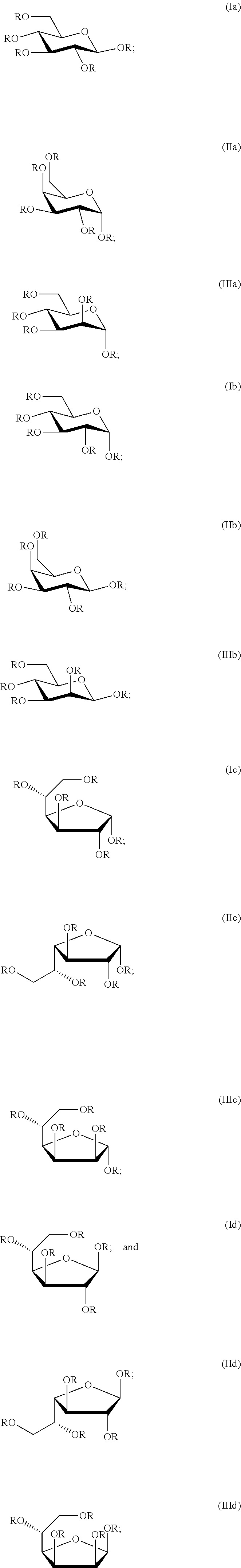
wherein each R independently is as defined above.

3. A method of treating and/or preventing having an inflammatory lung disease or disorder in a subject, the method comprising administering to the gastrointestinal tract of the subject an effective amount of an oligosaccharide composition, wherein the oligosaccharide composition is produced from a mixture comprising glucose, galactose, and mannose,
thereby treating and/or preventing inflammatory lung disease or disorder.

4. (canceled)

5. (canceled)

6. The method of any one of claims 1-3, wherein the subject has dysbiosis of the lung microbiome

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The method of claim 1, wherein the subject has increased relative or absolute abundance of pathogenic bacteria in their lungs relative to a healthy subject.

27. The method of claim 26, wherein administration of the oligosaccharide composition reduces the relative or absolute abundance of pathogenic bacteria in the lungs, sputum and/or gut of the subject relative to a control subject or a baseline measurement.

28. The method of claim 26, wherein the subject has increased levels of Moraxella catarrhalis, Haemophilus influenzae and/or Streptococcus pneumoniae in their lungs relative to a healthy subject.

29. (canceled)

30. The method of any one of claims 1-3, wherein the oligosaccharide composition promotes increased relative or absolute abundance of commensal bacteria in the lungs, sputum and/or gut of the subject.

31. The method of claim 30, wherein the oligosaccharide composition promotes increased relative or absolute abundance of one or more of Lactobacillus, Parabacteroides and Bacteroides.

32. The method of any one of claims 1-3, wherein the subject is more susceptible to respiratory viral infections than a healthy subject.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. The method of any one of claims 1-3, wherein the method prevents the inflammatory lung disease or disorder.

78. The method of claim 77, wherein the inflammatory lung disease or disorder is a viral respiratory infection.
